SEQUENCE LISTING (44 sequences)

SEQ ID NO: 1 - DNA, 1320 bp, Psilocybe cubensis
  source 1..1320: mol_type = genomic DNA
atgcaggtgatacccgcgtgcaactcggcagcaataagatcactatgtcctactcccgagtcttttagaaacatgggatggctctctgtcagcgatgcggtctacagcgagttcataggagagttggctacccgcgcttccaatcgaaattactccaacgagttcggcctcatgcaacctatccaggaattcaaggctttcattgaaagcgacccggtggtgcaccaagaatttattgacatgttcgagggcattcaggactctccaaggaattatcaggaactatgtaatatgttcaacgatatctttcgcaaagctcccgtctacggagaccttggccctcccgtttatatgattatggccaaattaatgaacacccgagcgggcttctctgcattcacgagacaaaggttgaaccttcacttcaaaaaacttttcgatacctggggattgttcctgtcttcgaaagattctcgaaatgttcttgtggccgaccagttcgacgacagacattgcggctggttgaacgagcgggccttgtctgctatggttaaacattacaatggacgcgcatttgatgaagtcttcctctgcgataaaaatgccccatactacggcttcaactcttacgacgacttctttaatcgcagatttcgaaaccgagatatcgaccgacctgtagtcggtggagttaacaacaccaccctcatttctgctgcttgcgaatcactttcctacaacgtctcttatgacgtccagtctctcgacactttagttttcaaaggagagacttattcgcttaagcatttgctgaataatgaccctttcaccccacaattcgagcatgggagtattctacaaggattcttgaacgtcaccgcttaccaccgatggcacgcacccgtcaatgggacaatcgtcaaaatcatcaacgttccaggtacctactttgcgcaagccccgagcacgattggcgaccctatcccggataacgattacgacccacctccttaccttaagtctcttgtctacttctctaatattgccgcaaggcaaattatgtttattgaagccgacaacaaggaaattggcctcattttccttgtgttcatcggcatgaccgaaatctcgacatgtgaagccacggtgtccgaaggtcaacacgtcaatcgtggcgatgacttgggaatgttccatttcggtggttcttcgttcgcgcttggtctgaggaaggattgcagggcagagatcgttgaaaagttcaccgaacccggaacagtgatcagaatcaacgaagtcgtcgctgctctaaaggcttag

SEQ ID NO: 2 - protein, 439 aa, Psilocybe cubensis
MQVIPACNSAAIRSLCPTPESFRNMGWLSVSDAVYSEFIGELATRASNRNYSNEFGLMQPIQEFKAFIESDPVVHQEFIDMFEGIQDSPRNYQELCNMFNDIFRKAPVYGDLGPPVYMIMAKLMNTRAGFSAFTRQRLNLHFKKLFDTWGLFLSSKDSRNVLVADQFDDRHCGWLNERALSAMVKHYNGRAFDEVFLCDKNAPYYGFNSYDDFFNRRFRNRDIDRPVVGGVNNTTLISAACESLSYNVSYDVQSLDTLVFKGETYSLKHLLNNDPFTPQFEHGSILQGFLNVTAYHRWHAPVNGTIVKIINVPGTYFAQAPSTIGDPIPDNDYDPPPYLKSLVYFSNIAARQIMFIEADNKEIGLIFLVFIGMTEISTCEATVSEGQHVNRGDDLGMFHFGGSSFALGLRKDCRAEIVEKFTEPGTVIRINEVVAALKA

SEQ ID NO: 3 - DNA, 2155 bp, Psilocybe cubensis
  source 1..2155: mol_type = genomic DNA
atgatcgctgtactattctccttcgtcattgcaggatgcatatactacatcgtttctcgtagagtgaggcggtcgcgcttgccaccagggccgcctggcattcctattcccttcattgggaacatgtttgatatgcctgaagaatctccatggttaacatttctacaatggggacgggattacagtctgtcttgccgcgttgacttctaatatatgaacagctaatatattgtcagacaccgatattctctacgtggatgctggagggacagaaatggttattcttaacacgttggagaccattaccgatctattagaaaagcgagggtccatttattctggccggtgagctgatgttgagttttttgcaattgaatttgtggtcacacgtttccagacttgagagtacaatggtcaacgaacttatggggtgggagtttgacttagggttcatcacatacggcgacaggtggcgcgaagaaaggcgcatgttcgccaaggagttcagtgagaagggcatcaagcaatttcgccatgctcaagtgaaagctgcccatcagcttgtccaacagcttaccaaaacgccagaccgctgggcacaacatattcgccagtaagtactacttgaggaaaatagcgtacgcttcgctgaccggtccgtacatcaaagtcagatagcggcaatgtcactggatattggttatggaattgatcttgcagaagacgacccttggctggaagcgacccatttggctaatgaaggcctcgccatagcatcagtgccgggcaaattttgggtcgattcgttcccttctcgtgagcatccttcttctatgtaggaagggaaggagtctaacaagtgttagtaaaataccttcctgcttggttcccaggtgctgtcttcaagcgcaaagcgaaggtctggcgagaagccgccgaccatatggttgacatgccttatgaaactatgaggaaattagcagttagtcaaatgcgttctccccgtattttttcaatactctaacttcagctcacagcctcaaggattgactcgtccgtcgtatgcttcagctcgtctgcaagccatggatctcaacggtgaccttgagcatcaagaacacgtaatcaagaacacagccgcagaggttaatgtcggtaagtcaaaagcgtccgtcggcaattcaaaattcaggcgctaaagtgggtcttctcaccaaggtggaggcgatactgtaaggatttctcaatcgttagagtataagtgttctaatgcagtacatactccaccaaccagactgtctctgctatgtctgcgttcatcttggccatggtgaagtaccctgaggtccagcgaaaggttcaagcggagcttgatgctctgaccaataacggccaaattcctgactatgacgaagaagatgactccttgccatacctcaccgcatgtatcaaggagcttttccggtggaatcaaatcgcacccctcgctataccgcacaaattaatgaaggacgacgtgtaccgcgggtatctgattcccaagaacactctagtcttcgcaaacacctggtgaggctgtccattcattcctagtacatccgttgccccactaatagcatcttgataacagggcagtattaaacgatccagaagtctatccagatccctctgtgttccgcccagaaagatatcttggtcctgacgggaagcctgataacactgtacgcgacccacgtaaagcggcatttggctatggacgacgaaattggtaagtgcgctttcagaacccccccttccgttgactagtgccatgcgcgcatacaatatcgctattgatctgatataacttccctgcggcatttattttggcattcctttagtcccggaattcatctagcgcagtcgacggtttggattgcaggggcaaccctcttatcagcgttcaatatcgagcgacctgtcgatcagaatgggaagcccattgacataccggctgattttactacaggattcttcaggtagctaatttccgtctttgtgtgcataatacccctaacgacgcacgtttacctttttgtaaagacacccagtgcctttccagtgcaggtttgttcctcgaacagagcaagtctcacagtcggtatccggaccctga

SEQ ID NO: 4 - protein, 508 aa, Psilocybe cubensis
MIAVLFSFVIAGCIYYIVSRRVRRSRLPPGPPGIPIPFIGNMFDMPEESPWLTFLQWGRDYNTDILYVDAGGTEMVILNTLETITDLLEKRGSIYSGRLESTMVNELMGWEFDLGFITYGDRWREERRMFAKEFSEKGIKQFRHAQVKAAHQLVQQLTKTPDRWAQHIRHQIAAMSLDIGYGIDLAEDDPWLEATHLANEGLAIASVPGKFWVDSFPSLKYLPAWFPGAVFKRKAKVWREAADHMVDMPYETMRKLAPQGLTRPSYASARLQAMDLNGDLEHQEHVIKNTAAEVNVGGGDTTVSAMSAFILAMVKYPEVQRKVQAELDALTNNGQIPDYDEEDDSLPYLTACIKELFRWNQIAPLAIPHKLMKDDVYRGYLIPKNTLVFANTWAVLNDPEVYPDPSVFRPERYLGPDGKPDNTVRDPRKAAFGYGRRNCPGIHLAQSTVWIAGATLLSAFNIERPVDQNGKPIDIPADFTTGFFRHPVPFQCRFVPRTEQVSQSVSGP

SEQ ID NO: 5 - DNA, 1089 bp, Psilocybe cubensis
  source 1..1089: mol_type = genomic DNA
atggcgttcgatctcaagactgaagacggcctcatcacatatctcactaaacatctttctttggacgtcgacacgagcggagtgaagcgccttagcggaggctttgtcaatgtaacctggcgcattaagctcaatgctccttatcaaggtcatacgagcatcatcctgaagcatgctcagccgcacatgtctacggatgaggattttaagataggtgtagaacgttcggtttacgaataccaggctatcaagctcatgatggccaatcgggaggttctgggaggcgtggatggcatagtttctgtgccagaaggcctgaactacgacttagagaataatgcattgatcatgcaagatgtcgggaagatgaagacccttttagattatgtcaccgccaaaccgccacttgcgacggatatagcccgccttgttgggacagaaattggggggttcgttgccagactccataacataggccgcgagaggcgagacgatcctgagttcaaattcttctctggaaatattgtcggaaggacgacttcagaccagctgtatcaaaccatcatacccaacgcagcgaaatatggcgtcgatgaccccttgctgcctactgtggttaaggaccttgtggacgatgtcatgcacagcgaagagacccttgtcatggcggacctgtggagtggaaatattcttctccagttggaggagggaaacccatcgaagctgcagaagatatatatcctggattgggaactttgcaagtacggcccagcgtcgttggacctgggctatttcttgggtgactgctatttgatatcccgctttcaagacgagcaggtcggtacgacgatgcggcaagcctacttgcaaagctatgcgcgtacgagcaagcattcgatcaactacgccaaagtcactgcaggtattgctgctcatattgtgatgtggaccgactttatgcagtgggggagcgaggaagaaaggataaattttgtgaaaaagggggtagctgcctttcacgacgccaggggcaacaacgacaatggggaaattacgtctaccttactgaaggaatcatccactgcgtaa

SEQ ID NO: 6 - protein, 309 aa, Psilocybe cubensis
MHIRNPYRTPIDYQALSEAFPPLKPFVSVNADGTSSVDLTIPEAQRAFTAALLHRDFGLTMTIPEDRLCPTVPNRLNYVLWIEDIFNYTNKTLGLSDDRPIKGVDIGTGASAIYPMLACARFKAWSMVGTEVERKCIDTARLNVVANNLQDRLSILETSIDGPILVPIFEATEEYEYEFTMCNPPFYDGAADMQTSDAAKGFGFGVGAPHSGTVIEMSTEGGESAFVAQMVRESLKLRTRCRWYTSNLGKLKSLKEIVGLLKELEISNYAINEYVQGSTRRYAVAWSFTDIQLPEELSRPSNPELSSLF

SEQ ID NO: 7 - DNA, 930 bp, Psilocybe cubensis
  source 1..930: mol_type = genomic DNA
atgcatatcagaaatccttaccgtacaccaattgactatcaagcactttcagaggccttccctcccctcaagccatttgtgtctgtcaatgcagatggtaccagttctgttgacctcactatcccagaagcccagagggcgttcacggccgctcttcttcatcgtgacttcgggctcaccatgaccataccagaagaccgtctgtgcccaacagtccccaataggttgaactacgttctgtggattgaagatattttcaactacacgaacaaaaccctcggcctgtcggatgaccgtcctattaaaggcgttgatattggtacaggagcctccgcaatttatcctatgcttgcctgtgctcggttcaaggcatggtctatggttggaacagaggtcgagaggaagtgcattgacacggcccgcctcaatgtcgtcgcgaacaatctccaagaccgtctctcgatattagagacatccattgatggtcctattctcgtccccattttcgaggcgactgaagaatacgaatacgagtttactatgtgtaaccctccattctacgacggtgctgccgatatgcagacttcggatgctgccaaaggatttggatttggcgtgggcgctccccattctggaacagtcatcgaaatgtcgactgagggaggtgaatcggctttcgtcgctcagatggtccgtgagagcttgaagcttcgaacacgatgcagatggtacacgagtaacttgggaaagctgaaatccttgaaagaaatagtggggctgctgaaagaacttgagataagcaactatgccattaacgaatacgttcaggggtccacacgtcgttatgccgttgcgtggtctttcactgatattcaactgcctgaggagctttctcgtccctctaaccccgagctcagctctcttttctag

SEQ ID NO: 8 - protein, 309 aa, Psilocybe cubensis; identical to SEQ ID NO: 6

SEQ ID NO: 9 - DNA, 3042 bp, Psilocybe cubensis
  source 1..3042: mol_type = genomic DNA
atgccttccagtcaccctcacattactcatcgctatcgggttccttcgagtgacgaccatgaacgtatatctgctctgttcttgggtcccaaagcagaaaatgccgcatttctccagcaatggttgaccacggtcgtcgcacagcaaaaggctgcccgcgatgcatacttcccggatgacaatgcttttattactacagacatgcaaacttcccccgcctttgctcagactactaaagtaatcgcctccaatctcaccgaattattgactgcactcggtgaaaggtcgattcctttcttctcacctcggtacagcggccatatgtctgtggaccaaagtctacctgccattctcggattcttatcgaccacattttataatcctaacaatgttgccttcgaggctagtccattcacgaccctcatcgaggaagaagttggcttgcaactctctgaaatgctgggttataatcggctaaataacaccgagaaacctctcgcctggggacatattgcatcaggtggaactgttgcaaacttggaagcgatgtgggcggcgcgaaacctcaagttttaccctctctcactccgtgatgcttcagccgaaggcgcagagatggaattcattcgtgacacattctccgtcaaaacctgtgttggtgacaaaaaattattaaaggattgcagcccatgggaactcctcaatttgcatgtttctactatcttagacatgcccgaccgtctgcacgacgagtacaatatttcacctcagttcctcgaaaaggttatgcgaaagtatatcatccagtctaccaacaaagacacgttgatgcagcgttggggacttacccaacaacctgtcgttttatccccgagcacaaaccattattcctggccaaaggctgcagctgtgctcggtattggctcagacaaccttcgcaacgtcccagtagacatccaagcccacatggacataaacgaactcgatcgtatgttaaaaatttgcttggacgaggagacgccagtatatcaagtagttgctgttatcggtaccaccgaagagggcggtgtcgatcgcattacggagatcctgaagctgcgccaaaagtatgaagctttggggctgtcttttgccatccatgcagatgctgcttggggaggctattttgcaaccatgctacccaaagatacattgggtcgaaaccggactaggcttcccaaagaggacactacctcgggctttgtccctcacgtcggtctgcgcgaggagagcgcgttacaactcagccatataaagtatgccgattctattactatcgacccgcacaaggcaggctatgttccttaccccgctggggcactctgttatcgcgacggaagaatgaggtacctgcttacatggtccgcgccctaccttgcccaaggcaacgagggccaaagtatcggaatatacgggatcgaaggaagcaaacctggtgcagcagcatccgcggtattcatggcgcacgaaaccattggcctgactccttctggatacgggaaccttcttggccaggcaatgtttacatgtcgccgatacgctgctcactggtctgcaatgtcaacggatactaccagtttcactgtcaccccgttcaatcctatccctgctgacatcgaccccaacgctgaccccgcaaaggtcgaagagcaaaaacagttcatcagagatcgtatcttgttcaaatcgaacgaggaaatatacaacgattctgaggctatggaactcttgcaccaacttgggtccgatctcaatatcaacgttttcgcatgcaacttccgcgaccgcgataataatctcaacaccgacgtcgaggaagccaactggctcaataaccgtattttccaacgcttttctgttacaagtgctgaggagaacccattggaaacgccattcttcctcagctcaactacattgaaacaatccgaatacggcgtctgcgcaaccgaagtaaagagacgcatgggacttgttggtgaccaggatgttatagtcctgaggaacgtcgttatgtctccatttactacaacgaacgactttgtgggaactctggcaaacaccttccaaaagatcgttgaggaggaggtcgagtatgcacggatccgcaacgatatgaaacctagcattcacaccttccttcttcatggttcaggagagcaatactatcttgtccacaccccaacgatccatatggccagcggccgtcgccaaatcatcctttcagtaaatgttgaaggccaagttcggcaggcgatacatgcccatgaaagagttgaagcagtgattgtacataacactgtgcccctccgccttgacgaaatcgttgacggaggatcatttgacggcatactcaccatcggaaagaggaaaactagtttcaaagtgaagatttcaaacattaaagtagtcaagaagcgctctctgatgactgaggacctggaatctgcgtacccatcgttgatgccattctatttctacgggactcaaggacacgctcatctcgaccatgtcattactgtcgttcctaacatccatctgagtgctggcgaaatacagtacaaattcgacgacgaggtgtcaagcgaggacctcgccaagggcctcattgttgttgctgagaacgtacacgaggcatccatgcagcccttcccgctcatgaaagatttcaagatcaccaaccaattcttcttcagctccgggcaaatactccgcgtcaaagtgtacagagatccatacccggcatcgacaatggatcccatccctctccacgacatcaagaaccagcccgtcgtgacacaaggcaccatcacgctcgtcggaaatatttacgtcgattctgatgcgctcaacgtcgcttccgagcctactgccgacgaagacgcggcgcatgttcctcacgctcgcaacatgtacggcgagatgaccgctggaacgatcaaaggctggcaaaacgctgttcgtcatttccacaacaaattggagactgttgctccgacgaagtag

SEQ ID NO: 10 - protein, 1013 aa, Psilocybe cubensis
MPSSHPHITHRYRVPSSDDHERISALFLGPKAENAAFLQQWLTTVVAQQKAARDAYFPDDNAFITTDMQTSPAFAQTTKVIASNLTELLTALGERSIPFFSPRYSGHMSVDQSLPAILGFLSTTFYNPNNVAFEASPFTTLIEEEVGLQLSEMLGYNRLNNTEKPLAWGHIASGGTVANLEAMWAARNLKFYPLSLRDASAEGAEMEFIRDTFSVKTCVGDKKLLKDCSPWELLNLHVSTILDMPDRLHDEYNISPQFLEKVMRKYIIQSTNKDTLMQRWGLTQQPVVLSPSTNHYSWPKAAAVLGIGSDNLRNVPVDIQAHMDINELDRMLKICLDEETPVYQVVAVIGTTEEGGVDRITEILKLRQKYEALGLSFAIHADAAWGGYFATMLPKDTLGRNRTRLPKEDTTSGFVPHVGLREESALQLSHIKYADSITIDPHKAGYVPYPAGALCYRDGRMRYLLTWSAPYLAQGNEGQSIGIYGIEGSKPGAAASAVFMAHETIGLTPSGYGNLLGQAMFTCRRYAAHWSAMSTDTTSFTVTPFNPIPADIDPNADPAKVEEQKQFIRDRILFKSNEEIYNDSEAMELLHQLGSDLNINVFACNFRDRDNNLNTDVEEANWLNNRIFQRFSVTSAEENPLETPFFLSSTTLKQSEYGVCATEVKRRMGLVGDQDVIVLRNVVMSPFTTTNDFVGTLANTFQKIVEEEVEYARIRNDMKPSIHTFLLHGSGEQYYLVHTPTIHMASGRRQIILSVNVEGQVRQAIHAHERVEAVIVHNTVPLRLDEIVDGGSFDGILTIGKRKTSFKVKISNIKVVKKRSLMTEDLESAYPSLMPFYFYGTQGHAHLDHVITVVPNIHLSAGEIQYKFDDEVSSEDLAKGLIVVAENVHEASMQPFPLMKDFKITNQFFFSSGQILRVKVYRDPYPASTMDPIPLHDIKNQPVVTQGTITLVGNIYVDSDALNVASEPTADEDAAHVPHARNMYGEMTAGTIKGWQNAVRHFHNKLETVAPTK

SEQ ID NO: 11 - DNA, 2103 bp, Psilocybe cubensis
  source 1..2103: mol_type = genomic DNA
atggaggctatcaaaaaggtttttgagaacaaaaaggcggagggcattcctgtgttggtgacctttgttactgcaggatatcctcgtcccgaagatactgttcccatcttgctggccatggaggccggtggtgctgatatcatcgagcttggtatgccattttcagacccaattgcagatggtcctgtcatccaggaaacgaacacaatcgccgttgcaaaccaggtagattataccactgttctcggacaacttcgggaagcccgcaaacaagggctcaaggcacccgttcttctgatgggatattataaccccatattggcttacggagaagacagatctattcaagatgcggctgaagctggagccaatgggtttattatggtcgaccttccacccgaggaggctgtcgcttttcgagagaaatgtatcaaatccaacctctcatatgttcctctaattgcaccctcaacgactctgtcgcgtataaagttcctctcaacaattgcagacacgttcatctatgtcgtgtctaaaatgggaaccaccggatcctcagagaaggttgccatgaataacgcccttcccaccatcatcgatcgtattcgcgagtacgctgaagttcctttagcagtcggatttggagtcgccactcgggctcacttcaactacgtcgccgattccggtgctgatggtgtcgttattggcaccaaactcgttaacgttattaaagagtcaccgcaaggggaagcacccaaaaatgttgaggcatactgccgtgagatgagccaaaagggagaaacaaatcgcgtcaaatctccaccaactgcccgtgctgccagctccgaatcaattcctgttgttgttccttctgttctccccgcacgtttcggagaattcggaggacaatacgttcccgaagctcttgtcgattgtctggttgaactagaagaagctcacaaatctgccatggctgatcctgaattccagaaggaactacaatcgcatgccggatatgcaaatcgtccttcacaaatatacctcgccgaaaatctcaccaaggatgctgggggtgcaaatatttggttgaaacgtgaagatttgaaccacacaggttcccacaaaatcaataacgctttgggacaaattctgcttgcccggagaatcggaaagaccagaattatcgcagaaacaggtgccggccagcatggtgttgcaacagcgactgtttgcgctaagtttggaatggaatgtgttatctacatgggcgcagaagatgtgcgacggcaagctctaaatgtattcaggattgagatgctaggagcaaaagttgttcctgttacttcaggatcatgcacattgaaggacgctgtaaacgaggccttccgtgactgggtgacaaacctttctacgacgcattatttggttggctctgtaattggacctcatcccttccccaccattgtccgagatttccaaaaggtcattggtcaagagatcaaggctcagatgttggccgcccgcggcaaacttcctgatgtcgtcgtcgcttgtgttggtggaggaagcaatgctatcggtacgttctatgattttattggcgacaagagtgtacgtctagttggggtggaagcaggaggagaaggtattgacggagaccgacatagcgccacactttcgatggggcaaccgggagtacttcacggtgttagaacatatattctacaagacaaggccggtcaaatcatcgagacgcactcaatcagcgctggattggattatcccggcgttggaccagaacatgcttggctaaaggactctaaaagagcagaatatgttgtcgccacagacgaagaagcacttcgcggtttccgtatgctaacacaaagggagggaattattcctgcccttgaatcttcccatgcgatctgggaggctgtcaggattgcccgcaccatgtcgaaggaccaggatcttgttgtgtgtttgtctggccgaggtgataaagacgttgagcaaatttctcaacttcttcccaagtgggcggatattctagactggcatgtttcttcccatgccgttggacacacaacaaaattctaa

SEQ ID NO: 12 - protein, 700 aa, Psilocybe cubensis [sequence omitted]

SEQ ID NO: 13 - DNA, 1572 bp, Oryza sativa
  source 1..1572: mol_type = genomic DNA
atggagctcaccatggcgtcgacgatgtcgctcgcgctgctcgtgctctccgcggcgtacgtgttggtcgcgttgaggaggagccggtcgtcgtcgtcaaagccacggcggctgccgccgtcgccgccggggtggccggtgatcgggcacctccacctcatgtccggcatgccgcaccacgcgctggccgagctggcgcgcaccatgcgcgcgccgctgttccggatgcggctggggagcgtgccggcggtggtgatctccaagccggacctcgcccgcgccgcgctcaccaccaacgacgccgcgctggcgtcgcggccgcacctgctctccggccagttcctgtcgttcggctgctccgacgtgacgttcgcgccggcggggccgtaccaccggatggcgcgccgcgtggtggtgtcggagctcctgtcggcgcgtcgcgtcgccacgtacggcgccgtcagggtcaaggagctccgccgcctgctcgcgcacctcaccaagaacacctcgccggcgaagcccgtcgacctcagcgagtgcttcctcaacctcgccaacgacgtgctctgccgcgtcgcgttcggccgccggttcccgcacggcgagggcgacaagctcggcgcggtgctcgccgaggcgcaggacctcttcgccgggttcaccatcggcgacttcttccccgagctcgagcccgtcgccagcaccgtcaccggactccgccgccgcctcaagaagtgcctcgccgacctccgcgaggcctgcgacgtgatcgtggacgaacacatcagcggcaaccgccagcgcatccccggcgaccgcgacgaggacttcgtcgacgtcctcctccgcgtccagaaatcccccgacctcgaggtccccctaaccgacgacaatctcaaggccctcgtcctggacatgttcgtcgccggcacggacaccacgttcgcgacgctggagtgggtgatgacggagctagtccgccacccacggatcctcaagaaggcgcaggaggaggtccggcgagtcgtcggcgacagcggccgcgtcgaggagtcccacctcggcgagctccactacatgcgcgccatcatcaaggagacgttccggctgcacccggcggtgccgttgctagtgccgcgcgagtccgtcgcgccgtgcacgctgggcggctacgacatcccggcgaggacgcgggtgttcatcaacacgttcgccatggggcgcgacccggagatctgggacaacccgctggagtactcgccggagaggttcgagagcgccggcggcggcggcgagatcgacctcaaggacccggactacaagctgctgccgttcggcggcgggcggcgagggtgccccggctacacgttcgcgctcgccaccgtgcaggtgtcgctcgccagcttgctctaccacttcgagtgggcgctgcccgccggcgtgcgcgccgaggacgtcaacctcgacgagacgttcggcctcgccacgaggaagaaggagccgctcttcgtcgccgtcaggaagagcgacgcgtacgagtttaagggagaggagcttagtgaggtttaa

SEQ ID NO: 14 - protein, 522 aa, Oryza sativa
MELTMASTMSLALLVLSAAYVLVALRRSRSSSSKPRRLPPSPPGWPVIGHLHLMSGMPHHALAELARTMRAPLFRMRLGSVPAVVISKPDLARAALTTNDAALASRPHLLSGQFLSFGCSDVTFAPAGPYHRMARRVVVSELLSARRVATYGAVRVKELRRLLAHLTKNTSPAKPVDLSECFLNLANDVLCRVAFGRRFPHGEGDKLGAVLAEAQDLFAGFTIGDFFPELEPVASTVTGLRRRLKKCLADLREACDVIVDEHISGNRQRIPGDRDEDFVDVLLRVQKSPDLEVPLTDDNLKALVLDMFVAGTDTTFATLEWVMTELVRHPRILKKAQEEVRRVVGDSGRVEESHLGELYMRAIIKETFRLHPAVPLLVPRESVAPCTLGGYDIPARTRVFINTFAMGRDPEIWDNPLEYSPERFESAGGGGEIDLKDPDYKLLPFGGGRRGCPGYTFALATVQVSLASLLYHFEWALPAGVRAEDVNLDETFGLATRKKEPLFVAVRKSDAYEFKGEELSEV

SEQ ID NO: 15 - DNA, 1335 bp, Rattus norvegicus
  source 1..1335: mol_type = genomic DNA
atgattgaagacaacaaggagaacaaagaccattcctcagaaagggggagagtgactctcatcttttccttgaagaatgaagttggaggactcataaaagcactgaaaatcttccaggagaaccacgtgaacctgttacatattgagtcccggaaatcgaagcgaagaaactcagaatttgagatttttgtggactgcgacatcaaccgagaacagctgaatgacatctttcccctgctgaagtcccacaccacggtcctctctgtggactcgcccgatcagctccctgaaaaggaagatgttatggagactgtcccttggttcccaaagaagatttctgacctggacttctgcgccaacagagtgctgttgtacggatccgaactcgacgcggaccatcctggcttcaaagacaatgtctaccgtagaagacgaaagtatttcgcagagctggctatgaactacaaacatggggaccccattcccaagattgaattcacggaagaggagattaagacctgggggaccatcttccgagagctgaacaaactctacccaacccacgcctgcagggagtacctcagaaaccttcctctgctctcaaaatactgtggctatcgggaagacaacgtcccgcaactggaagatgtctccaactttttaaaagaacgcacagggttttccatccgtcctgtggctggttacctctcaccgagagatttcctgtcagggttagcctttcgagtctttcactgcactcagtatgtgagacacagttcggatcctctctataccccagagccagacacctgccacgaactcttaggccacgtccctctcttggctgaacccagttttgctcaattctcccaagaaattggcctggcttctcttggagcttcagaggagacggttcagaaactggcaacgtgctacttcttcactgtggagtttggactgtgcaagcaagatgggcagctgagagtctttggtgccggcctgctttcttccatcagtgagctcagacatgcactttctggacatgccaaggttaagccctttgatcccaaggttgcctgcaaacaggaatgtctcatcacaagcttccaggatgtctactttgtatcggagagctttgaagatgcaaaggagaagatgagagaatttgccaaaaccgtgaagcgcccgtttggagtgaagtacaatccgtacacacagagcattcaggttctgagagacagcaagagcataaccagcgccatgaatgagttgcggcatgacctcgatgtcgtcaatgatgcccttgctagagtcagcaggtggcccagtgtgtga

SEQ ID NO: 16 - protein, 444 aa, Rattus norvegicus
MIEDNKENKDHSSERGRVTLIFSLKNEVGGLIKALKIFQENHVNLLHIESRKSKRRNSEFEIFVDCDINREQLNDIFPLLKSHTTVLSVDSPDQLPEKEDVMETVPWFPKKISDLDFCANRVLLYGSELDADHPGFKDNVYRRRRKYFAELAMNYKHGDPIPKIEFTEEEIKTWGTIFRELNKLYPTHACREYLRNLPLLSKYCGYREDNVPQLEDVSNFLKERTGFSIRPVAGYLSPRDFLSGLAFRVFHCTQYVRHSSDPLYTPEPDTCHELLGHVPLLAEPSFAQFSQEIGLASLGASEETVQKLATCYFFTVEFGLCKQDGQLRVFGAGLLSSISELRHALSGHAKVKPFDPKVACKQECLITSFQDVYFVSESFEDAKEKMREFAKTVKRPFGVKYNPYTQSIQVLRDSKSITSAMNELRHDLDVVNDALARVSRWPSV

SEQ ID NO: 17 - DNA, 1002 bp, Oryza sativa
  source 1..1002: mol_type = genomic DNA
atgcccgccgtggccgggagcttgtacatggcgagccagcacaagggagtgcctccgccgctgccgccgccgccgcggccattgccggtgatcaacctgggccggctcaccatggacagtgcctcgcgggcgctcgccgtgcgggatatcgtgctggcgtgccgtgaacgtggctgctttgaggtggtgaaccatggcatcagcaggtcttgcatgaatggcgccctcgaagccgcctccgagttcttccagctatcgacggagcgcaaggaggagttcgcgtcggacgacatccggcagcccatcaggtacgacacgagctcgagggacgggatcagcatgtccaggtcatttctgaagcactatgccaatcccctggacgactggatcaagttctggccgacgcagccaccaacttacagggagaaaatgggtgagtacgccgtggagacgcagcgagtgtcaatgcagctcatggaagcaatcctgcagggcctgggattagggccatcgtacctgcaagaaaagcttgaaggaggggtgcagttcgtggccttgaacaactacccgcagtcatcggcgaagaaagccgacaagatcggcttggctcctcactctgattatggcttcctcaccatcctgttgcagagctccccagggcttgaggtgatgcaccatgaggatgatgcctggacatctgtccctgctatccctggggctctccatgtccatgtaggagaccacctggaagtgttgagcaatggccagctcaagtcccttgtccatcgagccgttctcaacccaaacgagtcaaggatttctattgccagcatccatggtctctcgatggatgaagaggtccactgcgccgaagagctcgtcgatgaacaccaccccaaaatgtacaggggaagcagcttccaggacttcctggacttcctgccagcagacatgaacaggtataggaggtatgtcgagagcctcaggatcgacaaaccctga

SEQ ID NO: 18 - protein, 333 aa, Oryza sativa
MPAVAGSLYMASQHKGVPPPLPPPPRPLPVINLGRLTMDSASRALAVRDIVLACRERGCFEVVNHGISRSCMNGALEAASEFFQLSTERKEEFASDDIRQPIRYDTSSRDGISMSRSFLKHYANPLDDWIKFWPTQPPTYREKMGEYAVETQRVSMQLMEAILQGLGLGPSYLQEKLEGGVQFVALNNYPQSSAKKADKIGLAPHSDYGFLTILLQSSPGLEVMHHEDDAWTSVPAIPGALHVHVGDHLEVLSNGQLKSLVHRAVLNPNESRISIASIHGLSMDEEVHCAEELVDEHHPKMYRGSSFQDFLDFLPADMNRYRRYVESLRIDKP

SEQ ID NO: 19 - DNA, 1539 bp, Aspergillus nidulans
  source 1..1539: mol_type = genomic DNA
atgctgagtctcgacctagtattctctttcccagcatgggcgctcctcctagtcctaaccctcttgtatacactatacctagccacaacgcgcctcctcctgagccccattcgccatattcccggcccaactctagccgcactgtcgttttggcctgaattctactacgacgtcgtccagcggggacagtattttcgacaaattgacaagatgcatcagacatacggtccgctagtccgcattaacccattcgaaatccacatccaagacccctccttctatcccgtgctgtacacaggcccaactcgtcgacgacataaatggctctgggctgcacgaatgttcgggaacaacacctctgcctttgcaaccgtccggcacgaacaccacagactaagaaggagtgcattgaaccctctcttctcaaagagcgctatccagcggctaacaccgcacctgcaacataccctagcgcggctatgtagtcggctcgatgggttcgcttttacaagacaggatgtggacttggggatcggacttacagcgttcgcggccgacgtcatcacagagtactgtttcggacagtcactggagttgattgggaaagataactttggaaaggagtggattgatatggtaagcgctccgtcagagctcggccatctggtaaagcagtgtccgtggatcttggtggtttgtaaatgggcgccgaaagctcttgttagggctttgctgccgggggttgcgttgttgtttcaaatacaagagagaatgagcgcccagattcaaccgcttgttgatcgagccgccgccgttgacaagccagctgaccctttgaccgtcttcgacttcctcctctcgagtaccctcccacagcacgaaaagacagtggaccgactcaagggcgaagggcagacgctcatcggcgcggggacattaacgaccgggaatgcattgaagacgataatcttccacgtcctcaatgatcccgacatctttcggaaacttcgagcagaggtcgacggtgctctagaaaatatggatattctgtccatgtctgatacggcctacctcgaacgcctcccgtacctgtcagcgtgcataaaggaaggtctacggatttcctacggcgtcacgcatagactccaactgatcgccgaagagcccctaatatactcaggagtgaccatcccagcaggaacaccagttggaatgacatcgatcttcatgcacgacaatccagtagtattccctcagccgcgggaattccgtccagagcgctggttcgaggcagattttgagactgtgcaggcaatgaatcgccacttcgtccccttcagcaagggcagccgcatgtgtctgggaatgaacctggcgtatgcggagatttacttggtgctggcagtactttttcgacggtatgagatttctctgagcggggtgacgagggaggatattgagatggcacatgattttttcgatcctgcgccaaaggagggggcaagaggattgatcgttcaactgcagaagagggggtga

SEQ ID NO: 20 - protein, 512 aa, Aspergillus nidulans
MLSLDLVFSFPAWALLLVLTLLYTLYLATTRLLLSPIRHIPGPTLAALSFWPEFYYDVVQRGQYFRQIDKMHQTYGPLVRINPFEIHIQDPSFYPVLYTGPTRRRHKWLWAARMFGNNTSAFATVRHEHHRLRRSALNPLFSKSAIQRLTPHLQHTLARLCSRLDGFAFTRQDVDLGIGLTAFAADVITEYCFGQSLELIGKDNFGKEWIDMVSAPSELGHLVKQCPWILVVCKWAPKALVRALLPGVALLFQIQERMSAQIQPLVDRAAAVDKPADPLTVFDFLLSSTLPQHEKTVDRLKGEGQTLIGAGTLTTGNALKTIIFHVLNDPDIFRKLRAEVDGALENMDILSMSDTAYLERLPYLSACIKEGLRISYGVTHRLQLIAEEPLIYSGVTIPAGTPVGMTSIFMHDNPVVFPQPREFRPERWFEADFETVQAMNRHFVPFSKGSRMCLGMNLAYAEIYLVLAVLFRRYEISLSGVTREDIEMAHDFFDPAPKEGARGLIVQLQKRG

SEQ ID NO: 21 - DNA, 3810 bp, synthetic construct
  misc_feature 1..3810: note = pCDM4 vector
  source 1..3810: mol_type = other DNA
gctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagcctaggatcgagatcgatctcgatcccgcgaaattaatacgactcactataggggaattgtgagcggataacaattcccctctagaaataattttgtttaactttaagaaggagatatacatatggcagatctcaattggatatcggccggccacgcgatcgctgacgtcggtaccctcgagtctggtaaagaaaccgctgctgcgaaatttgaacgccagcacatggactcgtctactagtcgcagcttaattaacctaaactgctgccaccgctgagcaataactagcataaccccttggggcctctaaacgggtcttgaggggttttttgctagcgaaaggaggagtcgacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagccagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggccc

SEQ ID NO: 22 - DNA, 24 bp, synthetic construct
  misc_feature 1..24: note = Synthetic FLAG epitope tag polypeptide
  source 1..24: mol_type = other DNA
gactacaaggatgacgatgacaaa

SEQ ID NO: 23 - protein, 8 aa, synthetic construct
  REGION 1..8: note = Synthetic FLAG epitope tag polypeptide
DYKDDDDK

SEQ ID NO: 24 - DNA, 549 bp, Streptomyces griseofuscus
  source 1..549: mol_type = genomic DNA
atgaacaccttcagaacagccactgccagagacatacctgatgtagcagcaactcttacggaagccttcgcaactgatccacccacgcagtgggtgttccccgacggtactgccgccgtcagcaggttctttacacatgttgcagatagggttcacacggccggtggtattgttgagctactaccagacagagccgccatgattgcattgccaccacacgtgaggctgccaggagaagctgccgacggaaggcaggcggaaattcagagaaggctggcagacaggcacccgctgacacctcactactacctgctgttttacggagttagaacggcacaccagggttcgggattgggcggaagaatgctggccagattaactagcagagctgatagggacagggtgggtacatatactgaggcatccacctggcgtggcgctagactgatgctgagacatggattccatgctacaaggccactaagattgccagatggacccagcatgtttccactttggagagatccaatccatgatcattctgattag

SEQ ID NO: 25 - protein, 182 aa, Streptomyces griseofuscus
MNTFRTATARDIPDVAATLTEAFATDPPTQWVFPDGTAAVSRFFTHVADRVHTAGGIVELLPDRAAMIALPPHVRLPGEAADGRQAEIQRRLADRHPLTPHYYLLFYGVRTAHQGSGLGGRMLARLTSRADRDRVGTYTEASTWRGARLMLRHGFHATRPLRLPDGPSMFPLWRDPIHDHSD

SEQ ID NO: 26 - DNA, 1170 bp, Thermotoga maritima
  source 1..1170: mol_type = genomic DNA
atgaaaggatatttcggaccatacggtggccagtacgtaccagaaatattaatgggtgccttagaggagttagaggcagcatacgaggagattatgaaggatgagagcttctggaaggagttcaacgatctactgagggattacgcaggcagaccaacgccattgtactttgccaggagattgtctgagaagtacggcgcccgtgtttacttgaagcgtgaggatctgctgcacactggagcacacaagataaataacgctatcggacaggttttattggccaaattaatggggaagacacgtatcatagccgagacgggagctgggcagcatggagtcgctactgctaccgctgctgccctgttcggaatggaatgtgtgatctacatgggtgaagaggacacaatcagacagaagttgaacgtggagcgtatgaaattattaggggctaaagttgtccctgttaagtctggcagtaggaccttgaaggatgcgatagacgaggctttgagagactggattactaatttacagacaacatattatgttatcggatctgttgttggtccccacccttacccaattatcgtaaggaatttccagaaggttatcggtgaggagaccaagaagcaaataccagaaaaggaaggtcgtttgccagactatatagttgcctgcgtaggcggcggtagcaatgccgcaggtatattttacccattcatagactctggagtaaagctgataggtgttgaggcaggtggcgagggattggagacaggtaaacacgcagcctcgttattaaagggtaaaattggctatttacatggatcgaagacctttgttctacaagatgactggggtcaagtccaagtgagccattcggtgtcagctggtcttgactattcaggagtaggacctgagcatgcttattggagagagacagggaaggttctgtacgacgcagtgactgacgaagaggctttggacgcatttatagagttatcaagactagagggcattatacccgctttagagtcatcgcatgctctagcatatttgaagaagataaatataaaaggtaaggttgtggtggtcaacctatcagggagaggggataaagacctggagtcagtcttaaaccatccatacgtgagagaaagaattagatga

SEQ ID NO: 27 - protein, 389 aa, Thermotoga maritima
MKGYFGPYGGQYVPEILMGALEELEAAYEEIMKDESFWKEFNDLLRDYAGRPTPLYFARRLSEKYGARVYLKREDLLHTGAHKINNAIGQVLLAKLMGKTRIIAETGAGQHGVATATAAALFGMECVIYMGEEDTIRQKLNVERMKLLGAKVVPVKSGSRTLKDAIDEALRDWITNLQTTYYVIGSVVGPHPYPIIVRNFQKVIGEETKKQIPEKEGRLPDYIVACVGGGSNAAGIFYPFIDSGVKLIGVEAGGEGLETGKHAASLLKGKIGYLHGSKTFVLQDDWGQVQVSHSVSAGLDYSGVGPEHAYWRETGKVLYDAVTDEEALDAFIELSRLEGIIPALESSHALAYLKKINIKGKVVVVNLSGRGDKDLESVLNHPYVRERIR

SEQ ID NO: 28 - DNA, 42 bp, synthetic construct
  misc_feature 1..42: note = Synthetic V5 epitope tag polypeptide
  source 1..42: mol_type = other DNA
ggtaagccaattccaaatcctttgttgggtttggactccacc

SEQ ID NO: 29 - protein, 14 aa, synthetic construct
  REGION 1..14: note = Synthetic V5 epitope tag polypeptide
GKPIPNPLLGLDST

SEQ ID NO: 30 - DNA, 5203 bp, synthetic construct
  misc_feature 1..5203: note = pETM6-H10 vector
  source 1..5203: mol_type = other DNA
gaagaattgtgagcggataacaattcccctctagaaataattttgtttaactttaagaaggagatatacatatggcagatctcaattggatatcggccggccacgcgatcgctgacgtcggtaccctcgagtctggtaaagaaaccgctgctgcgaaatttgaacgccagcacatggactcgtctactagtcgcagcttaattaacctaaactgctgccaccgctgagcaataactagcataaccccttggggcctctaaacgggtcttgaggggttttttgctagcgaaaggaggagtcgactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagcctaggatcgagatcgatctcgatcccgcgaaattaatacgactcactacg

SEQ ID NO: 31 - DNA, 1446 bp, Bacillus atrophaeus
  source 1..1446: mol_type = genomic DNA
atgatgtctgaaaatttgcaattgtcagctgaagaaatgagacaattgggttaccaagcagttgatttgatcatcgatcacatgaaccatttgaagtctaagccagtttcagaaacaatcgattctgatatcttgagaaataagttgactgaatctatcccagaaaatggttcagatccaaaggaattgttgcatttcttgaacagaaacgtttttaatcaaattacacatgttgatcatccacatttcttggcttttgttccaggtccaaataattacgttggtgttgttgcagatttcttggcttctggttttaatgtttttccaactgcatggattgctggtgcaggtgctgaacaaatcgaattgactacaattaattggttgaaatctatgttgggttttccagattcagctgaaggtttatttgtttctggtggttcaatggcaaatttgacagctttgactgttgcaagacaggctaagttgaacaacgatatcgaaaatgctgttgtttacttctctgatcaaacacatttctcagttgatagagcattgaaggttttaggttttaaacatcatcaaatctgtagaatcgaaacagatgaacatttgagaatctctgtttcagctttgaagaaacaaattaaagaagatagaactaagggtaaaaagccattctgtgttattgcaaatgctggtactacaaattgtggtgctgttgattctttgaacgaattagcagatttgtgtaacgatgaagatgtttggttgcatgctgatggttcttatggtgctccagctatcttgtctgaaaagggttcagctatgttgcaaggtattcatagagcagattctttgactttagatccacataagtggttgttccaaccatacgatgttggttgtgttttgatcagaaactctcaatatttgtcaaagacttttagaatgatgccagaatacatcaaggattcagaaactaacgttgaaggtgaaattaatttcggtgaatgtggtatcgaattgtcaagaagattcagagctttgaaggtttggttgtcttttaaagttttcggtgttgctgcttttagacaagcaatcgatcatggtatcatgttagcagaacaagttgaagcatttttgggtaaagcaaaagattgggaagttgttacaccagctcaattgggtatcgttacttttagatacattccatctgaattggcatcaacagatactattaatgaaattaataagaaattggttaaggaaatcacacatagaggtttcgctatgttatctactacagaattgaaggaaaaggttgttattagattgtgttcaattaatccaagaactacaactgaagaaatgttgcaaatcatgatgaagattaaagcattggctgaagaagtttctatttcatacccatgtgttgctgaataa

SEQ ID NO: 32 - protein, 481 aa, Bacillus atrophaeus
MMSENLQLSAEEMRQLGYQAVDLIIDHMNHLKSKPVSETIDSDILRNKLTESIPENGSDPKELLHFLNRNVFNQITHVDHPHFLAFVPGPNNYVGVVADFLASGFNVFPTAWIAGAGAEQIELTTINWLKSMLGFPDSAEGLFVSGGSMANLTALTVARQAKLNNDIENAVVYFSDQTHFSVDRALKVLGFKHHQICRIETDEHLRISVSALKKQIKEDRTKGKKPFCVIANAGTTNCGAVDSLNELADLCNDEDVWLHADGSYGAPAILSEKGSAMLQGIHRADSLTLDPHKWLFQPYDVGCVLIRNSQYLSKTFRMMPEYIKDSETNVEGEINFGECGIELSRRFRALKVWLSFKVFGVAAFRQAIDHGIMLAEQVEAFLGKAKDWEVVTPAQLGIVTFRYIPSELASTDTINEINKKLVKEITHRGFAMLSTTELKEKVVIRLCSINPRTTTEEMLQIMMKIKALAEEVSISYPCVAE

SEQ ID NO: 33 - DNA, 18 bp, synthetic construct
  misc_feature 1..18: note = Synthetic HIS epitope tag polypeptide
  source 1..18: mol_type = other DNA
catcatcatcatcatcat

SEQ ID NO: 34 - protein, 6 aa, synthetic construct
  REGION 1..6: note = Synthetic HIS epitope tag polypeptide
HHHHHH

SEQ ID NO: 35 - DNA, 5369 bp, synthetic construct
  misc_feature 1..5369: note = pET28a(+) vector
  source 1..5369: mol_type = other DNA
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttgtcgacggagctcgaattcggatccgcgacccatttgctgtccaccagtcatgctagccatatggctgccgcgcggcaccaggccgctgctgtgatgatgatgatgatggctgctgcccatggtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 36 - DNA, 33 bp, synthetic construct
  misc_feature 1..33: note = Synthetic c-MYC epitope tag polypeptide
  source 1..33: mol_type = other DNA
gaacaaaagttaatttctgaagaagatttggaa

SEQ ID NO: 37 - protein, 10 aa, synthetic construct
  REGION 1..10: note = Synthetic c-MYC epitope tag polypeptide
EQKLISEEDL

SEQ ID NO: 38 - DNA, 2160 bp, Oryza sativa
  source 1..2160: mol_type = genomic DNA
atggagtcgtcggcggggccgatggagctggtggcggcgctgctgcgggggctgacgccgcgggcggagcagctgctgcagctgtcgtccgggggaggggaggccgccgccggtggcgcggcggaggcgagggccgcggtggccacggtggccgccgcgctgctcgggtgcgcgttcttggtgttatggaggcgggtctcggcggggcggaagcggaagagggaggaggcagagaggtcggcggcggccgtggctggggtggggaagggcgggaagaatgcctccgcggcggccggggaggaggccggcggcgccgacgggaggaagcgggtcaccgtcttcttcggcacgcagaccggcaccgccgagggcttcgccaaggcactcgctgaggaggctaagtcaagatacgacaaggcgatattcaaagttgtggacttggatgagtatgcgatggaggatgaggagtacgaggagagattgaagaaggagaagatatcgttgttcttcgttgcaacgtacggagatggtgaaccgactgacaatgctgctaggttctataaatggttcactgagggaaatgagaggggtgtttggttgaatgacttccagtatgctatttttggtcttggcaatcggcagtatgagcatttcaacaaggttgccaaggttgttgatgagctcctagttgagcaaggtggaaaacgtcttgttccggttggtcttggagatgatgatcaatgcattgaggatgacttcaacgcatggaaagaaactctctggccagaattggatcagttacttcgggatgaaaatgatgtttcaacaggcactacctacacagctgccattcctgaataccgggttgaatttgttaagcctgatgaggcagcccatttggagagaaatttcagtcttgcaaacggttatgcggttcatgatgctcagcatccttgccgggccaacgtggctgtgcgacgggaactccacactcctgcttctgatcgttcatgcactcacttggagtttgacattgctggcactggtcttacgtatgaaaccggtgaccatgttggtgtatacacagagaactgcctcgaggttgtagaggaggcagagaggttgttaggctactccccagaggcttttttcaccatccatgcagacaaagaggacggtacaccactaggtggtggttctctggctcctccattcccttccccgattactgtgaggaatgcgcttgctagatatgcggatcttctgaattcgccgaagaagagtgctttggttgcattagctacttatgcttcagattctactgaagctgatcgtctgaggttcttggcctctcctgctggaaaggatgagtatgctcaatgggttgttgcgagtcaaagaagtctattagaagtgatggcagagttcccttcagcaaagcctccactaggagtcttctttgcagccgttgctcctcgtcttcagccgagatactactcaatttcatcttcacctagcatggcacctaccagaattcatgttacatgtgcacttgtccatgaaaaaacacctgctggaagggtacataagggagtctgctcaacatggattaagaatgctattccatcagaagagacaaaggactgcagctgggctccagtttttgtgagacaatcaaacttcaaactgcctgctgatccttcagtaccggttatcatgattggcccaggaactggtcttgctcctttccgcggattcttgcaggagaggctgtctcaaaaacaatcaggagctgagcttggtcgctccgtattcttctttggatgcagaaacagcaagatggacttcatctatgaggatgagctgaacactttccttgaggaaggagcattgtccgagctggttctcgccttctctcgtgagggccctacgaaggaatacgtgcagcacaaaatgtcgcagaaagcttccgaaatctgggacatgatctcccagggtggttacatttacgtctgtggtgatgccaaaggcatggccagagatgtacatagagttctccacaccattgtacaggaacagggatcacttgacagctctaaggctgagagctttgtgaagagcctccaaacggagggtaggtatctgagagatgtgtggtga

SEQ ID NO: 39 - protein, 719 aa, Oryza sativa
MESSAGPMELVAALLRGLTPRAEQLLQLSSGGGEAAAGGAAEARAAVATVAAALLGCAFLVLWRRVSAGRKRKREEAERSAAAVAGVGKGGKNASAAAGEEAGGADGRKRVTVFFGTQTGTAEGFAKALAEEAKSRYDKAIFKVVDLDEYAMEDEEYEERLKKEKISLFFVATYGDGEPTDNAARFYKWFTEGNERGVWLNDFQYAIFGLGNRQYEHFNKVAKVVDELLVEQGGKRLVPVGLGDDDQCIEDDFNAWKETLWPELDQLLRDENDVSTGTTYTAAIPEYRVEFVKPDEAAHLERNFSLANGYAVHDAQHPCRANVAVRRELHTPASDRSCTHLEFDIAGTGLTYETGDHVGVYTENCLEVVEEAERLLGYSPEAFFTIHADKEDGTPLGGGSLAPPFPSPITVRNALARYADLLNSPKKSALVALATYASDSTEADRLRFLASPAGKDEYAQWVVASQRSLLEVMAEFPSAKPPLGVFFAAVAPRLQPRYYSISSSPSMAPTRIHVTCALVHEKTPAGRVHKGVCSTWIKNAIPSEETKDCSWAPVFVRQSNFKLPADPSVPVIMIGPGTGLAPFRGFLQERLSQKQSGAELGRSVFFFGCRNSKMDFIYEDELNTFLEEGALSELVLAFSREGPTKEYVQHKMSQKASEIWDMISQGGYIYVCGDAKGMARDVHRVLHTIVQEQGSLDSSKAESFVKSLQTEGRYLRDVW

SEQ ID NO: 40 - DNA, 27 bp, synthetic construct
  misc_feature 1..27: note = Synthetic HA epitope tag polypeptide
  source 1..27: mol_type = other DNA
tacccatacgacgttccagactacgcc

SEQ ID NO: 41 - protein, 9 aa, synthetic construct
  REGION 1..9: note = Synthetic HA epitope tag polypeptide
YPYDVPDYA

SEQ ID NO: 42 - DNA, 2169 bp, Oryza sativa
  source 1..2169: mol_type = genomic DNA
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggaagttctgttccaggggcccctgggatccccgatgctgccgccgtcgccgccggggtggccggtgatcgggcacctccacctcatgtccggcatgccgcaccacgcgctggccgagctggcgcgcaccatgcgcgcgccgctgttccggatgcggctggggagcgtgccggcggtggtgatctccaagccggacctcgcccgcgccgcgctcaccaccaacgacgccgcgctggcgtcgcggccgcacctgctctccggccagttcctgtcgttcggctgctccgacgtgacgttcgcgccggcggggccgtaccaccggatggcgcgccgcgtggtggtgtcggagctcctgtcggcgcgtcgcgtcgccacgtacggcgccgtcagggtcaaggagctccgccgcctgctcgcgcacctcaccaagaacacctcgccggcgaagcccgtcgacctcagcgagtgcttcctcaacctcgccaacgacgtgctctgccgcgtcgcgttcggccgccggttcccgcacggcgagggcgacaagctcggcgcggtgctcgccgaggcgcaggacctcttcgccgggttcaccatcggcgacttcttccccgagctcgagcccgtcgccagcaccgtcaccggactccgccgccgcctcaagaagtgcctcgccgacctccgcgaggcctgcgacgtgatcgtggacgaacacatcagcggcaaccgccagcgcatccccggcgaccgcgacgaggacttcgtcgacgtcctcctccgcgtccagaaatcccccgacctcgaggtccccctaaccgacgacaatctcaaggccctcgtcctggacatgttcgtcgccggcacggacaccacgttcgcgacgctggagtgggtgatgacggagctagtccgccacccacggatcctcaagaaggcgcaggaggaggtccggcgagtcgtcggcgacagcggccgcgtcgaggagtcccacctcggcgagctccactacatgcgcgccatcatcaaggagacgttccggctgcacccggcggtgccgttgctagtgccgcgcgagtccgtcgcgccgtgcacgctgggcggctacgacatcccggcgaggacgcgggtgttcatcaacacgttcgccatggggcgcgacccggagatctgggacaacccgctggagtactcgccggagaggttcgagagcgccggcggcggcggcgagatcgacctcaaggacccggactacaagctgctgccgttcggcggcgggcggcgagggtgccccggctacacgttcgcgctcgccaccgtgcaggtgtcgctcgccagcttgctctaccacttcgagtgggcgctgcccgccggcgtgcgcgccgaggacgtcaacctcgacgagacgttcggcctcgccacgaggaagaaggagccgctcttcgtcgccgtcaggaagagcgacgcgtacgagtttaagggagaggagcttagtgaggtttacccatactga

SEQ ID NO: 43 - protein, 722 aa, Oryza sativa
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGSPMLPPSPPGWPVIGHLHLMSGMPHHALAELARTMRAPLFRMRLGSVPAVVISKPDLARAALTTNDAALASRPHLLSGQFLSFGCSDVTFAPAGPYHRMARRVVVSELLSARRVATYGAVRVKELRRLLAHLTKNTSPAKPVDLSECFLNLANDVLCRVAFGRRFPHGEGDKLGAVLAEAQDLFAGFTIGDFFPELEPVASTVTGLRRRLKKCLADLREACDVIVDEHISGNRQRIPGDRDEDFVDVLLRVQKSPDLEVPLTDDNLKALVLDMFVAGTDTTFATLEWVMTELVRHPRILKKAQEEVRRVVGDSGRVEESHLGELHYMRAIIKETFRLHPAVPLLVPRESVAPCTLGGYDIPARTRVFINTFAMGRDPEIWDNPLEYSPERFESAGGGGEIDLKDPDYKLLPFGGGRRGCPGYTFALATVQVSLASLLYHFEWALPAGVRAEDVNLDETFGLATRKKEPLFVAVRKSDAYEFKGEELSEVYPY

SEQ ID NO: 44 - DNA, 3781 bp, synthetic construct
  misc_feature 1..3781: note = pCDFDuet-1 vector
  source 1..3781: mol_type = other DNA
ggggaattgtgagcggataacaattcccctgtagaaataattttgtttaactttaataaggagatataccatgggcagcagccatcaccatcatcaccacagccaggatccgaattcgagctcggcgcgcctgcaggtcgacaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggcagatctcaattggatatcggccggccacgcgatcgctgacgtcggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactata

(19) United States
(12) Patent Application Publication
(10) Pub. No.: US 2025/0100967 A1
(43) Pub. Date: Mar. 27, 2025

(54) HYDROXYLATED PSILOCYBIN DERIVATIVES AND METHODS OF USING

(71) Applicant: Enveric Biosciences Canada Inc., Calgary (CA)

(72) Inventors: Jillian M. Hagel, Calgary (CA); Peter J. Facchini, Calgary (CA); Chang-Chun Ling, Calgary (CA)

(21) Appl. No.: 18/971,774

(22) Filed: Dec. 6, 2024

Related U.S. Application Data
(63) Continuation of application No. 17/903,103, filed on Sep. 6, 2022, now Pat. No. 12,187,679.
Application No. 17/903,103 is a continuation of application No. PCT/CA2021/051210, filed on Sep. 1, 2021.
(60) Provisional application No. 63/073,076, filed on Sep. 1, 2020.

(51) Int. Cl. C07D 209/16

(52) U.S. Cl. CPC C07D 209/16

(57) ABSTRACT

Disclosed are novel hydroxylated psilocybin derivative compounds and pharmaceutical and recreational drug formulations containing the same. The compounds may be produced synthetically or biosynthetically.

### RELATED APPLICATION

This application is a continuation of U.S. patent application Ser. No. 17/903,103 filed Sep. 6, 2022, which is a continuation of PCT Application No. PCT/CA2021/051210 filed Sep. 1, 2021, which claims the benefit of U.S. Provisional Application No. 63/073,076 filed Sep. 1, 2020; the entire contents of which are hereby incorporated by reference.

### INCORPORATION OF SEQUENCE LISTING

A computer readable form of the Sequence Listing “29664-P62367US03_SequenceListing.xml” (93,886 bytes), submitted via Patent Center and created on Nov. 27, 2024, is herein incorporated by reference.

### FIELD OF THE DISCLOSURE

The compositions and methods disclosed herein relate to a chemical compound known as psilocybin. Furthermore, the compositions and methods disclosed herein relate in particular to hydroxylated forms of psilocybin.

### BACKGROUND OF THE DISCLOSURE

The following paragraphs are provided by way of background to the present disclosure. They are not however an admission that anything discussed therein is prior art or part of the knowledge of a person of skill in the art.

The biochemical pathways in the cells of living organisms may be classified as being part of primary metabolism, or as being part of secondary metabolism. Pathways that are part of a cell's primary metabolism are involved in catabolism for energy production or in anabolism for building block production for the cell. Secondary metabolites, on the other hand, are produced by the cell without having an obvious anabolic or catabolic function. It has long been recognized that secondary metabolites can be useful in many respects, including as therapeutic compounds.

Psilocybin, for example, is a secondary metabolite that is naturally produced by certain mushrooms which taxonomically can be classified as belonging the Basidiomycota division of the fungi kingdom. Mushroom species which can produce psilocybin include species belonging to the genus Psilocybe, such as Psilocybe azurescens, Psilocybe semilanceata, Psilocybe serbica, Psilocybe mexicana, and Psilocybe cyanescens, for example. The interest of the art in psilocybin is well established. Thus, for example, psilocybin is a psychoactive compound and is therefore used as a recreational drug. Furthermore, psilocybin is used as a research tool in behavioral and neuro-imaging studies in psychotic disorders, and has been evaluated for its clinical potential in the treatment of mental health conditions (Daniel, J. et al. Mental Health Clin/, 2017; 7 (1): 24-28), including to treat anxiety in terminal cancer patients (Grob, C. et al. Arch. Gen. Psychiatry, 2011, 68 (1) 71-78) and to alleviate symptoms of treatment-resistant depression (Cathart-Harris, R. L. et al. Lancet Psychiatry, 2016, 3:619-627).

Although the toxicity of psilocybin is low, adverse side effects, including, for example, panic attacks, paranoia and psychotic states, sometimes together or individually referred to as “a bad trip”, are not infrequently experienced by recreational psilocybin users.

There exists therefore a need in the art for improved psilocybin compounds.

### SUMMARY OF THE DISCLOSURE

The following paragraphs are intended to introduce the reader to the more detailed description, not to define or limit the claimed subject matter of the present disclosure.

In one aspect, the present disclosure relates to psilocybin and derivative compounds.

In another aspect, the present disclosure relates to hydroxylated psilocybin derivative compounds and methods of making and using these compounds.

Accordingly, in one aspect, the present disclosure provides, in at least one embodiment, in accordance with the teachings herein, a chemical compound or salt thereof having formula (I):

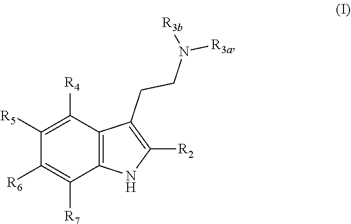

wherein at least one of R₂, R₄, R₅, R₆, or R₇ is a hydroxy group, and wherein each non-hydroxylated R₂, R₅, R₆, or R is a hydrogen atom or an alkyl group or O-alkyl group, wherein R₄ when it is not hydroxylated is a hydrogen atom, an alkyl group or a phosphate group, and wherein R_{3A} and R_{3B} are a hydrogen atom, an alkyl group, a cycloalkyl group, an aryl group, an alkaryl group or an acyl group.

In at least one embodiment, in an aspect, R₂ can be a hydroxy group, R₅, R₆ and R₇ can each be a hydrogen atom or an alkyl group or O-alkyl group, and R₄ can be a phosphate group, a hydrogen atom or an alkyl group or O-alkyl group.

In at least one embodiment, in an aspect, R₄ can be a hydroxy group and R₂, R₅, R₆ and R₇ can each be a hydrogen atom or an alkyl group or O-alkyl group.

In at least one embodiment, in an aspect, R₅ can be a hydroxy group, R₂, R₆ and R₇ can each be a hydrogen atom or an alkyl group or O-alkyl group, and R₄ can be a phosphate group, a hydrogen atom or an alkyl group or O-alkyl group.

In at least one embodiment, in an aspect, R₆ can be a hydroxy group, R₂, R₅ and R₇ can each be a hydrogen atom or an alkyl group or O-alkyl group, and R₄ can be a phosphate group, a hydrogen atom or an alkyl group or O-alkyl group.

In at least one embodiment, in an aspect, R₇ can be a hydroxy group, R₂, R₅ and R₆ can each be a hydrogen atom or an alkyl group or O-alkyl group, and R₄ can be a phosphate group, a hydrogen atom or an alkyl group or O-alkyl group.

In at least one embodiment, in an aspect, at least two of R₂, R₄, R₅, R₆ or R₇ can be a hydroxy group.

In at least one embodiment, in an aspect, R₂ and R₄ can be a hydroxy group, and R₅, R₆ and R₇ can be a hydrogen atom or an alkyl group or O-alkyl group.

In at least one embodiment, in an aspect, R₂ and R₅ can be a hydroxy group, R₆ and R₇ can be a hydrogen atom, and R₄ can be a hydrogen atom, an alkyl group or a phosphate group.

In at least one embodiment, in an aspect, R₂ and R₆ can be a hydroxy group, R₅ and R₇ can be a hydrogen atom or an alkyl group or O-alkyl group, and R₄ can be a hydrogen atom, an alkyl group or a phosphate group.

In at least one embodiment, in an aspect, R₂ and R₇ can be a hydroxy group, R₅ and R₆ can be a hydrogen atom or an alkyl group or O-alkyl group, and R₄ can be a hydrogen atom, an alkyl group an alkyl group or a phosphate group.

In at least one embodiment, in an aspect, R₄ and R₅ can be a hydroxy group, and R₂, R₆ and R₇ can be a hydrogen atom or an alkyl group or O-alkyl group.

In at least one embodiment, in an aspect, R₄ and R₆ can be a hydroxy group, and R₂, R₅ and R₇ can be a hydrogen atom or an alkyl group or O-alkyl group.

In at least one embodiment, in an aspect, R₄ and R₇ can be a hydroxy group and R₂, R₅ and R₆ can be a hydrogen atom or an alkyl group or O-alkyl group.

In at least one embodiment, in an aspect, R₅ and R₆ can be a hydroxy group, R₂ and R₇ can be a hydrogen atom or an alkyl group or O-alkyl group, and R₄ can be a hydrogen atom, an alkyl group or a phosphate group.

In at least one embodiment, in an aspect, R₅ and R₇ can be a hydroxy group, R₂ and R₆ can be a hydrogen atom or an alkyl group or O-alkyl group, and R₄ can be a hydrogen atom, an alkyl group or a phosphate group.

In at least one embodiment, in an aspect, R₆ and R₇ can be a hydroxy group, R₂ and R₅ can be a hydrogen atom or an alkyl group or O-alkyl group, and R₄ can be a hydrogen atom, an alkyl group or a phosphate group.

In at least one embodiment, in an aspect, R₄ when it is not hydroxylated can be a hydrogen atom.

In at least one embodiment, in an aspect, R₄ when it is not hydroxylated can be an alkyl group.

In at least one embodiment, in an aspect, R₄ when it is not hydroxylated can be an O-alkyl group.

In at least one embodiment, in an aspect, R₄ when it is not hydroxylated can be a phosphate group.

In at least one embodiment, in an aspect, three, four or all five of R₂, R₄, R₅, R₆ or R₇ can be a hydroxy group.

In at least one embodiment, in an aspect, the chemical compound can be selected from the group consisting of compounds having formulas (III); (IV); (V); (VI); (VII); (VIII); (IX); (X); (XI); (XII); (XIII); (XIV); (XV); (XVI); (XVII); (XVIII); (XIX); (XX); (XXI); (XXII); (XXIII); (XXIV); (XXV); (XXVI); (XXVII); (XXVIII); (XXIX); (XXX); (XXXI); and (XXXII):

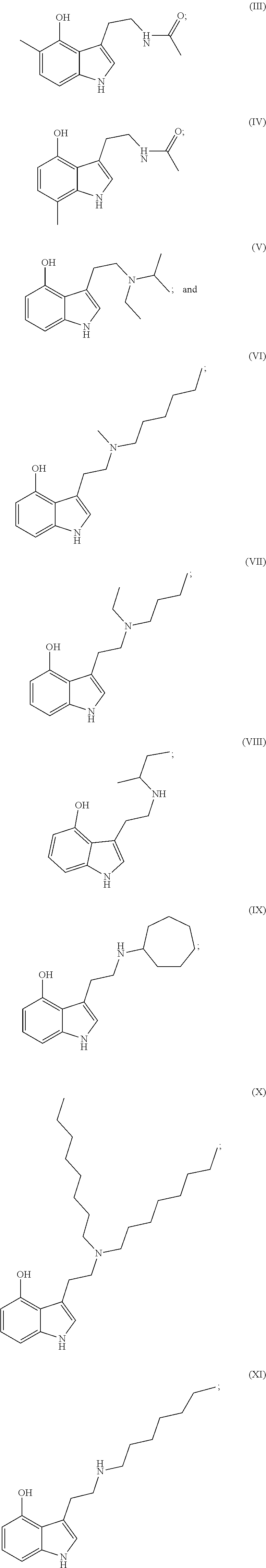

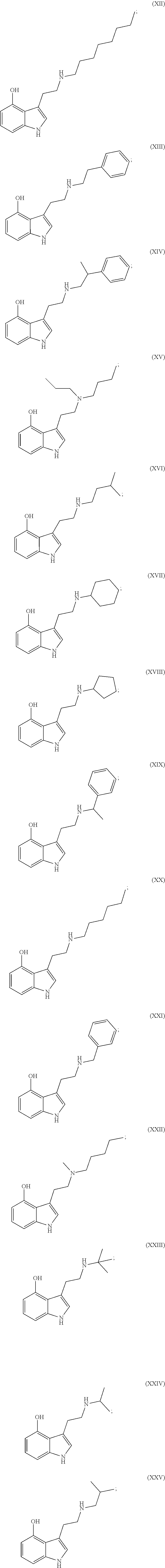

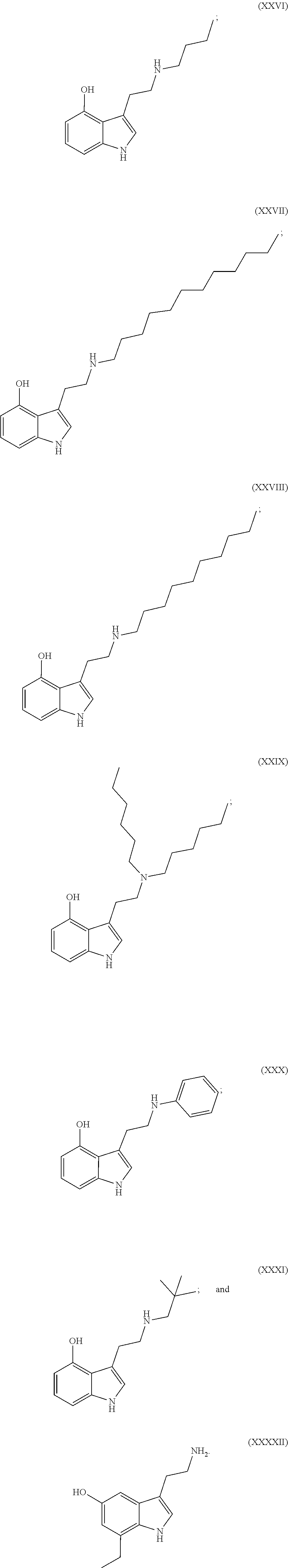

In another aspect, the present disclosure relates to pharmaceutical and recreational drug formulations comprising hydroxylated psilocybin derivatives. Accordingly, in one aspect, the present disclosure provides, in at least one embodiment, a pharmaceutical or recreational drug formulation comprising an effective amount of a chemical compound or salt thereof having formula (I):

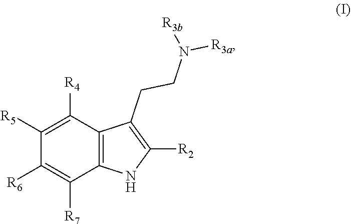

wherein at least one of R₂, R₄, R₅, R₆, or R₇ is a hydroxy group, and wherein each non-hydroxylated R₂, R₅, R₆, or R₇ is a hydrogen atom or an alkyl group or O-alkyl group, wherein R₄ when it is not hydroxylated is a hydrogen atom, an alkyl group or a phosphate group, and wherein R_{3A} and R_{3B} are a hydrogen atom, an alkyl group, a cycloalkyl group, an aryl group, an alkaryl group, or an acyl group, together with a pharmaceutically acceptable carrier, diluent or excipient.

In another aspect, the present disclosure relates to methods of treatment of psychiatric disorders. Accordingly, the present disclosure further provides, in at least one embodiment, a method for treating a psychiatric disorder, the method comprising administering to a subject in need thereof a pharmaceutical formulation comprising a chemical compound or salt thereof having formula (I):

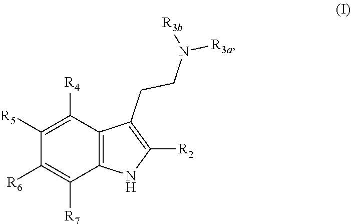

wherein at least one of R₂, R₄, R₅, R₆, or R₇ is a hydroxy group, and wherein each non-hydroxylated R₂, R₅, R₆, or R₇ is a hydrogen atom or an alkyl group or O-alkyl group, wherein R₄ when it is not hydroxylated is a hydrogen atom, an alkyl group or a phosphate group, and wherein R_{3A} and R_{3B} are a hydrogen atom, an alkyl group, a cycloalkyl group, an aryl group, an alkaryl group, or an acyl group, wherein the pharmaceutical formulation is administered in an effective amount to treat the psychiatric disorder in the subject.

In at least one embodiment, in an aspect, the disorder can be a 5-HT_{2A} receptor mediated disorder.

In at least one embodiment, in an aspect, a dose can be administered of about 0.001 mg to about 5,000 mg.

In another aspect, the present disclosure relates to methods of making hydroxylated psilocybin derivatives. Accordingly, in one aspect, the present disclosure provides, in at least one embodiment, a method of making a hydroxylated psilocybin derivative the method comprising:
(a) contacting a psilocybin precursor compound with a host cell comprising a psilocybin biosynthetic enzyme complement; and
(b) growing the host cell to produce a hydroxylated psilocybin derivative compound having the formula (I):

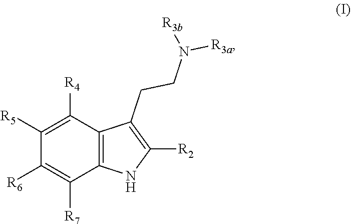

wherein, at least one of R₂, R₄, R₅, R₆, or R₇ is a hydroxy group, and wherein each non-hydroxylated R₂, R₅, R₆, or R₇ is a hydrogen atom or an alkyl group or O-alkyl group, wherein R₄ when it is not hydroxylated is a phosphate group, a hydrogen atom, or an alkyl group, and wherein R_{3A} and R_{3B} are a hydrogen atom, an alkyl group, a cycloalkyl group, an aryl group, an alkaryl group, or an acyl group.

In at least one embodiment, in an aspect, the psilocybin precursor compound can be a hydroxy-derivative psilocybin derivative compound selected from hydroxylated indole or an alkyl or O-alkyl derivative thereof, hydroxylated tryptophan or an alkyl or O-alkyl derivative thereof, and hydroxylated tryptamine or an alkyl or O-alkyl derivative thereof and the hydroxy-derivative psilocybin derivative compound is included in a medium to grow the host cell.

In at least one embodiment, in an aspect, the psilocybin precursor compound can be a hydroxy-derivative psilocybin derivative compound selected from hydroxylated indole or an alkyl or O-alkyl derivative thereof and hydroxylated tryptophan or an alkyl or O-alkyl derivative thereof, and the psilocybin biosynthetic enzyme complement can comprise at least one of TrpB and PsiD wherein TrpB is encoded by a nucleic acid sequence selected from:
(a) SEQ.ID NO: 11 or SEQ.ID NO: 26;
(b) a nucleic acid sequence that is substantially identical to any one of the nucleic acid sequences of (a);
(c) a nucleic acid sequence that is substantially identical to any one of the nucleic acid sequences of (a) but for the degeneration of the genetic code;
(d) a nucleic acid sequence that is complementary to any one of the nucleic acid sequences of (a);
(e) a nucleic acid sequence encoding a polypeptide having any one of the amino acid sequences set forth in SEQ. ID NO: 12 and SEQ. ID NO: 27;
(f) a nucleic acid sequence that encodes a functional variant of any one of the amino acid sequences set forth in SEQ.ID NO: 12 and SEQ.ID NO: 27; and
(g) a nucleic acid sequence that hybridizes under stringent conditions to any one of the nucleic acid sequences set forth in (a), (b), (c), (d), (e) or (f); and
   wherein the PsiD is encoded by a nucleic acid sequence selected from:
(a) SEQ.ID NO: 1, SEQ.ID NO: 9 or SEQ.ID NO: 31;
(b) a nucleic acid sequence that is substantially identical to any one of the nucleic acid sequences of (a);
(c) a nucleic acid sequence that is substantially identical to any one of the nucleic acid sequences of (a) but for the degeneration of the genetic code;
(d) a nucleic acid sequence that is complementary to any one of the nucleic acid sequences of (a);
(e) a nucleic acid sequence encoding a polypeptide having any one of the amino acid sequences set forth in SEQ.ID NO: 2, SEQ.ID NO: 10 and SEQ.ID NO: 32;
(f) a nucleic acid sequence that encodes a functional variant of any one of the amino acid sequences set forth in SEQ.ID NO: 2, SEQ.ID NO: 10 and SEQ.ID NO: 32; and
(g) a nucleic acid sequence that hybridizes under stringent conditions to any one of the nucleic acid sequences set forth in (a), (b), (c), (d), (e) or (f).

In at least one embodiment, in an aspect, the psilocybin precursor compound can be a hydroxy-derivative psilocybin derivative compound selected from hydroxylated tryptamine or an alkyl or O-alkyl derivative thereof and hydroxylated tryptophan or an alkyl or O-alkyl derivative thereof, and the psilocybin biosynthetic enzyme complement can comprise at least one of PsiH and PsiD wherein PsiH is encoded by a nucleic acid sequence selected from:
(a) SEQ.ID NO: 3;
(b) a nucleic acid sequence that is substantially identical to SEQ.ID NO: 3;
(c) a nucleic acid sequence that is substantially identical to SEQ.ID NO: 3 but for the degeneration of the genetic code;
(d) a nucleic acid sequence that is complementary to SEQ.ID NO: 3;
(e) a nucleic acid sequence encoding a polypeptide having the amino acid sequences set forth in SEQ.ID NO: 4;
(f) a nucleic acid sequence that encodes a functional variant of any the amino acid sequence set forth in SEQ.ID NO: 4; and
(g) a nucleic acid sequence that hybridizes under stringent conditions to any one of the nucleic acid sequences set forth in (a), (b), (c), (d), (e) or (f); and
   wherein the PsiD is encoded by a nucleic acid sequence selected from:
(a) SEQ.ID NO: 1, SEQ. ID NO: 9 or SEQ.ID NO: 31;
(b) a nucleic acid sequence that is substantially identical to any one of the nucleic acid sequences of (a);
(c) a nucleic acid sequence that is substantially identical to any one of the nucleic acid sequences of (a) but for the degeneration of the genetic code;
(d) a nucleic acid sequence that is complementary to any one of the nucleic acid sequences of (a);
(e) a nucleic acid sequence encoding a polypeptide having any one of the amino acid sequences set forth in SEQ.ID NO: 2, SEQ.ID NO: 10 and SEQ.ID NO: 32;
(f) a nucleic acid sequence that encodes a functional variant of any one of the amino acid sequences set forth in SEQ.ID NO: 2, SEQ.ID NO: 10 and SEQ.ID NO: 32; and
(g) a nucleic acid sequence that hybridizes under stringent conditions to any one of the nucleic acid sequences set forth in (a), (b), (c), (d), (e) or (f).

In at least one embodiment, in aspect, the psilocybin biosynthetic enzyme complement can further comprise an acetyl transferase wherein the acetyl transferase is encoded by a nucleic acid sequence selected from:
(a) SEQ.ID NO: 24;
(b) a nucleic acid sequence that is substantially identical to SEQ.ID NO: 24;
(c) a nucleic acid sequence that is substantially identical to SEQ.ID NO: 24 but for the degeneration of the genetic code;
(d) a nucleic acid sequence that is complementary to SEQ. ID NO: 24;
(e) a nucleic acid sequence encoding a polypeptide having the amino acid sequences set forth in SEQ.ID NO: 25;
(f) a nucleic acid sequence that encodes a functional variant of the amino acid sequences set forth in SEQ. ID NO: 25; and
(g) a nucleic acid sequence that hybridizes under stringent conditions to any one of the nucleic acid sequences set forth in (a), (b), (c), (d), (e) or (f).

In at least one embodiment, in an aspect, the psilocybin biosynthetic enzyme complement can further comprise at least one enzyme encoded by a nucleic acid selected from:
(a) SEQ.ID NO: 5 and SEQ.ID NO: 7;
(b) a nucleic acid sequence that is substantially identical to any one of the nucleic acid sequences of (a);
(c) a nucleic acid sequence that is substantially identical to any one of the nucleic acid sequences of (a) but for the degeneration of the genetic code;
(d) a nucleic acid sequence that is complementary to any one of the nucleic acid sequences of (a);
(e) a nucleic acid sequence encoding a polypeptide having any one of the amino acid sequences set forth in SEQ. ID NO: 6 and SEQ. ID NO: 8;
(f) a nucleic acid sequence that encodes a functional variant of any one of the amino acid sequences set forth in SEQ. ID NO: 6 and SEQ. ID NO: 8; and
(g) a nucleic acid sequence that hybridizes under stringent conditions to any one of the nucleic acid sequences set forth in (a), (b), (c), (d), (e) or (f).

In at least one embodiment, in an aspect, the psilocybin precursor compound can be selected from 4-hydroxy-5 methyl indole and 4-hydroxy-7-methyl-indole, and the hydroxylated psilocybin derivative can be a compound having the chemical formula (III) or (IV):

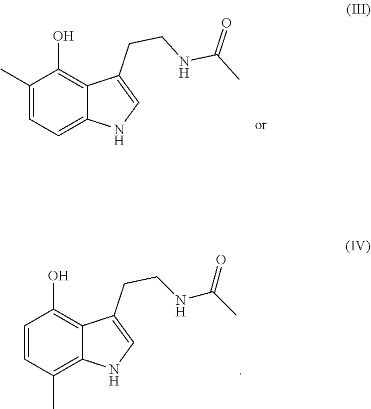 [SMILES: CC(=O)NCCc1c[nH]c2ccc(C)c(O)c12 ; CC(=O)NCCc1c[nH]c2c(C)ccc(O)c12]

In at least one embodiment, in an aspect, the psilocybin precursor compound can be a hydroxy-derivative psilocybin derivative compound selected from a hydroxylated indole or an alkyl or O-alkyl derivative thereof, a hydroxylated tryptophan or an alkyl or O-alkyl derivative thereof, or a hydroxylated tryptamine or an alkyl or O-alkyl derivative which is formed by contacting the host cell with a non-hydroxy-derivative psilocybin precursor compound, wherein the non-hydroxy-derivative psilocybin precursor compound is selected from indole or an alkyl or O-alkyl derivative thereof, tryptophan or an alkyl or O-alkyl derivative or tryptamine or an alkyl or O-alkyl derivative thereof, the host cell further comprising a hydroxylase capable of hydroxylating the non-hydroxylated psilocybin compound and forming the hydroxy-derivative psilocybin precursor compound.

In at least one embodiment, in an aspect, the hydroxylase can be encoded by a nucleic acid selected from:
(a) SEQ.ID NO: 13, SEQ.ID NO: 15, SEQ. ID NO: 17, SEQ.ID NO: 19, SEQ. ID NO: 38 and SEQ. ID NO: 42;
(b) a nucleic acid sequence that is substantially identical to any one of the nucleic acid sequences of (a);
(c) a nucleic acid sequence that is substantially identical to any one of the nucleic acid sequences of (a) but for the degeneration of the genetic code;
(d) a nucleic acid sequence that is complementary to any one of the nucleic acid sequences of (a);
(e) a nucleic acid sequence encoding a polypeptide having any one of the amino acid sequences set forth in SEQ.ID NO: 14, SEQ.ID NO: 16, SEQ.ID NO: 18, SEQ. ID NO: 39, and SEQ. ID NO: 43;
(f) a nucleic acid sequence that encodes a functional variant of any one of the amino acid sequences set forth in SEQ.ID NO: 14, SEQ. ID NO: 16, SEQ.ID NO: 18, SEQ. ID NO: 39 and SEQ. ID NO: 43; and
(g) a nucleic acid sequence that hybridizes under stringent conditions to any one of the nucleic acid sequences set forth in (a), (b), (c), (d), I or (f).

In at least one embodiment, the biosynthetic enzyme complement can further comprise at least one of TrpB and PsiD wherein TrpB is encoded by a nucleic acid sequence selected from:
(a) SEQ.ID NO: 11 or SEQ.ID NO: 26;
(b) a nucleic acid sequence that is substantially identical to any one of the nucleic acid sequences of (a);
(c) a nucleic acid sequence that is substantially identical to any one of the nucleic acid sequences of (a) but for the degeneration of the genetic code;
(d) a nucleic acid sequence that is complementary to any one of the nucleic acid sequences of (a);
(e) a nucleic acid sequence encoding a polypeptide having any one of the amino acid sequences set forth in SEQ.ID NO: 12 and SEQ. ID NO: 27;
(f) a nucleic acid sequence that encodes a functional variant of any one of the amino acid sequences set forth in SEQ. ID NO: 12 and SEQ.ID NO: 27; and
(g) a nucleic acid sequence that hybridizes under stringent conditions to any one of the nucleic acid sequences set forth in (a), (b), (c), (d), (e) or (f); and
   wherein the PsiD is encoded by a nucleic acid sequence selected from:
(a) SEQ.ID NO: 1, SEQ.ID NO: 9 or SEQ.ID NO: 31;
(b) a nucleic acid sequence that is substantially identical to any one of the nucleic acid sequences of (a);
(c) a nucleic acid sequence that is substantially identical to any one of the nucleic acid sequences of (a) but for the degeneration of the genetic code;
(d) a nucleic acid sequence that is complementary to any one of the nucleic acid sequences of (a);
(e) a nucleic acid sequence encoding a polypeptide having any one of the amino acid sequences set forth in SEQ.ID NO: 2, SEQ.ID NO: 10 and SEQ.ID NO: 32;
(f) a nucleic acid sequence that encodes a functional variant of any one of the amino acid sequences set forth in SEQ.ID NO: 2, SEQ.ID NO: 10 and SEQ.ID NO: 32; and
(g) a nucleic acid sequence that hybridizes under stringent conditions to any one of the nucleic acid sequences set forth in (a), (b), (c), (d), (e) or (f).

In at least one embodiment, in an aspect, the psilocybin biosynthetic enzyme complement can further comprise at least one of PsiH and PsiD wherein PsiH is encoded by a nucleic acid sequence selected from:
(a) SEQ.ID NO: 3;
(b) a nucleic acid sequence that is substantially identical to SEQ.ID NO: 3;
(c) a nucleic acid sequence that is substantially identical to SEQ.ID NO: 3 but for the degeneration of the genetic code;
(d) a nucleic acid sequence that is complementary to SEQ. ID NO: 3;
(e) a nucleic acid sequence encoding a polypeptide having the amino acid sequences set forth in SEQ.ID NO: 4;
(f) a nucleic acid sequence that encodes a functional variant of any the amino acid sequence set forth in SEQ. ID NO: 4; and
(g) a nucleic acid sequence that hybridizes under stringent conditions to any one of the nucleic acid sequences set forth in (a), (b), (c), (d), (e) or (f); and
   wherein the PsiD is encoded by a nucleic acid sequence selected from:
(a) SEQ.ID NO: 1, SEQ. ID NO: 9 or SEQ. ID NO: 31;
(b) a nucleic acid sequence that is substantially identical to any one of the nucleic acid sequences of (a);
(c) a nucleic acid sequence that is substantially identical to any one of the nucleic acid sequences of (a) but for the degeneration of the genetic code;
(d) a nucleic acid sequence that is complementary to any one of the nucleic acid sequences of (a);
(e) a nucleic acid sequence encoding a polypeptide having any one of the amino acid sequences set forth in SEQ.ID NO: 2, SEQ.ID NO: 10 and SEQ.ID NO: 32;
(f) a nucleic acid sequence that encodes a functional variant of any one of the amino acid sequences set forth in SEQ.ID NO: 2, SEQ. ID NO: 10 and SEQ.ID NO: 32; and
(g) a nucleic acid sequence that hybridizes under stringent conditions to any one of the nucleic acid sequences set forth in (a), (b), (c), (d), (e) or (f).

In at least one embodiment, in aspect, the psilocybin biosynthetic enzyme complement can further comprise an acetyl transferase wherein the acetyl transferase is encoded by a nucleic acid sequence selected from:
(a) SEQ.ID NO: 24;
(b) a nucleic acid sequence that is substantially identical to SEQ.ID NO: 24;
(c) a nucleic acid sequence that is substantially identical to SEQ.ID NO: 24 but for the degeneration of the genetic code;
(d) a nucleic acid sequence that is complementary to SEQ.ID NO: 24;
(e) a nucleic acid sequence encoding a polypeptide having the amino acid sequences set forth in SEQ.ID NO: 25;
(f) a nucleic acid sequence that encodes a functional variant of the amino acid sequences set forth in SEQ. ID NO: 25; and
(g) a nucleic acid sequence that hybridizes under stringent conditions to any one of the nucleic acid sequences set forth in (a), (b), (c), (d), (e) or (f).

In at least one embodiment, in an aspect, the psilocybin precursor compound can be 7-ethyl indole and the hydroxylated psilocybin derivative can be a compound having the chemical formula (XXXII):

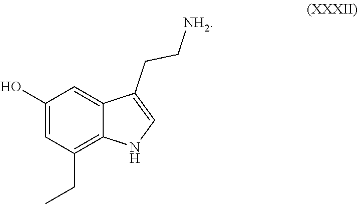

In at least one embodiment, in an aspect, the method can further include a step comprising isolating the hydroxylated psilocybin derivative compound.

In at least one embodiment, in an aspect, the host cell can be a microbial cell.

In at least one embodiment, in an aspect, the host cell can be a bacterial cell or a yeast cell.

In another aspect, the present disclosure provides, in at least one embodiment, a method for modulating a 5-HT_{2A} receptor, the method comprising contacting a 5-HT_{2A} receptor with a chemical compound or salt thereof having formula (I)

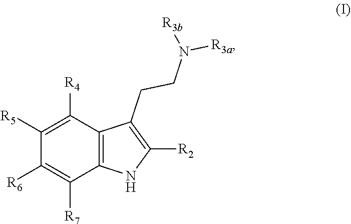

wherein at least one of R₂, R₄, R₅, R₆, or R₇ is a hydroxy group, and wherein each non-hydroxylated R₂, R₅, R₆, or R₇ is a hydrogen atom or an alkyl group or O-alkyl group, wherein R₄ when it is not hydroxylated is a hydrogen atom, an alkyl group or O-alkyl group or a phosphate group, and wherein R_{3A} and R_{3B} are a hydrogen atom, an alkyl group, a cycloalkyl group, an aryl group, an alkaryl group, or an acyl group under reaction conditions sufficient to thereby modulate receptor activity.

In some embodiments, in an aspect, the reaction conditions can be in vitro reaction conditions.

In some embodiments, in an aspect, the reaction conditions can be in vivo reaction conditions.

In another aspect, the present disclosure provides, in at least one embodiment, a use of a chemical compound having the formula (I):

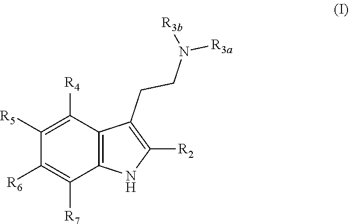

wherein, at least one of R₂, R₄, R₅, R₆, or R₇ is a hydroxy group, and wherein each non-hydroxylated R₂, R₅, R₆, or R₇ is a hydrogen atom or an alkyl group or O-alkyl group, wherein R₄ when it is not hydroxylated is a phosphate group, a hydrogen atom, or an alkyl group and wherein R_{3A} and R_{3B} are a hydrogen atom, an alkyl group, a cycloalkyl group, an alkyl group, an alkaryl group or an acyl group, in the manufacture of a pharmaceutical or recreational drug formulation.

In at least one embodiment, in an aspect, the manufacture can comprise formulating the chemical compound with an excipient, diluent or carrier.

In at least one embodiment, in an aspect, the manufacture can further include a step comprising derivatizing the chemical compound having the formula (I) by substituting the hydroxy group with another group or an atom.

In another aspect, the present disclosure provides, in at least one embodiment, a use of a chemical compound having the formula (I):

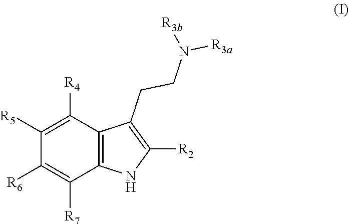

wherein, at least one of R₂, R₄, R₅, R₆, or R₇ is a hydroxy group, and wherein each non-hydroxylated R₂, R₅, R₆, or R₇ is a hydrogen atom or an alkyl group or O-alkyl group, wherein R₄ when it is not hydroxylated is a phosphate group, a hydrogen atom or an alkyl group or O-alkyl group, and wherein RA and R_{3B} are a hydrogen atom, an alkyl group, a cycloalkyl group, an alkyl group, an alkaryl group or an acyl group, together with a diluent, carrier, or excipient as a pharmaceutical or recreational drug formulation.

Other features and advantages will become apparent from the following detailed description. It should be understood, however, that the detailed description, while indicating preferred implementations of the disclosure, are given by way of illustration only, since various changes and modifications within the spirit and scope of the disclosure will become apparent to those of skill in the art from the detailed description.

### BRIEF DESCRIPTION OF THE DRAWINGS

The disclosure is in the hereinafter provided paragraphs described, by way of example, in relation to the attached figures. The figures provided herein are provided for a better understanding of the example embodiments and to show more clearly how the various embodiments may be carried into effect. The figures are not intended to limit the present disclosure.

FIG. 1 depicts the chemical structure of psilocybin.

FIG. 2 depicts a certain prototype structure of psilocybin and psilocybin derivative compounds, namely an indole. Certain carbon and nitrogen atoms may be referred to herein by reference to their position within the indole structure, i.e. N₁, C₂, C₃ etc. The pertinent atom numbering is shown.

FIGS. 3A, 3B, 3C, 3D, 3E, 3F, 3G, 3H, 3I, 3J, 3K, 3L, 3M, 3N, 3O, 3P and 3Q depict the chemical structures of certain example hydroxylated psilocybin derivative compounds, notably a 2-hydroxy psilocybin derivative (FIG. 3A); a 4-hydroxy derivative (FIG. 3B); a 5-hydroxy psilocybin derivative (FIG. 3C); a 6-hydroxy psilocybin derivative (FIG. 3D); a 4-phospho-7-hydroxy psilocybin derivative (FIG. 3E); a 2-hydroxy-4-phospho psilocybin derivative (FIG. 3F); a 4-phospho-5-hydroxy psilocybin derivative (FIG. 3G); a 4-phospho-6-hydroxy psilocybin derivative (FIG. 3H); a 4-phospho-7-hydroxy psilocybin derivative (FIG. 3I); a 2-hydroxy-4-methyl psilocybin derivative (FIG. 3J); a 4-ethyl-5-hydroxy psilocybin derivative (FIG. 3K); a 4-methyl-6-hydroxy psilocybin derivative (FIG. 3L); a 4-propyl-7-hydroxy psilocybin derivative (FIG. 3M); a 2-hydroxy-4-O-methyl psilocybin derivative (FIG. 3N); a 4-O-ethyl-5-hydroxy psilocybin derivative (FIG. 3O); a 4-O-methyl-6-hydroxy psilocybin derivative (FIG. 3P); and a 4-O-propyl-7-hydroxy psilocybin derivative (FIG. 3Q) It is noted that in each of FIGS. 3A, 3B, 3C, 3D, 3E, 3F, 3G, 3H, 3I, 3J, 3K, 3L, 3M, 3N, 3O, 3P and 3Q, R₃ₐ and R_{3b} can be a hydrogen atom, an alkyl group, a cycloalkyl group, an aryl group, an alkaryl group, or an acyl group.

FIGS. 4A, 4B, 4C, 4D, 4E, 4F, 4G, 4H, 4I and 4J depict the chemical structures of certain further example hydroxylated psilocybin derivative compounds, notably a 2,4-di-hydroxy psilocybin derivative (FIG. 4A); a 2,5-hydroxy psilocybin derivative (FIG. 4B); a 2,6-di-hydroxy-4-methyl psilocybin derivative (FIG. 4C); a 2,7-di-hydroxy-4-phospho psilocybin derivative (FIG. 4D); a 4,5-di-hydroxy psilocybin derivative (FIG. 4E); a 4,6-di-hydroxy psilocybin derivative (FIG. 4F); a 4,7-di-hydroxy psilocybin derivative (FIG. 4G); a 4-phospho-5,6-di-hydroxy psilocybin derivative (FIG. 4H) a 4-phospho-5,7-di-hydroxy psilocybin derivative (FIG. 4I); and a 6,7-di-hydroxy psilocybin derivative (FIG. 4J). It is noted that in each of FIGS. 4A, 4B, 4C, 4D, 4E, 4F, 4G, 4H, 4I and 4J R₃ₐ and R_{3b} can be a hydrogen atom, an alkyl group, a cycloalkyl group, an aryl group, an alkaryl group, or an acyl group.

FIGS. 5A, 5B, 5C, 5D, 5E, and 5F depict the chemical structures of certain further example hydroxylated psilocybin derivative compounds, notably a 2,4,5-tri-hydroxy psilocybin derivative (FIG. 5A); a 2-5,6-tri-hydroxy-4-methyl psilocybin derivative (FIG. 5B); a 2,5,7-tri-hydroxy psilocybin derivative (FIG. 5C); a 4,5,6-tri-hydroxy psilocybin derivative (FIG. 5D); a 4,5,7-tri-hydroxy psilocybin derivative (FIG. 5E); and a 4-phospho-5,6,7-tri-hydroxy psilocybin derivative (FIG. 5F). It is noted that in each of FIGS. 5A, 5B, 5C, 5D, 5E, and 5F R₃ₐ and R_{3b} can be a hydrogen atom, an alkyl group, a cycloalkyl group, an aryl group, an alkaryl group or an acyl group.

FIGS. 6A, 6B, 6C, 6D, and 6E depict the chemical structures of certain further example hydroxylated psilocybin derivative compounds, notably a 2,4,5,6-tetrhydroxy psilocybin derivative (FIG. 6A); a 4,5,6,7-tetra hydroxy psilocybin derivative (FIG. 6B); a 2,5,6-7-tetra-hydroxy-4-phospho psilocybin derivative (FIG. 6C); a 2,4,6,7-tetra hydroxy psilocybin derivative (FIG. 6D); and a 2,4,5,7-tetra-hydroxy psilocybin derivative (FIG. 6E). It is noted that in each of FIGS. 6A, 6B, 6C, 6D, and 6E R₃ₐ and R_{3b} can be a hydrogen atom, an alkyl group, a cycloalkyl group, an alkaryl group, an aryl group, or an acyl group.

FIG. 7 depicts a biosynthesis pathway for the synthesis of psilocybin.

FIGS. 8A, 8B, 8C, and 8D depict various graphs obtained in the performance of an experimental assay to evaluate the efficacy of an example hydroxylated psilocybin derivative having the chemical formula (IV) set forth herein, notably a cell viability assay (FIG. 8A), a serotonin positive allosteric modulation assay in +5-HT₂ₐ cells (FIG. 8B), a serotonin calcium flux assay (FIG. 8C), and control assays: serotonin response in-5-HT₂ₐ cells (FIG. 8D—panel A) and methanol response in +5-HT₂ₐ cells (FIG. 8D—panel B).

FIGS. 9A, 9B and 9C depict various graphs obtained in the performance of an experimental assay to evaluate the efficacy of an example hydroxylated psilocybin derivative having the chemical formula (III) set forth herein, notably a cell viability assay (FIG. 9A), a serotonin positive allosteric modulation assay in +5-HT₂ₐ cells (FIG. 9B), and a serotonin calcium flux assay (FIG. 9C).

FIG. 10 depicts a representation of mass spectrometry data in the form of a chromatogram, notably a chromatogram obtained in the performance of an experiment to synthesize an example hydroxylated psilocybin derivative compound having the chemical formula (XXXII) set forth herein.

FIG. 11 depicts an example process for chemically example synthesizing hydroxylated psilocybin compounds

FIG. 12 depicts a representation of mass spectrometry data in the form of a chromatogram, notably a chromatogram obtained in the performance of an experiment to synthesize an example hydroxylated psilocybin derivative compound having the chemical formula (V) set forth herein.

FIG. 13 depicts a representation of mass spectrometry data in the form of a mass spectrometry spectrum obtained in the performance of an experiment to identify a hydroxylated psilocybin derivative compound having the chemical formula (V) set forth herein.

FIG. 14 depicts a representation of mass spectrometry data in the form of a chromatogram, notably a chromatogram obtained in the performance of an experiment to synthesize an example hydroxylated psilocybin derivative compound having the chemical formula (IX) set forth herein.

FIGS. 15A and 15B depict a representation of mass spectrometry data in the form of a chromatogram, notably a chromatogram obtained in the performance of an experiment to synthesize an example hydroxylated psilocybin derivative compound having the chemical formula (X) set forth herein (FIG. 15A), and in the form of a mass spectrometry spectrum obtained in the performance of an experiment to identify a hydroxylated psilocybin derivative compound having the chemical formula (X) set forth herein (FIG. 15B).

FIGS. 16A and 16B depict a representation of mass spectrometry data in the form of a chromatogram, notably a chromatogram obtained in the performance of an experiment to synthesize an example hydroxylated psilocybin derivative compound having the chemical formula (XIII) set forth herein (FIG. 16A), and in the form of a mass spectrometry spectrum obtained in the performance of an experiment to identify a hydroxylated psilocybin derivative compound having the chemical formula (XIII) set forth herein (FIG. 16B).

FIGS. 17A and 17B depict a representation of mass spectrometry data in the form of a chromatogram, notably a chromatogram obtained in the performance of an experiment to synthesize an example hydroxylated psilocybin derivative compound having the chemical formula (XIV) set forth herein (FIG. 17A), and in the form of a mass spectrometry spectrum obtained in the performance of an experiment to identify a hydroxylated psilocybin derivative compound having the chemical formula (XIV) set forth herein (FIG. 17B).

FIGS. 18A and 18B depict a representation of mass spectrometry data in the form of a chromatogram, notably a chromatogram obtained in the performance of an experiment to synthesize an example hydroxylated psilocybin derivative compound having the chemical formula (XV) set forth herein (FIG. 18A), and in the form of a mass spectrometry spectrum obtained in the performance of an experiment to identify a hydroxylated psilocybin derivative compound having the chemical formula (XV) set forth herein (FIG. 18B).

FIGS. 19A and 19B depict a representation of mass spectrometry data in the form of a chromatogram, notably a chromatogram obtained in the performance of an experiment to synthesize an example hydroxylated psilocybin derivative compound having the chemical formula (XVI) set forth herein (FIG. 19A), and in the form of a mass spectrometry spectrum obtained in the performance of an experiment to identify a hydroxylated psilocybin derivative compound having the chemical formula (XVI) set forth herein (FIG. 19B).

FIGS. 20A and 20B depict a representation of mass spectrometry data in the form of a chromatogram, notably a chromatogram obtained in the performance of an experiment to synthesize an example hydroxylated psilocybin derivative compound having the chemical formula (XVIII) set forth herein (FIG. 20A), and in the form of a mass spectrometry spectrum obtained in the performance of an experiment to identify a hydroxylated psilocybin derivative compound having the chemical formula (XVIII) set forth herein (FIG. 20B).

FIGS. 21A and 21B depict a representation of mass spectrometry data in the form of a chromatogram, notably a chromatogram obtained in the performance of an experiment to synthesize an example hydroxylated psilocybin derivative compound having the chemical formula (XXVI) set forth herein (FIG. 21A), and in the form of a mass spectrometry spectrum obtained in the performance of an experiment to identify a hydroxylated psilocybin derivative compound having the chemical formula (XXVI) set forth herein (FIG. 21B).

FIGS. 22A and 22B depict a representation of mass spectrometry data in the form of a chromatogram, notably a chromatogram obtained in the performance of an experiment to synthesize an example hydroxylated psilocybin derivative compound having the chemical formula (XXVII) set forth herein (FIG. 22A), and in the form of a mass spectrometry spectrum obtained in the performance of an experiment to identify a hydroxylated psilocybin derivative compound having the chemical formula (XXVII) set forth herein (FIG. 22B).

FIGS. 23A and 23B depict a representation of mass spectrometry data in the form of a chromatogram, notably a chromatogram obtained in the performance of an experiment to synthesize an example hydroxylated psilocybin derivative compound having the chemical formula (XXIX) set forth herein (FIG. 23A), and in the form of a mass spectrometry spectrum obtained in the performance of an experiment to identify a hydroxylated psilocybin derivative compound having the chemical formula (XXIX) set forth herein (FIG. 23B).

The figures together with the following detailed description make apparent to those skilled in the art how the disclosure may be implemented in practice.

### DETAILED DESCRIPTION

Various compositions, systems or processes will be described below to provide an example of an embodiment of each claimed subject matter. No embodiment described below limits any claimed subject matter and any claimed subject matter may cover processes, compositions or systems that differ from those described below. The claimed subject matter is not limited to compositions, processes or systems having all of the features of any one composition, system or process described below or to features common to multiple or all of the compositions, systems or processes described below. It is possible that a composition, system or process described below is not an embodiment of any claimed subject matter. Any subject matter disclosed in a composition, system or process described below that is not claimed in this document may be the subject matter of another protective instrument, for example, a continuing patent application, and the applicant(s), inventor(s) or owner(s) do not intend to abandon, disclaim or dedicate to the public any such subject matter by its disclosure in this document.

As used herein and in the claims, the singular forms, such “a”, “an” and “the” include the plural reference and vice versa unless the context clearly indicates otherwise. Throughout this specification, unless otherwise indicated, “comprise,” “comprises” and “comprising” are used inclusively rather than exclusively, so that a stated integer or group of integers may include one or more other non-stated integers or groups of integers.

Various compositions, systems or processes will be described below to provide an example of an embodiment of each claimed subject matter. No embodiment described below limits any claimed subject matter and any claimed subject matter may cover processes, compositions or systems that differ from those described below. The claimed subject matter is not limited to compositions, processes or systems having all of the features of any one composition, system or process described below or to features common to multiple or all of the compositions, systems or processes described below. It is possible that a composition, system or process described below is not an embodiment of any claimed subject matter. Any subject matter disclosed in a composition, system or process described below that is not claimed in this document may be the subject matter of another protective instrument, for example, a continuing patent application, and the applicant(s), inventor(s) or owner(s) do not intend to abandon, disclaim or dedicate to the public any such subject matter by its disclosure in this document.

When ranges are used herein for physical properties, such as molecular weight, or chemical properties, such as chemical formulae, all combinations and sub-combinations of ranges and specific embodiments therein are intended to be included. Other than in the operating examples, or where otherwise indicated, all numbers expressing quantities of ingredients or reaction conditions used herein should be understood as modified in all instances by the term “about.” The term “about” when referring to a number or a numerical range means that the number or numerical range referred to is an approximation within experimental variability (or within statistical experimental error), and thus the number or numerical range may vary between 1% and 15% of the stated number or numerical range, as will be readily recognized by context. Furthermore any range of values described herein is intended to specifically include the limiting values of the range, and any intermediate value or sub-range within the given range, and all such intermediate values and sub-ranges are individually and specifically disclosed (e.g. a range of 1 to 5 includes 1, 1.5, 2, 2.75, 3, 3.90, 4, and 5). Similarly, other terms of degree such as “substantially” and “approximately” as used herein mean a reasonable amount of deviation of the modified term such that the end result is not significantly changed. These terms of degree should be construed as including a deviation of the modified term if this deviation would not negate the meaning of the term it modifies.

Unless otherwise defined, scientific and technical terms used in connection with the formulations described herein shall have the meanings that are commonly understood by those of ordinary skill in the art. The terminology used herein is for the purpose of describing particular embodiments only, and is not intended to limit the scope of the present invention, which is defined solely by the claims.

All publications, patents and patent applications are herein incorporated by reference in their entirety to the same extent as if each individual publication, patent or patent application was specifically and individually indicated to be incorporated by reference in its entirety.

### Terms and Definitions

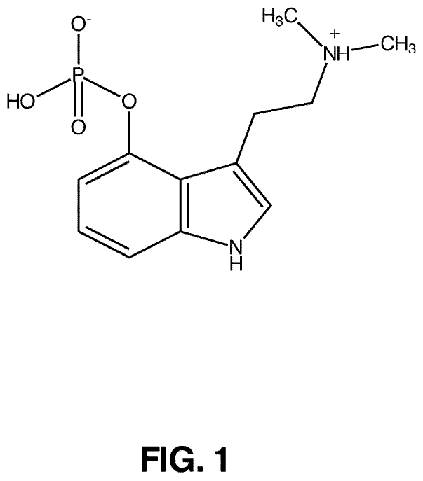

The term “psilocybin”, refers to a chemical compound having the structure set forth in FIG. 1.

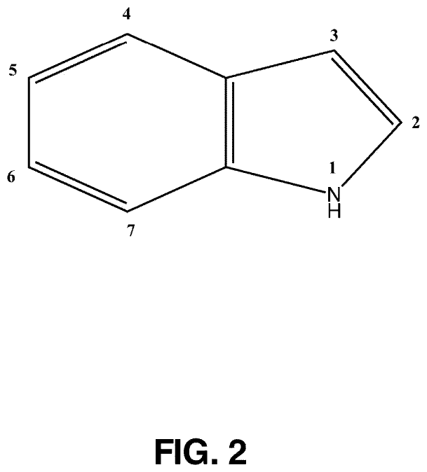

The term “indole prototype structure” refers to the chemical structure shown in FIG. 2. It is noted that specific carbon atoms and a nitrogen atom in the indole prototype structure are numbered. Reference may be made to these carbon and nitrogen numbers herein, for example C₂, C₄, N₁, and so forth. Furthermore, reference may be made to chemical groups attached to the indole prototype structure in accordance with the same numbering, for example R₄ and R₆ reference chemical groups attached to the C₄ and C₆ atom, respectively. In addition, R_{3A} and R_{3B}, in this respect, reference chemical groups extending from the 2-aminoethyl group extending in turn from the C₃ atom of the prototype indole structure.

The terms “hydroxy-containing psilocybin derivative” or hydroxy-containing psilocybin derivative compound” refer to a psilocybin derivative compound comprising one or more hydroxy groups. Reference may be made to specific carbon atoms which may be hydroxylated. For example, a 7-hydroxy-psilocybin derivative refers to a hydroxylated psilocybin derivative in which carbon atom number 7 (as identified in the indole prototype structure) is hydroxylated, or, similarly, 2-hydroxy-psilocybin derivative refers to a hydroxylated psilocybin derivative in which carbon atom number 2 (as identified in the indole prototype structure) is hydroxylated. Thus, for example, hydroxy-containing psilocybin derivatives include, single hydroxy derivatives, 2-hydroxy, 4-hydroxy, 5-hydroxy, 6-hydroxy and 7-hydroxy psilocybin derivatives, for example, and multiple hydroxy derivatives, such as, for example, 4,7-dihydroxy-psilocybin derivatives, and 2,5,7-tri-hydroxy-psilocybin derivatives. The term hydroxy-containing psilocybin derivatives further includes chemical compounds having the chemical formula (I):

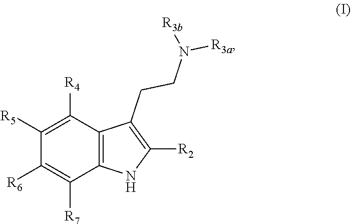

wherein at least one of R₂, R₄, R₅, R₆, or R₇ is a hydroxy group, and wherein each non-hydroxylated R₂, R₅, R₆, or R₇ is a hydrogen atom or an alkyl group or O-alkyl group, wherein R₄ when it is not hydroxylated is a hydrogen atom, an alkyl group or a phosphate group, and wherein R_{3A} and R_{3B} are a hydrogen, an alkyl group, an aryl group, or an acyl group. The term further includes salts of hydroxy-containing psilocybin derivatives, such as a sodium salt, a potassium salt etc.

The term “psilocybin precursor compound”, as used herein, refers to hydroxy-derivative psilocybin precursor compounds (as herein defined) and non-hydroxy-derivative psilocybin precursor compounds (as herein defined), together.

The term “hydroxy-derivative psilocybin precursor compound” refers to psilocybin precursor compounds possessing or derivatized to possess at least one hydroxy group and includes compounds selected from a hydroxylated tryptophan, a hydroxylated tryptamine and a hydroxylated indole, and further includes alkyl or O-alkyl derivatives thereof (e.g. C₂, C₄, C₅, C₆, or C₇ methyl, ethyl, propyl, O-methyl, O-ethyl, O-propyl derivatives). It is further noted that reference may be made to specific carbon atoms of the hydroxy-derivative psilocybin precursor compound which may be hydroxylated, for example, 6-hydroxy-tryptophan refers to a hydroxylated tryptophan in which carbon atom number 6 (as identified in the indole prototype structure) is hydroxylated, or, similarly, 2,4-di-hydroxytryptamine refers to a hydroxytryptamine in which carbon atom numbers 2 and 4 (as identified in the indole prototype structure) are hydroxylated. Further hydroxy-derivative psilocybin precursor compounds include 4-hydroxy-indole, 4-hydroxy-tryptophan, 4-hydroxy-tryptamine, 4-hydroxy-5-methyl-indole, and 4-hydroxy-7-methyl-indole.

The term “non-hydroxy-derivative psilocybin precursor compound” refers to any one of the psilocybin precursor compounds selected from tryptophan, tryptamine, and indole, and alkyl or O-alkyl derivatives thereof (e.g. C₂, C₄, C₅, C₆, or C₇ methyl, ethyl, propyl, O-methyl, O-ethyl, O-propyl derivatives). It is noted that non-hydroxy-derivative psilocybin precursor compound do not possess a hydroxy group. Further non-hydroxy-derivative psilocybin precursor compounds include 7-ethyl-indole, 7-ethyl-tryptophan, 5-methyl-indole.

The term “phosphate group”, as used herein, is a molecule containing one atom of phosphorus, covalently bound to four oxygen atoms (three single bonds and one double bond). Of the four oxygen atoms one oxygen atom may be a hydroxy group, and one of the non-hydroxylated oxygen atom may be chemically bonded to another entity.

The terms “hydroxy group”, and “hydroxy”, as used herein refers to a molecule containing one atom of oxygen bonded to one atom of hydrogen, and having the formula-OH. A hydroxy group through its oxygen atom may be chemically bonded to another entity.

The term “alkyl” as used herein refers to a straight and/or branched chain, saturated alkyl radical containing from one to “p” carbon atoms (“C₁-Cₚ-alkyl”) and includes, depending on the identity of “p”, methyl, ethyl, propyl, isopropyl, n-butyl, s-butyl, isobutyl, t-butyl, 2,2-dimethylbutyl, n-pentyl, 2-methylpentyl, 3-methylpentyl, 4-methylpentyl, n-hexyl and the like, where the variable p is an integer representing the largest number of carbon atoms in the alkyl radical. Alkyl groups further include hydrocarbon groups arranged in a chain having the chemical formula —CₙH₂ₙ₊₁, including, without limitation, methyl groups (—CH₃), ethyl groups (—C₂H₅), propyl groups (—C₃H₇), and butyl groups (—C₄H₉).

The term “alkylene” as used herein, whether alone or as part of another group, means an alkyl group (as defined herein) that is bivalent; i.e. that is substituted on two ends with another group. The term C₀₋₂alkylene means an alkylene group having 0, 1 or 2 carbon atoms.

The term “cycloalkyl” as used herein refers to a cyclic alkyl radical containing from three to “r” carbon atoms (“C₁-Cᵣ-cycloalkyl”) and includes, depending on the identity of “r”, cyclopropyl, cyclobutyl, cyclopentyl, cyclohexyl, cycloheptyl, and the like, where the variable r is an integer representing the largest number of carbon atoms in the cycloalkyl radical.

The term “O-alkyl”, as used herein, refers to a hydrocarbon group arranged in a chain having the chemical formula —O—CₙH₂ₙ₊₁. O-alkyl groups include, without limitation, O-methyl groups (—O—CH₃), O-ethyl groups (—O—C₂H₅), O-propyl groups (—O—C₃H₇) and O-butyl groups (—O—C₄H₉).

The term “aryl” as used herein refers to a monocyclic, bicyclic or tricyclic aromatic ring system containing, depending on the number of atoms in the rings, for example, from 6 to 14 carbon atoms (C₆-C₁₄-aryl) or from 6 to 10 carbons (C₆-C₁₀-aryl), and at least 1 aromatic ring and includes phenyl, naphthyl, anthracenyl, 1,2-dihydronaphthyl, 1,2,3,4-tetrahydronaphthyl, fluorenyl, phenanthrenyl, biphenylenyl, indanyl, indenyl and the like.

The term “alkaryl” as used herein refers to an aryl group, as defined herein, attached to the parent molecular group through an alkylene group as defined herein. In Some embodiments, the alkylene and the aryl each can be further substituted with 1, 2, 3, or 4 substituent groups as defined herein for the respective groups.

The term “acyl” refers to a carbon atom double bonded to an oxygen and single bonded to an alkyl group. The carbon atom further can be bonded to another entity. An acyl group can be described by the chemical formula: —C(═O)—CₙH₂ₙ₊₁.

The term “5-HT_{2A} receptor”, as used herein, refers to a subclass of a family of receptors for the neurotransmitter and peripheral signal mediator serotonin. 5-HT_{2A} receptors can mediate a plurality of central and peripheral physiologic functions of serotonin. Central nervous system effects can include mediation of hallucinogenic effects of hallucinogenic compounds.

The term “modulating 5-HT_{2A} receptors”, as used herein, refers to the ability of a compound disclosed herein to alter the function of 5-HT_{2A} receptors. A 5-HT_{2A} receptor modulator may activate the activity of a 5-HT_{2A} receptor, may activate or inhibit the activity of a 5-HT_{2A} receptor depending on the concentration of the compound exposed to the 5-HT_{2A} receptor, or may inhibit the activity of a 5-HT_{2A} receptor. Such activation or inhibition may be contingent on the occurrence of a specific event, such as activation of a signal transduction pathway, and/or maybe manifest only in particular cell types. The term “modulating 5-HT_{2A} receptors” also refers to altering the function of a 5-HT_{2A} receptor by increasing or decreasing the probability that a complex forms between a 5-HT_{2A} receptor and a natural binding partner to form a multimer. A 5-HT_{2A} receptor modulator may increase the probability that such a complex forms between the 5-HT_{2A} receptor and the natural binding partner, may increase or decrease the probability that a complex forms between the 5-HT_{2A} receptor and the natural binding partner depending on the concentration of the compound exposed to the 5-HT_{2A} receptor, and or may decrease the probability that a complex forms between the 5-HT_{2A} receptor and the natural binding partner. Furthermore, the term includes allosteric modulation of the receptor 5-HT_{2A}, i.e. modulation of the 5-HT_{2A} receptor through interaction with the 5-HT_{2A} receptor that is topographically different than the orthosteric site recognized by the cell's endogenous agonist, such modulation further including positive allosteric modulation (PAM), negative allosteric modulation (NAM) and silent allosteric modulation (SAM).

The term “5-HT_{2A} receptor-mediated disorder”, as used herein, refers to a disorder that is characterized by abnormal 5-HT_{2A} receptor activity. A 5-HT_{2A} receptor-mediated disorder may be completely or partially mediated by modulating 5-HT_{2A} receptors. In particular, a 5-HT_{2A} receptor-mediated disorder is one in which modulation of 5-HT_{2A} receptors results in some effect on the underlying disorder e.g., administration of a 5-HT_{2A} receptor modulator results in some improvement in at least some of the subjects being treated.

The term “pharmaceutical formulation”, as used herein, refers to a preparation in a form which allows an active ingredient, including a psychoactive ingredient, contained therein to provide effective treatment, and which does not contain any other ingredients which cause excessive toxicity, an allergic response, irritation, or other adverse response commensurate with a reasonable risk/benefit ratio. The pharmaceutical formulation may contain other pharmaceutical ingredients such as excipients, carriers, diluents, or auxiliary agents.

The term “recreational drug formulation”, as used herein, refers to a preparation in a form which allows a psychoactive ingredient contained therein to be effective for administration as a recreational drug, and which does not contain any other ingredients which cause excessive toxicity, an allergic response, irritation, or other adverse response commensurate with a reasonable risk/benefit ratio. The recreational drug formulation may contain other ingredients such as excipients, carriers, diluents, or auxiliary agents.

The term “effective for administration as a recreational drug”, as used herein, refers to a preparation in a form which allows a subject to voluntarily induce a psychoactive effect for non-medical purposes upon administration, generally in the form of self-administration. The effect may include an altered state of consciousness, satisfaction, pleasure, euphoria, perceptual distortion, or hallucination.

The term “effective amount”, as used herein, refers to an amount of an active agent, pharmaceutical formulation or recreational drug formulation, sufficient to induce a desired biological or therapeutic effect, including a prophylactic effect, and further including a psychoactive effect. Such effect can include an effect with respect to the signs, symptoms or causes of a disorder, or disease or any other desired alteration of a biological system. The effective amount can vary depending, for example, on the health condition, injury stage, disorder stage, or disease stage, weight, or sex of a subject being treated, timing of the administration, manner of the administration, age of the subject, and the like, all of which can be determined by those of skill in the art.

The terms “treating” and “treatment”, and the like, as used herein, are intended to mean obtaining a desirable physiological, pharmacological, or biological effect, and includes prophylactic and therapeutic treatment. The effect may result in the inhibition, attenuation, amelioration, or reversal of a sign, symptom or cause of a disorder, or disease, attributable to the disorder, or disease, which includes mental and psychiatric diseases and disorders. Clinical evidence of the prevention or treatment may vary with the disorder, or disease, the subject and the selected treatment.

The term “pharmaceutically acceptable”, as used herein, refers to materials, including excipients, carriers, diluents, or auxiliary agents, that are compatible with other materials in a pharmaceutical or recreational drug formulation and within the scope of reasonable medical judgement suitable for use in contact with a subject without excessive toxicity, allergic response, irritation, or other adverse response commensurate with a reasonable risk/benefit ratio.

The term “psilocybin biosynthetic enzyme complement”, as used herein, refers to one or more polypeptides which alone or together are capable of facilitating the chemical conversion of a psilocybin precursor compound and form another psilocybin precursor compound or psilocybin or a hydroxylated form thereof. A psilocybin biosynthetic enzyme complement can include, for example, PsiD, PsiH, PsiK, PsiM, PsiP, Psi-ncAAAD and TrpB.

The term “PsiD” as used herein, refers to any and all enzymes comprising a sequence of amino acid residues which is (i) substantially identical to the amino acid sequences constituting any PsiD polypeptide set forth herein, including, for example, SEQ.ID NO: 2, SEQ.ID NO: 10 and SEQ.ID NO: 32, or (ii) encoded by a nucleic acid sequence capable of hybridizing under at least moderately stringent conditions to any nucleic acid sequence encoding any PsiD set forth herein, but for the use of synonymous codons.

The term “PsiH” as used herein, refers to any and all enzymes comprising a sequence of amino acid residues which is (i) substantially identical to the amino acid sequences constituting any PsiH polypeptide set forth herein, including, for example, SEQ.ID NO: 4, or (ii) encoded by a nucleic acid sequence capable of hybridizing under at least moderately stringent conditions to any nucleic acid sequence encoding any PsiH set forth herein, but for the use of synonymous codons.

The term “PsiK” as used herein, refers to any and all enzymes comprising a sequence of amino acid residues which is (i) substantially identical to the amino acid sequences constituting any PsiK polypeptide set forth herein, including, for example, SEQ.ID NO: 6, or (ii) encoded by a nucleic acid sequence capable of hybridizing under at least moderately stringent conditions to any nucleic acid sequence encoding any PsiK set forth herein, but for the use of synonymous codons.

The term “PsiM” as used herein, refers to any and all enzymes comprising a sequence of amino acid residues which is (i) substantially identical to the amino acid sequences constituting any PsiM polypeptide set forth herein, including, for example, SEQ.ID NO: 8, or (ii) encoded by a nucleic acid sequence capable of hybridizing under at least moderately stringent conditions to any nucleic acid sequence encoding any PsiM set forth herein, but for the use of synonymous codons.

The term “Psi-ncAAAD” as used herein, refers to any and all enzymes comprising a sequence of amino acid residues which is (i) substantially identical to the amino acid sequences constituting any Psi-ncAAAD polypeptide set forth herein, including, for example, SEQ. ID NO: 10, or (ii) encoded by a nucleic acid sequence capable of hybridizing under at least moderately stringent conditions to any nucleic acid sequence encoding any Psi-ncAAAD set forth herein, but for the use of synonymous codons.

The term “TrpB” as used herein, refers to any and all enzymes comprising a sequence of amino acid residues which is (i) substantially identical to the amino acid sequences constituting any TrpB polypeptide set forth herein, including, for example, SEQ.ID NO: 12 and SEQ.ID NO: 27, or (ii) encoded by a nucleic acid sequence capable of hybridizing under at least moderately stringent conditions to any nucleic acid sequence encoding any TrpB set forth herein, but for the use of synonymous codons.

The term “hydroxylase” as used herein, refers to any and all enzymes comprising a sequence of amino acid residues which is (i) substantially identical to the amino acid sequences constituting any hydroxylase polypeptide set forth herein, including, for example, SEQ.ID NO: 14, SEQ.ID NO: 16, SEQ.ID NO: 18, SEQ. ID NO: 20, SEQ.ID NO: 39 and SEQ. ID NO: 43, or (ii) encoded by a nucleic acid sequence capable of hybridizing under at least moderately stringent conditions to any nucleic acid sequence encoding any hydroxylase set forth herein, but for the use of synonymous codons.

The term “acetyl transferase” as used herein, refers to any and all enzymes comprising a sequence of amino acid residues which is (i) substantially identical to the amino acid sequences constituting any acetyl transferase polypeptide set forth herein, including, for example, SEQ. ID NO: 24, or (ii) encoded by a nucleic acid sequence capable of hybridizing under at least moderately stringent conditions to any nucleic acid sequence encoding any acetyl transferase set forth herein, but for the use of synonymous codons.

The terms “nucleic acid sequence encoding PsiD”, and “nucleic acid sequence encoding a PsiD polypeptide”, as may be used interchangeably herein, refer to any and all nucleic acid sequences encoding a PsiD polypeptide, including, for example, SEQ.ID NO: 1, SEQ.ID NO: 9 and SEQ.ID NO: 31. Nucleic acid sequences encoding a PsiD polypeptide further include any and all nucleic acid sequences which (i) encode polypeptides that are substantially identical to the PsiD polypeptide sequences set forth herein; or (ii) hybridize to any PsiD nucleic acid sequences set forth herein under at least moderately stringent hybridization conditions or which would hybridize thereto under at least moderately stringent conditions but for the use of synonymous codons.

The terms “nucleic acid sequence encoding PsiH”, and “nucleic acid sequence encoding a PsiH polypeptide”, as may be used interchangeably herein, refer to any and all nucleic acid sequences encoding a PsiH polypeptide, including, for example, SEQ.ID NO: 3. Nucleic acid sequences encoding a PsiH polypeptide further include any and all nucleic acid sequences which (i) encode polypeptides that are substantially identical to the PsiH polypeptide sequences set forth herein; or (ii) hybridize to any PsiH nucleic acid sequences set forth herein under at least moderately stringent hybridization conditions or which would hybridize thereto under at least moderately stringent conditions but for the use of synonymous codons.

The terms “nucleic acid sequence encoding PsiK”, and “nucleic acid sequence encoding a PsiK polypeptide”, as may be used interchangeably herein, refer to any and all nucleic acid sequences encoding a PsiK polypeptide, including, for example, SEQ. ID NO: 5. Nucleic acid sequences encoding a PsiK polypeptide further include any and all nucleic acid sequences which (i) encode polypeptides that are substantially identical to the PsiK polypeptide sequences set forth herein; or (ii) hybridize to any PsiK nucleic acid sequences set forth herein under at least moderately stringent hybridization conditions or which would hybridize thereto under at least moderately stringent conditions but for the use of synonymous codons.

The terms “nucleic acid sequence encoding PsiM”, and “nucleic acid sequence encoding a PsiM polypeptide”, as may be used interchangeably herein, refer to any and all nucleic acid sequences encoding a PsiD polypeptide, including, for example, SEQ.ID NO: 7. Nucleic acid sequences encoding a PsiM polypeptide further include any and all nucleic acid sequences which (i) encode polypeptides that are substantially identical to the PsiM polypeptide sequences set forth herein; or (ii) hybridize to any PsiM nucleic acid sequences set forth herein under at least moderately stringent hybridization conditions or which would hybridize thereto under at least moderately stringent conditions but for the use of synonymous codons.

The terms “nucleic acid sequence encoding Psi-ncAAAD”, and “nucleic acid sequence encoding a Psi-ncAAAD polypeptide”, as may be used interchangeably herein, refer to any and all nucleic acid sequences encoding a PsiD polypeptide, including, for example, SEQ.ID NO: 9. Nucleic acid sequences encoding a Psi-ncAAAD polypeptide further include any and all nucleic acid sequences which (i) encode polypeptides that are substantially identical to the Psi-ncAAAD polypeptide sequences set forth herein; or (ii) hybridize to any Psi-ncAAAD nucleic acid sequences set forth herein under at least moderately stringent hybridization conditions or which would hybridize thereto under at least moderately stringent conditions but for the use of synonymous codons.

The terms “nucleic acid sequence encoding TrpB”, and “nucleic acid sequence encoding a TrpB polypeptide”, as may be used interchangeably herein, refer to any and all nucleic acid sequences encoding a TrpB polypeptide, including, for example, SEQ.ID NO: 11 and SEQ.ID NO: 26. Nucleic acid sequences encoding a TrpB polypeptide further include any and all nucleic acid sequences which (i) encode polypeptides that are substantially identical to the TrpB polypeptide sequences set forth herein; or (ii) hybridize to any TrpB nucleic acid sequences set forth herein under at least moderately stringent hybridization conditions or which would hybridize thereto under at least moderately stringent conditions but for the use of synonymous codons.

The terms “nucleic acid sequence encoding a hydroxylase”, and “nucleic acid sequence encoding a hydroxylase polypeptide”, as may be used interchangeably herein, refer to any and all nucleic acid sequences encoding a hydroxylase polypeptide, including, for example, SEQ.ID NO: 13, SEQ.ID NO: 15, SEQ.ID NO: 17, SEQ. ID NO: 19, SEQ.ID NO: 38 and SEQ. ID NO: 42. Nucleic acid sequences encoding a hydroxylase polypeptide further include any and all nucleic acid sequences which (i) encode polypeptides that are substantially identical to the hydroxylase polypeptide sequences set forth herein; or (ii) hybridize to any hydroxylase nucleic acid sequences set forth herein under at least moderately stringent hybridization conditions or which would hybridize thereto under at least moderately stringent conditions but for the use of synonymous codons.

The terms “nucleic acid sequence encoding acetyl transferase”, and “nucleic acid sequence encoding an acetyl. transferase polypeptide”, as may be used interchangeably herein, refer to any and all nucleic acid sequences encoding an acetyl transferase polypeptide, including, for example, SEQ.ID NO: 25. Nucleic acid sequences encoding an acetyl transferase polypeptide further include any and all nucleic acid sequences which (i) encode polypeptides that are substantially identical to the acetyl transferase polypeptide sequences set forth herein; or (ii) hybridize to any acetyl transferase nucleic acid sequences set forth herein under at least moderately stringent hybridization conditions or which would hybridize thereto under at least moderately stringent conditions but for the use of synonymous codons.

The terms “nucleic acid”, or “nucleic acid sequence”, as used herein, refer to a sequence of nucleoside or nucleotide monomers, consisting of naturally occurring bases, sugars and intersugar (backbone) linkages. The term also includes modified or substituted sequences comprising non-naturally occurring monomers or portions thereof. The nucleic acids of the present disclosure may be deoxyribonucleic nucleic acids (DNA) or ribonucleic acids (RNA) and may include naturally occurring bases including adenine, guanine, cytosine, thymidine and uracil. The nucleic acids may also contain modified bases. Examples of such modified bases include aza and deaza adenine, guanine, cytosine, thymidine and uracil, and xanthine and hypoxanthine. A sequence of nucleotide or nucleoside monomers may be referred to as a polynucleotide sequence, nucleic acid sequence, a nucleotide sequence or a nucleoside sequence.

The term “polypeptide”, as used herein in conjunction with a reference SEQ.ID NO, refers to any and all polypeptides comprising a sequence of amino acid residues which is (i) substantially identical to the amino acid sequence constituting the polypeptide having such reference SEQ.ID NO, or (ii) encoded by a nucleic acid sequence capable of hybridizing under at least moderately stringent conditions to any nucleic acid sequence encoding the polypeptide having such reference SEQ.ID NO, but for the use of synonymous codons. A sequence of amino acid residues may be referred to as an amino acid sequence, or polypeptide sequence.

The term “nucleic acid sequence encoding a polypeptide”, as used herein in conjunction with a reference SEQ.ID NO, refers to any and all nucleic acid sequences encoding a polypeptide having such reference SEQ.ID NO. Nucleic acid sequences encoding a polypeptide, in conjunction with a reference SEQ.ID NO, further include any and all nucleic acid sequences which (i) encode polypeptides that are substantially identical to the polypeptide having such reference SEQ.ID NO; or (ii) hybridize to any nucleic acid sequences encoding polypeptides having such reference SEQ.ID NO under at least moderately stringent hybridization conditions or which would hybridize thereto under at least moderately stringent conditions but for the use of synonymous codons.

By the term “substantially identical” it is meant that two amino acid sequences preferably are at least 70% identical, and more preferably are at least 85% identical and most preferably at least 95% identical, for example 96%, 97%, 98% or 99% identical. In order to determine the percentage of identity between two amino acid sequences the amino acid sequences of such two sequences are aligned, using for example the alignment method of Needleman and Wunsch (J. Mol. Biol., 1970, 48:443), as revised by Smith and Waterman (Adv. Appl. Math., 1981, 2:482) so that the highest order match is obtained between the two sequences and the number of identical amino acids is determined between the two sequences. Methods to calculate the percentage identity between two amino acid sequences are generally art recognized and include, for example, those described by Carillo and Lipton (SIAM J. Applied Math., 1988, 48:1073) and those described in Computational Molecular Biology, Lesk, e.d. Oxford University Press, New York, 1988, Biocomputing: Informatics and Genomics Projects. Generally, computer programs will be employed for such calculations. Computer programs that may be used in this regard include, but are not limited to, GCG (Devereux et al., Nucleic Acids Res., 1984, 12:387) BLASTP, BLASTN and FASTA (Altschul et al., J. Mol. Biol., 1990:215:403). A particularly preferred method for determining the percentage identity between two polypeptides involves the Clustal W algorithm (Thompson, J D, Higgines, D G and Gibson T J, 1994, Nucleic Acid Res 22 (22): 4673-4680 together with the BLOSUM 62 scoring matrix (Henikoff S & Henikoff, J G, 1992, Proc. Natl. Acad. Sci. USA 89:10915-10919 using a gap opening penalty of 10 and a gap extension penalty of 0.1, so that the highest order match obtained between two sequences wherein at least 50% of the total length of one of the two sequences is involved in the alignment.

By “at least moderately stringent hybridization conditions” it is meant that conditions are selected which promote selective hybridization between two complementary nucleic acid molecules in solution. Hybridization may occur to all or a portion of a nucleic acid sequence molecule. The hybridizing portion is typically at least 15 (e.g. 20, 25, 30, 40 or 50) nucleotides in length. Those skilled in the art will recognize that the stability of a nucleic acid duplex, or hybrids, is determined by the Tm, which in sodium containing buffers is a function of the sodium ion concentration and temperature (Tm=81.5° C.-16.6 (Log 10 [Na+])+0.41(% (G+C)-600/I), or similar equation). Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tm, for example if nucleic acid molecules are sought that have a >95% identity, the final wash temperature will be reduced by about 5° C. Based on these considerations those skilled in the art will be able to readily select appropriate hybridization conditions. In preferred embodiments, stringent hybridization conditions are selected. By way of example the following conditions may be employed to achieve stringent hybridization: hybridization at 5× sodium chloride/sodium citrate (SSC)/5×Denhardt's solution/1.0% SDS at Tm (based on the above equation)−5° C., followed by a wash of 0.2×SSC/0.1% SDS at 60° C. Moderately stringent hybridization conditions include a washing step in 3×SSC at 42° C. It is understood however that equivalent stringencies may be achieved using alternative buffers, salts and temperatures. Additional guidance regarding hybridization conditions may be found in: Current Protocols in Molecular Biology, John Wiley & Sons, N.Y., 1989, 6.3.1. -6.3.6 and in: Sambrook et al., Molecular Cloning, a Laboratory Manual, Cold Spring Harbor Laboratory Press, 1989, Vol. 3.

The term “functional variant”, as used herein in reference to polynucleotides or polypeptides, refers to polynucleotides or polypeptides capable of performing the same function as a noted reference polynucleotide or polypeptide. Thus, for example, a functional variant of the polypeptide set forth in SEQ.ID NO: 2, refers to a polypeptide capable of performing the same function as the polypeptide set forth in SEQ.ID NO: 2. Functional variants include modified a polypeptide wherein, relative to a noted reference polypeptide, the modification includes a substitution, deletion or addition of one or more amino acids. In some embodiments, substitutions are those that result in a replacement of one amino acid with an amino acid having similar characteristics. Such substitutions include, without limitation (i) glutamic acid and aspartic acid; (i) alanine, serine, and threonine; (iii) isoleucine, leucine and valine, (iv) asparagine and glutamine, and (v) tryptophan, tyrosine and phenylalanine. Functional variants further include polypeptides having retained or exhibiting an enhanced psilocybin biosynthetic bioactivity.

The term “chimeric”, as used herein in the context of nucleic acids, refers to at least two linked nucleic acids which are not naturally linked. Chimeric nucleic acids include linked nucleic acids of different natural origins. For example, a nucleic acid constituting a microbial promoter linked to a nucleic acid encoding a plant polypeptide is considered chimeric. Chimeric nucleic acids also may comprise nucleic acids of the same natural origin, provided they are not naturally linked. For example a nucleic acid constituting a promoter obtained from a particular cell-type may be linked to a nucleic acid encoding a polypeptide obtained from that same cell-type, but not normally linked to the nucleic acid constituting the promoter. Chimeric nucleic acids also include nucleic acids comprising any naturally occurring nucleic acids linked to any non-naturally occurring nucleic acids.

The terms “substantially pure” and “isolated”, as may be used interchangeably herein describe a compound, e.g., a secondary metabolite, psilocybin or a psilocybin derivative, polynucleotide or a polypeptide, which has been separated from components that naturally accompany it. Typically, a compound is substantially pure when at least 60%, more preferably at least 75%, more preferably at least 90%, 95%, 96%, 97%, or 98%, and most preferably at least 99% of the total material (by volume, by wet or dry weight, or by mole percent or mole fraction) in a sample is the compound of interest. Purity can be measured by any appropriate method, e.g., in the case of polypeptides, by chromatography, gel electrophoresis or HPLC analysis.

The term “recovered” as used herein in association with an enzyme, protein, a secondary metabolite or a chemical compound, refers to a more or less pure form of the enzyme, protein, secondary metabolite, or chemical compound.

### General Implementation

As hereinbefore mentioned, the present disclosure relates to psilocybin derivatives. In particular, the present disclosure provides novel hydroxylated psilocybin derivatives. In general, the herein provided compositions exhibit functional properties which deviate from the functional properties of psilocybin. Thus, for example, the hydroxylated psilocybin derivatives, can exhibit pharmacological properties which deviate from psilocybin. Furthermore, the hydroxylated derivatives may psilocybin derivatives may exhibit physico-chemical properties which differ from psilocybin. Thus, for example, hydroxylated psilocybin derivatives may exhibit superior solubility in a solvent, for example, an aqueous solvent. The hydroxylated psilocybin derivatives in this respect are useful in the formulation of pharmaceutical and recreational drug formulations. Furthermore, the hydroxylated psilocybin compounds of the present disclosure may be used as a feedstock material for deriving further psilocybin derivatives. In one embodiment, the hydroxylated psilocybin derivatives of the present disclosure can conveniently be biosynthetically produced. The practice of this method avoids the extraction of psilocybin from mushrooms and the performance of subsequent chemical reactions to achieve hydroxylated derivatives. Furthermore, the growth of mushrooms can be avoided thus limiting the dependence on climate and weather, and potential legal and social challenges associated with the cultivation of mushrooms containing psychoactive compounds. The method can efficiently yield substantial quantities of hydroxylated psilocybin derivatives.

In what follows selected embodiments are described with reference to the drawings.

Initially example hydroxylated psilocybin derivatives will be described. Thereafter example methods of using and making the hydroxylated psilocybin derivatives will be described.

Accordingly, in one aspect the present disclosure provides derivatives of a compound known as psilocybin of which the chemical structure is shown in FIG. 1. The derivatives herein provided are, in particular, derivatives of psilocybin including a hydroxy group.

Thus, in one aspect, the present disclosure provides, in accordance with the teachings herein, in at least one embodiment, a chemical compound or salt thereof having formula (I):

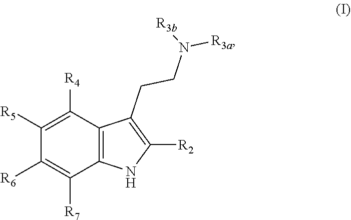

wherein at least one of R₂, R₄, R₅, R₆, or R₇ is a hydroxy group, and wherein each non-hydroxylated R₂, R₅, R₆, or R₇ is a hydrogen atom or an alkyl group or O-alkyl group, wherein R₄ when it is not hydroxylated is a hydrogen atom, an alkyl group or a phosphate group, and wherein R_{3A} and R_{3B} are a hydrogen, an alkyl group, a cycloalkyl group, an aryl group, an alkaryl group or an acyl group.

Thus, referring to the chemical compound having formula (I), initially it is noted that, in an aspect thereof, at least one of R₂, R₄, R₅, R₆, or R₇ is a hydroxy group.

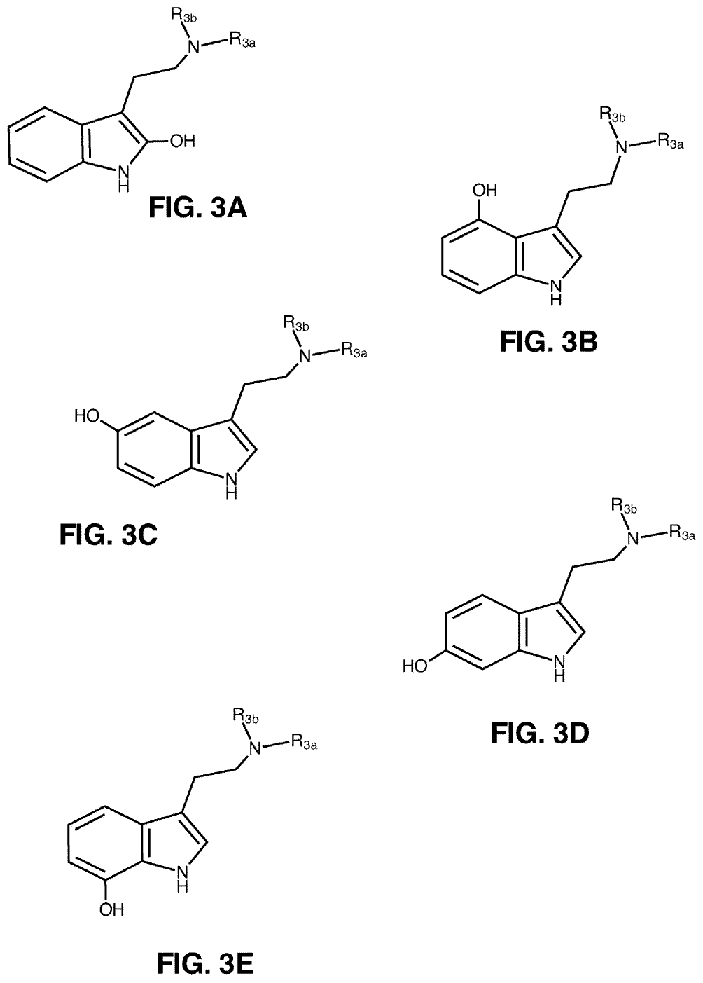

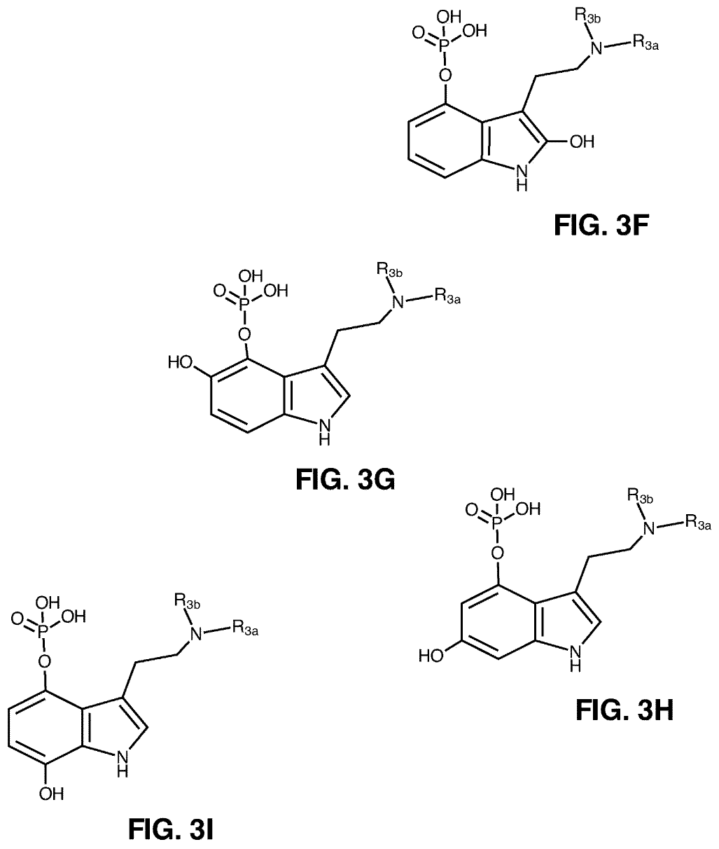

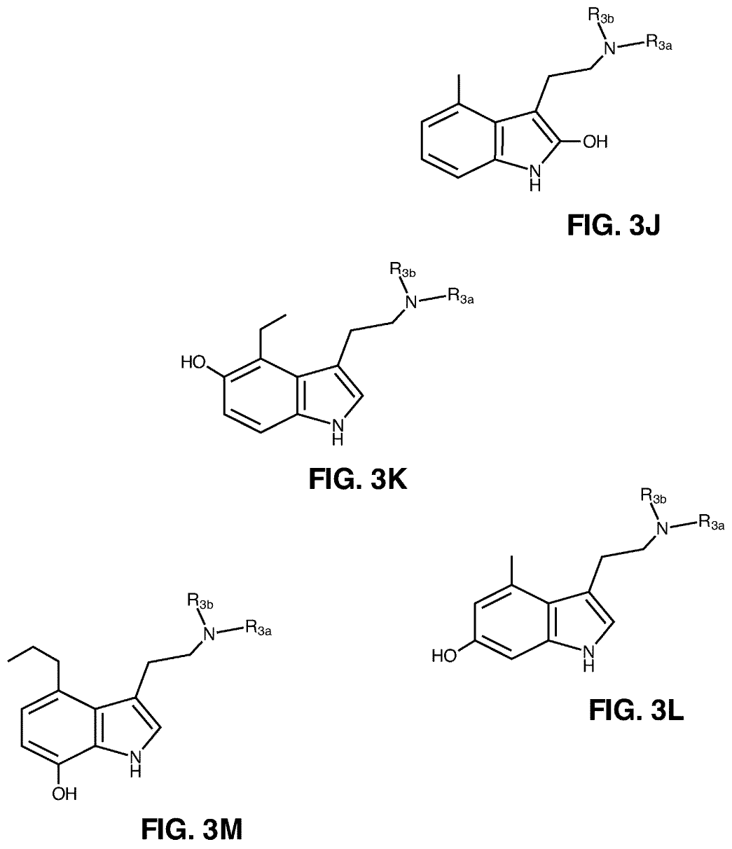

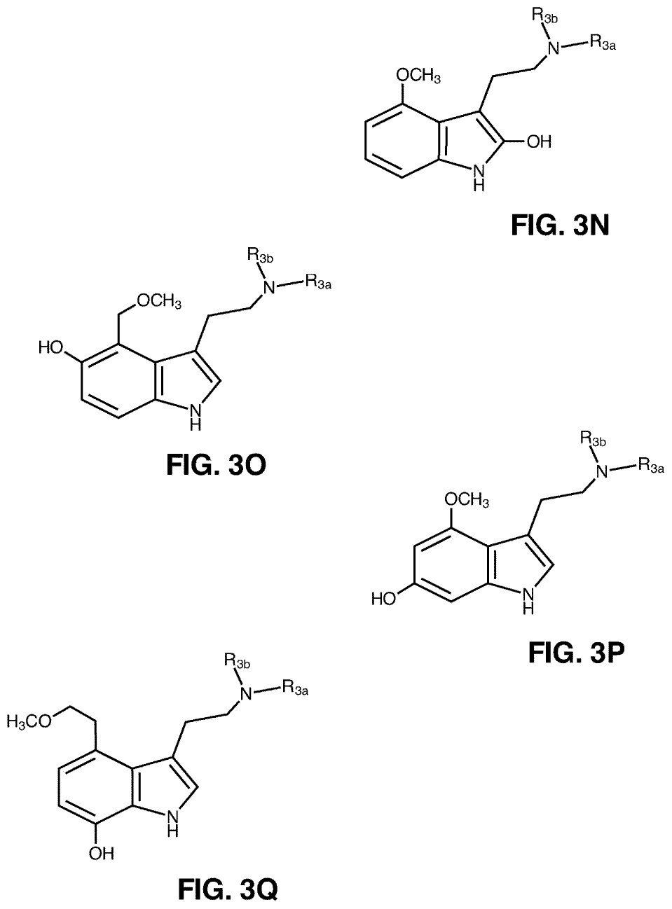

Continuing to refer to the chemical compound having formula (I), in one embodiment, one of R₂, R₄, R₅, R₆ and R₇ can be a hydroxy group. Thus, in one embodiment, R₂ can be a hydroxy group, each of R₅, R₆ and R₇ can be a hydrogen atom or an alkyl group or O-alkyl group, and R₄ can be a phosphate group, a hydrogen atom or an alkyl group or O-alkyl group (see: the example hydroxylated psilocybin derivatives shown in FIG. 3A (R₂ is a hydroxy group; R₄ is a hydrogen atom; R₅, R₆ and R₇ are a hydrogen atom; R₃ₐ and R_{3b} are a hydrogen atom, an alkyl group, a cycloalkyl group, an alkaryl group, an aryl group, or an acyl group); FIG. 3F (R₂ is a hydroxy group; R₄ is a phosphate group; R₅, R₆ and R₇ are a hydrogen atom; R₃ₐ and R_{3b} are a hydrogen atom, an alkyl group, a cycloalkyl group, an alkaryl group, an aryl group, or an acyl group); FIG. 3J (R₂ is a hydroxy group; R₄ is a methyl group; R₅, R₆ and R₇ are a hydrogen atom; R₃ₐ and R_{3b} are a hydrogen atom, an alkyl group, a cycloalkyl group, an alkaryl group, an aryl group, or an acyl group); and FIG. 3N (R₂ is a hydroxy group; R₄ is an O-methyl group; R₅, R₆ and R₇ are a hydrogen atom; R₃ₐ and R_{3b} are a hydrogen atom, an alkyl group, a cycloalkyl group, an alkaryl group, an aryl group, or an acyl group)).

Continuing to refer to the chemical compound having formula (I), in one embodiment, R₄ can be a hydroxy group, and each of R₂, R₅, R₆ and R₇ can be a hydrogen atom or an alkyl group or O-alkyl group (see: the example hydroxylated psilocybin derivative shown in FIG. 3B (R₄ is a hydroxy group; R₂, R₅, R₆ and R₇ are a hydrogen atom; R₃ₐ and R_{3b} are a hydrogen atom, an alkyl group, a cycloalkyl group, an alkaryl group, an aryl group, or an acyl group)).

Continuing to refer to the chemical compound having formula (I), in one embodiment, R₅ can be a hydroxy group, and each of R₂, R₆ and R₇ can be a hydrogen atom or an alkyl group or O-alkyl group, and R₄ can be a phosphate group, a hydrogen atom or a hydroxy group (see: the example hydroxylated psilocybin derivatives shown in FIG. 3C (R₅ is a hydroxy group; R₄ is a hydrogen atom; R₂, R₆ and R₇ are a hydrogen atom; R₃ₐ and R_{3b} are a hydrogen atom, an alkyl group, a cycloalkyl group, an alkaryl group, an aryl group, or an acyl group); FIG. 3G (R₅ is a hydroxy group; R₄ is a phosphate group; R₄, R₆ and R₇ are a hydrogen atom; R₃ₐ and R_{3b} are a hydrogen atom, an alkyl group, a cycloalkyl group, an alkaryl group, an aryl group, or an acyl group); FIG. 3K (R₅ is a hydroxy group; R₄ is a ethyl group; R₄, R₆ and R₇ are a hydrogen atom; R₃ₐ and R_{3b} are a hydrogen atom, an alkyl group, a cycloalkyl group, an alkaryl group, an aryl group, or an acyl group); and FIG. 3O (R₅ is a hydroxy group; R₄ is an O-ethyl group; R₄, R₆ and R₇ are a hydrogen atom; R₃ₐ and R_{3b} are a hydrogen atom, an alkyl group, a cycloalkyl group, an alkaryl group, an aryl group, or an acyl group).

Continuing to refer to the chemical compound having formula (I), in one embodiment, R₆ can be a hydroxy group, and each of R₂, R₅ and R₇ can be a hydrogen atom or an alkyl group or O-alkyl group, and R₄ can be a phosphate group, a hydrogen atom or an alkyl group or O-alkyl group (see: the example hydroxylated psilocybin derivatives shown in FIG. 3D (R₆ is a hydroxy group; R₄ is a phosphate group; R₂, R₅ and R₇ are a hydrogen atom; R₃ₐ and R_{3b} are a hydrogen atom, an alkyl group, a cycloalkyl group, an alkaryl group, an aryl group, or an acyl group) and FIG. 3H (R₆ is a hydroxy group; R₄ is a phosphate group; R₂, R₅ and R₇ are a hydrogen atom; R₃ₐ and R_{3b} are a hydrogen atom, an alkyl group, a cycloalkyl group, an alkaryl group, an aryl group, or an acyl group); FIG. 3L (R₆ is a hydroxy group; R₄ is a methyl group; R₂, R₅ and R₇ are a hydrogen atom; R₃ₐ and R_{3b} are a hydrogen atom, an alkyl group, a cycloalkyl group, an alkaryl group, an aryl group, or an acyl group); and FIG. 3P (R₆ is a hydroxy group; R₄ is an O-methyl group; R₂, R₅ and R₇ are a hydrogen atom; R₃ₐ and R_{3b} are a hydrogen atom, an alkyl group, a cycloalkyl group, an alkaryl group, an aryl group, or an acyl group)).

Continuing to refer to the chemical compound having formula (I), in one embodiment, R₇ can be a hydroxy group, and each of R₂, R₅ and R₆ can be a hydrogen atom or an alkyl group or O-alkyl group, and R₄ can be a phosphate group, a hydrogen atom or an alkyl group or O-alkyl group (see: the example hydroxylated psilocybin derivatives shown in FIG. 3E (R₇ is a hydroxy group; R₄ is a hydrogen atom; R₂, R₅ and R₆ are a hydrogen atom R₃ₐ and R_{3b} are a hydrogen atom, an alkyl group, a cycloalkyl group, an alkaryl group, an aryl group, or an acyl group); FIG. 3I (R₇ is a hydroxy group; R₄ is a phosphate group; R₂, R₅ and R₆ are a hydrogen atom; R₃ₐ and R_{3b} are a hydrogen atom, an alkyl group, a cycloalkyl group, an alkaryl group, an aryl group, or an acyl group); FIG. 3M (R₇ is a hydroxy group; R₄ is a propyl group; R₂, R₅ and R₆ are a hydrogen atom; R₃ₐ and R_{3b} are a hydrogen atom, an alkyl group, a cycloalkyl group, an alkaryl group, an aryl group, or an acyl group); and FIG. 3Q (R₇ is a hydroxy group; R₄ is an O-propyl group; R₂, R₅ and R₆ are a hydrogen atom; R₃ₐ and R_{3b} are a hydrogen atom, an alkyl group, a cycloalkyl group, an alkaryl group, an aryl group, or an acyl group)).

In some embodiments, two of R₂, R₄, R₅, R₆ and R₇ of the chemical compound having formula (I) can be hydroxy groups. Thus, continuing to refer to the chemical compound having formula (I), in one embodiment, two of R₂, R₄, R₅, R₆ and R₇ can be a hydroxy group, wherein each non-hydroxylated R₂, R₅, R₆ and R₇ is a hydrogen atom or an alkyl group or O-alkyl group, and wherein R₄, when it is not a hydroxy group, is a phosphate group, a hydrogen atom or an alkyl group or O-alkyl group.

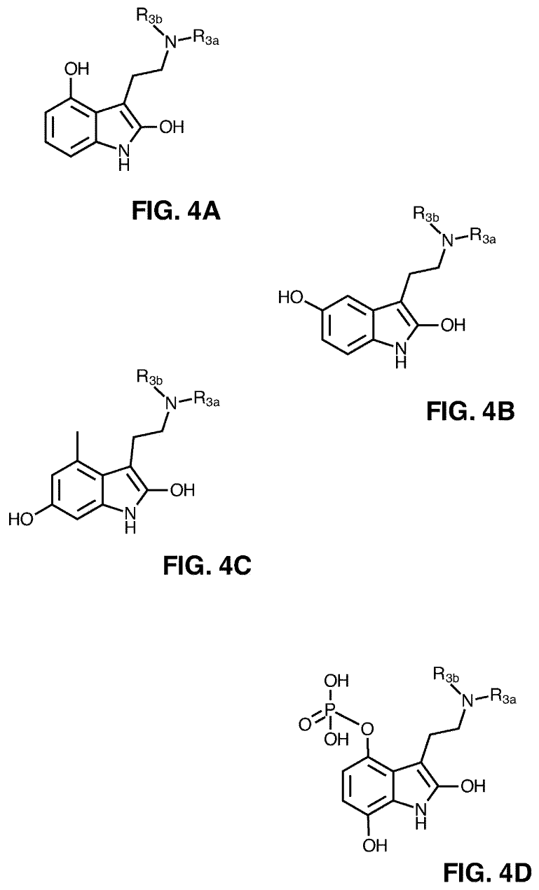

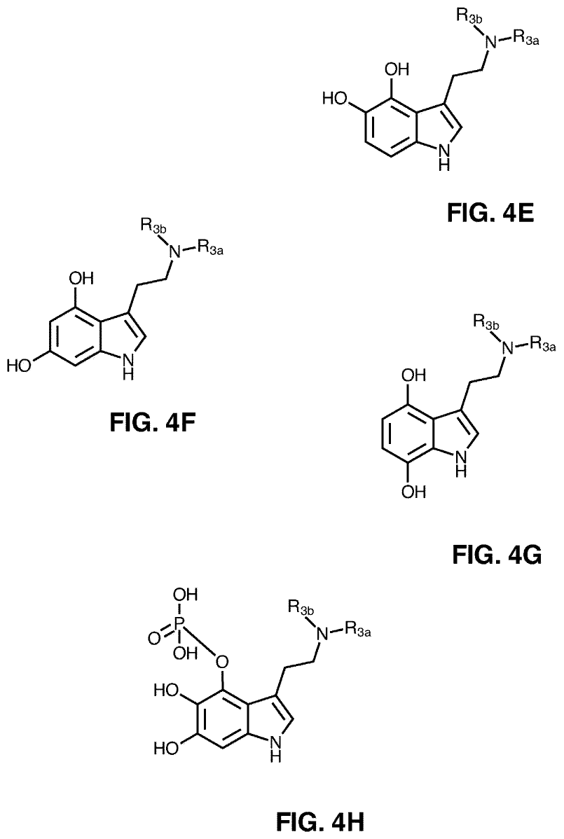

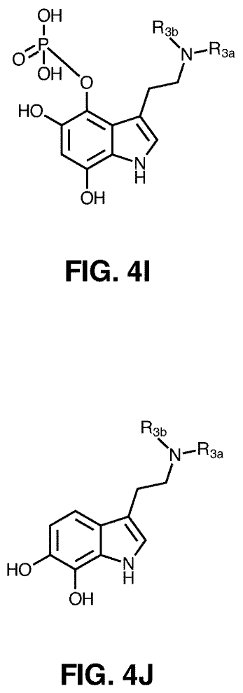

Still continuing to refer to the chemical compound having formula (I), in one embodiment, R₂ and R₄ can be hydroxy groups and R₅, R₆ and R₇ can be hydrogen atoms or alkyl groups (see: the example hydroxylated psilocybin derivative shown in FIG. 4A (R₂ and R₄ are each a hydroxy group; R₅, R₆ and R₇ are hydrogen atoms; and R₃ₐ and R_{3b} are a hydrogen atom, an alkyl group, a cycloalkyl group, an alkaryl group, an aryl group, or an acyl group)).

Continuing to refer to the chemical compound having formula (I), in one embodiment, R₂ and R₅ can be hydroxy groups, and R₆ and R₇ can be hydrogen atoms or alkyl groups and R₄ can be a phosphate group, a hydrogen atom or an alkyl group or O-alkyl group (see: the example hydroxylated psilocybin derivative shown in FIG. 4B (R₂ and R₅ are each a hydroxy group; R₄, R₆ and R₇ are hydrogen atoms; and R₃ₐ and R_{3b} are a hydrogen atom, an alkyl group, a cycloalkyl group, an alkaryl group, an aryl group, or an acyl group)).

Continuing to refer to the chemical compound having formula (I), in one embodiment, R₂ and R₆ can hydroxy groups, and R₅ and R₇ can be hydrogen atoms, and R₄ can be a phosphate group, a hydrogen atom or an alkyl group or O-alkyl group (see: the example hydroxylated psilocybin derivative shown in FIG. 4C (R₂ and R₆ are each a hydroxy group; R₄ is a methyl group, R₅ and R₇ are hydrogen atoms; and R₃ₐ and R_{3b} are a hydrogen atom, an alkyl group, a cycloalkyl group, an alkaryl group, an aryl group, or an acyl group)).

Continuing to refer to the chemical compound having formula (I), in one embodiment, R₂ and R₇ can be hydroxy groups, and R₅ and R₆ can be hydrogen atoms or alkyl group and R₄ can be a phosphate group, a hydrogen atom or an alkyl group or O-alkyl group (see: the example hydroxylated psilocybin derivative shown in FIG. 4D (R₂ and R₇ are each hydroxy groups; R₄ is a phosphate group; R₅ and R₆ are hydrogen atoms; and R₃ₐ and R_{3b} are a hydrogen atom, an alkyl group, a cycloalkyl group, an alkaryl group, an aryl group, or an acyl group)).

In one embodiment, R₄ and R₅ can be hydroxy groups, R₂, R₆ and R₇ can be hydrogen atoms or alkyl groups (see: the example hydroxylated psilocybin derivative shown in FIG. 4E (R₄ and R₅ are each hydroxy groups; R₂, R₆ and R₇ are hydrogen atoms; and R₃ₐ and R_{3b} are a hydrogen atom, an alkyl group, a cycloalkyl group, an alkaryl group, an aryl group, or an acyl group)).

Continuing to refer to the chemical compound having formula (I), in one embodiment, R₄ and R₆ can be hydroxy groups, and R₂, R₅ and R₇ can be hydrogen atoms or alkyl groups (see: the example hydroxylated psilocybin derivative shown in FIG. 4F (R₄ and R₆ are each hydroxy groups; R₂, R₅ and R₇ are hydrogen atoms; and R₃ₐ and R_{3b} are a hydrogen atom, an alkyl group, a cycloalkyl group, an alkaryl group, an aryl group, or an acyl group)).

Continuing to refer to the chemical compound having formula (I), in one embodiment, R₄ and R₇ can be hydroxy groups, and R₂, R₅ and R₆ can be hydrogen atoms or alkyl groups (see: the example hydroxy psilocybin derivative shown in FIG. 4G (R₄ and R₇ are each hydroxy groups; R₂, R₅ and R₆ are hydrogen atoms; and R₃ₐ and R_{3b} are a hydrogen atom, an alkyl group, a cycloalkyl group, an alkaryl group, an aryl group, or an acyl group)).

Continuing to refer to the chemical compound having formula (I), in one embodiment, R₅ and R₆ can be hydroxy groups and R₂ and R₇ can be hydrogen atoms or alkyl groups and R₄ can be a phosphate group, a hydrogen atom or an alkyl group or O-alkyl group (see: the example hydroxylated psilocybin derivative shown in FIG. 4H (R₅ and R₆ are each hydroxy groups; R₄ is a phosphate group; R₂ and R₇ are hydrogen atoms; and R₃ₐ and R_{3b} are a hydrogen atom, an alkyl group, a cycloalkyl group, an alkaryl group, an aryl group, or an acyl group)).

Continuing to refer to the chemical compound having formula (I), in one embodiment, R₅ and R₇ can be hydroxy groups, and R₂ and R₆ can be hydrogen atoms or alkyl groups and R₄ can be a phosphate group, a hydrogen atom or an alkyl group or O-alkyl group (see: the example hydroxylated psilocybin derivative shown in FIGS. 4I (R₅ and R₇ are each hydroxy groups; R₄ is a phosphate group; R₂ and R₆ are hydrogen atoms; and R₃ₐ and R_{3b} are a hydrogen atom, an alkyl group, a cycloalkyl group, an alkaryl group, an aryl group, or an acyl group)).

Continuing to refer to the chemical compound having formula (I), in one embodiment, R₆ and R₇ can be hydroxy groups, and R₂ and R₅ can be hydrogen atoms or alkyl groups and R₄ can be a phosphate group, a hydrogen atom or an alkyl group or O-alkyl group (see: the example hydroxylated psilocybin derivative shown in FIG. 4J (R₆ and R₇ are each hydroxy groups; R₂, R₄ and R₅ are hydrogen atoms; and R₃ₐ and R_{3b} are a hydrogen atom, an alkyl group, a cycloalkyl group, an alkaryl group, an aryl group, or an acyl group)).

Referring again to the chemical compound having formula (I), in one further embodiment, three of R₂, R₄, R₅, R₆ and R₇ can be a hydroxy group, wherein the non-hydroxylated R₂, R₅, R₆, or R₇ substituents are a hydrogen atom or an alkyl group or O-alkyl group, and wherein R₄, when it is not a hydroxy group, is a phosphate group, hydrogen atom or an alkyl group or O-alkyl group.

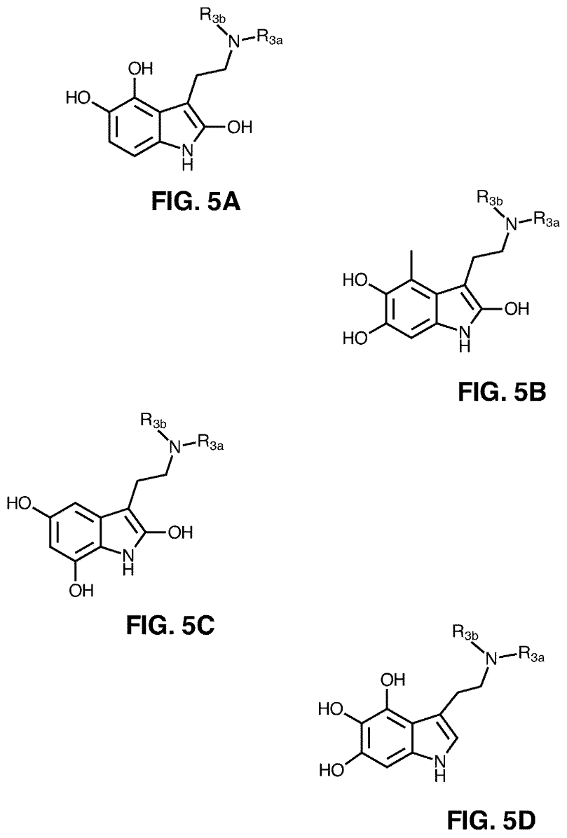

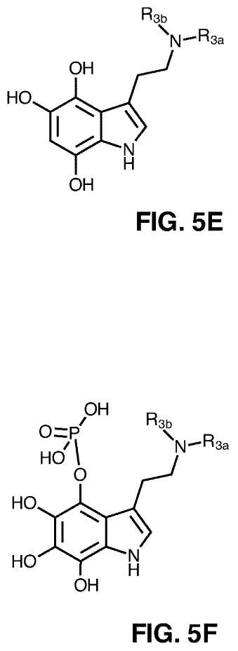

Thus, referring to the chemical compound having formula (I) again, in one embodiment R₂, R₄, and R₅ can be a hydroxy group, and R₆ and R₇ can be a hydrogen atom or an alkyl group or O-alkyl group (see: the example hydroxylated psilocybin derivative shown in FIG. 5A (R₂, R₄ and R₅ are each hydroxy groups; R₆ and R₇ are hydrogen atoms; and R₃ₐ and R_{3b} are a hydrogen atom, an alkyl group, a cycloalkyl group, an alkaryl group, an aryl group, or an acyl group)).

Referring to the chemical compound having formula (I), in one embodiment, R₂, R₅, and R₆ can be a hydroxy groups, and R₇ can be a hydrogen atom or alkyl group, and R₄ can a phosphate group, a hydrogen atom or an alkyl group or O-alkyl group (see: the example hydroxylated psilocybin derivative shown in FIG. 5B (R₂, R₅ and R₆ are each hydroxy groups; R₄ is a methyl group; R₇ is a hydrogen atom; and R₃ₐ and R_{3b} are a hydrogen atom, an alkyl group, a cycloalkyl group, an alkaryl group, an aryl group, or an acyl group)).

Referring to the chemical compound having formula (I), in one embodiment, R₂, R₅, and R₇ can be a hydroxy group and R₆ can be a hydrogen atom or alkyl group, and R₄ can a phosphate group, a hydrogen atom or alkyl group (see: the example hydroxylated psilocybin derivative shown in FIG. 5C (R₂, R₅ and R₇ are each hydroxy groups; R₄ and R₆ are hydrogen atoms; and R₃ₐ and R_{3b} are a hydrogen atom, an alkyl group, a cycloalkyl group, an alkaryl group, an aryl group, or an acyl group)).

Referring to the chemical compound having formula (I), in one embodiment, R₄, R₅, and R₆ can be a hydroxy group, and R₂ and R₇ can be a hydrogen atom or alkyl group (see: the example hydroxylated psilocybin derivative shown in FIG. 5D (R₄, R₅ and R₆ are each a hydroxy group; R₂ and R₇ are hydrogen atoms; and R₃ₐ and R_{3b} are a hydrogen atom, an alkyl group, a cycloalkyl group, an alkaryl group, an aryl group, or an acyl group)).

Referring to the chemical compound having formula (I), in one embodiment, R₄, R₅, and R₇ can be a hydroxy group, and R₂ and R₆ can be a hydrogen atom or alkyl group (see: the example hydroxylated psilocybin derivative shown in FIG. 5E (R₄ R₅ and R₇ are each a hydroxy group; R₂ and R₆ are hydrogen atoms; and R₃ₐ and R_{3b} are a hydrogen atom, an alkyl group, a cycloalkyl group, an alkaryl group, an aryl group, or an acyl group)).

Referring to the chemical compound having formula (I), in one embodiment, R₅, R₆, and R₇ can a hydroxy group, and R₂ can be a hydrogen atom or an alkyl group or O-alkyl group, and R₄ can be a phosphate group, a hydrogen atom or an alkyl group or O-alkyl group (see: the example hydroxylated psilocybin derivative shown in FIG. 5F (R₅, R₆ and R₇ are each a hydroxy group; R₄ is a phosphate group; R₂ is a hydrogen atom; and R₃ₐ and R_{3b} are a hydrogen atom, an alkyl group, a cycloalkyl group, an alkaryl group, an aryl group, or an acyl group)).

Referring again to the chemical compound having formula (I), in one embodiment, four of R₂, R₄, R₅, R₆ and R₇ can be a hydroxy group and wherein the non-hydroxylated R₂, R₅, R₆, or R₇ is a hydrogen atom or an alkyl group or O-alkyl group, and wherein R₄, when it is not a hydroxy group, is a phosphate group, a hydrogen atom or an alkyl group or O-alkyl group.

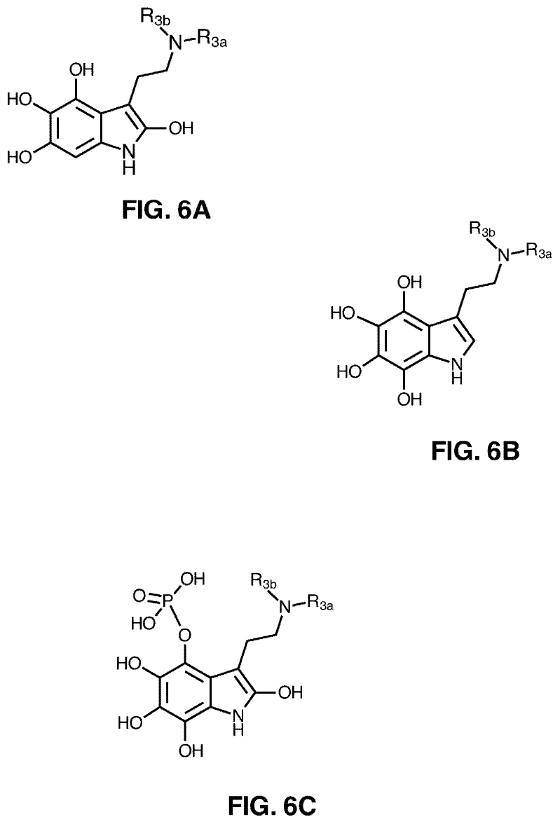

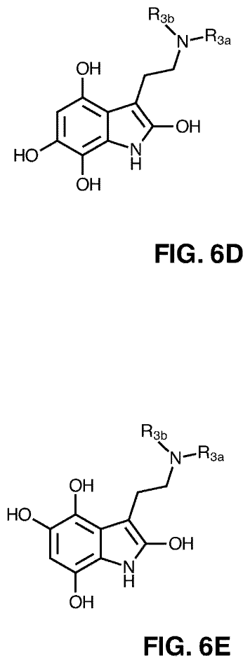

Thus, referring to the chemical compound having formula (I), in one embodiment, R₂, R₄, R₅ and R₆ can be a hydroxy group and R₇ can be a hydrogen atom or an alkyl group or O-alkyl group (see: the example hydroxylated psilocybin derivative shown in FIG. 6A (R₂, R₄, R₅ and R₆ are each hydroxy groups; R₇ is a hydrogen atom; and R₃ₐ and R_{3b} are a hydrogen atom, an alkyl group, a cycloalkyl group, an alkaryl group, an aryl group, or an acyl group)).

Referring to the chemical compound having formula (I), in one embodiment, R₄, R₅, R₆ and R₇ can be a hydroxy group and R₂ can be a hydrogen atom or an alkyl group or O-alkyl group (see: the example hydroxylated psilocybin derivative shown in FIG. 6B (R₄, R₅, R₆ and R₇ are each hydroxy groups; R₂ is a hydrogen atom; and R₃ₐ and R_{3b} are a hydrogen atom, an alkyl group, a cycloalkyl group, an alkaryl group, an aryl group, or an acyl group)).

Referring to the chemical compound having formula (I), in one embodiment, R₂, R₅, R₆ and R₇ can be a hydroxy group, R₄ can be a phosphate group, a hydrogen atom or an alkyl group or O-alkyl group (see: the example hydroxylated psilocybin derivative shown in FIG. 6C (R₂, R₅, R₆ and R₇ are each hydroxy groups; R₄ is a phosphate group; and R₃ₐ and R_{3b} are a hydrogen atom, an alkyl group, a cycloalkyl group, an alkaryl group, an aryl group, or an acyl group)).

Referring to the chemical compound having formula (I), in one embodiment R₂, R₄, R₆ and R₇ can be a hydroxy group and R₅ can be a hydrogen atom or alkyl group (see: the example hydroxylated psilocybin derivative shown in FIG. 6D (R₂, R₄, R₆, and R₇ are hydroxy groups; R₅ is a hydrogen atom; and R₅ a and R_{3b} are a hydrogen atom, an alkyl group, a cycloalkyl group, an alkaryl group, an aryl group, or an acyl group)).

Referring to the chemical compound having formula (I), in one embodiment R₂, R₄, R₅ and R₇ can be hydroxy groups and R₆ can be a hydrogen atom or alkyl group (see: the example hydroxylated psilocybin derivative shown in FIG. 6E (R₂, R₄, R₅ and R₇ are each hydroxy groups; R₆ is a hydrogen atom; and R₃ₐ and R_{3b} are a hydrogen atom, an alkyl group, a cycloalkyl group, an alkaryl group, an aryl group, or an acyl group)).

In one embodiment, all five of R₂, R₄, R₅, R₆ and R₇ can be a hydroxy group.

It is noted that, in a further aspect hereof, R_{3A} and R_{3B} can be a hydrogen atom, an alkyl group, a cycloalkyl group, an alkaryl group, an aryl group or an acyl group. Thus, for example, R_{3A} and R_{3B} can each be a hydrogen atom, or R_{3A} and R_{3B} can each be an alkyl group, such as a methyl group, ethyl group, propyl group, or longer chain alkyl group, or R_{3A} and R_{3B} can each be a cycloalkyl group, such as cyclopentyl group, a cyclohexyl group, or a cycloheptyl group, or R_{3A} and R_{3B} can each be an alkaryl group such as-CH₂CH₂-phenyl, —CH₂CH(CH₃)-phenyl, —CH₂-phenyl, or —CH(CH₃)-phenyl, or R_{3A} and R_{3B} can each be an aryl group, such as a phenyl group or a naphthyl group, or R_{3A} and R_{3B} can each be an acyl group, such as an acetyl group. Furthermore, one of R_{3A} and R_{3B} can be a hydrogen atom, and one of R_{3A} and R_{3B} can be an alkyl group, a cycloalkyl group, an alkaryl group, aryl group, or an acyl group. Furthermore, R_{3A} and R_{3B} can be an aryl group and an alkyl group, an aryl group and an acyl group, or an alkyl group and an acyl group.

In one further embodiment of the disclosure, a chemical compound or salt thereof having formula (I) is included:

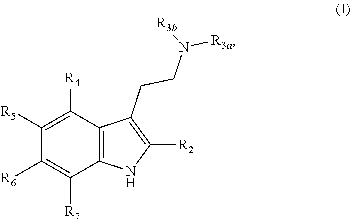

wherein at least one of R₂, R₄, R₅, R₆, or R is a hydroxy group, and wherein each non-hydroxylated R₂, R₅, R₆, or R₇ is a hydrogen atom or an alkyl group or O-alkyl group, wherein R₄ when it is not hydroxylated is a hydrogen atom, alkyl group, or a phosphate group, and wherein R_{3A} and R_{3B} are a hydrogen atom, an alkyl group, a cycloalkyl group, an alkaryl group, an aryl group, or an acyl group.

In one embodiment, at least one of R₂, R₄, R₅, R₆, or R₇ is a hydroxy group, and wherein each non-hydroxylated R₂, R₅, R₆, or R₇ is a hydrogen atom or a (C₁-C₂₀)-alkyl group, wherein R₄ when it is not hydroxylated is a hydrogen atom, a (C₁-C₂₀)-alkyl group, or a phosphate group. In another embodiment, each non-hydroxylated R₂, R₅, R₆, or R₇ is a hydrogen atom or a (C₁-C₁₀)-alkyl group. In another embodiment, each non-hydroxylated R₂, R₅, R₆, or R₇ is a hydrogen atom or a (C₁-C₆)-alkyl group. In another embodiment, each non-hydroxylated R₂, R₅, R₆, or R₇ is a hydrogen atom, a methyl group, an ethyl group or a propyl group.

In another embodiment, when R₄ is not hydroxylated, R₄ is a hydrogen atom, a (C₁-C₂₀)-alkyl group, or a phosphate group. In another embodiment, when R₄ is not hydroxylated, R₄ is a hydrogen atom, a (C₁-C₁₀)-alkyl group, or a phosphate group. In another embodiment, when R₄ is not hydroxylated, R₄ is a hydrogen atom, a (C₁-C₆)-alkyl group, or a phosphate group. In another embodiment, when R₄ is not hydroxylated, R₄ is a hydrogen atom, a methyl group, an ethyl group, a propyl group, or a phosphate group.

In another embodiment, R_{3A} and R_{3B} are a hydrogen atom, a (C₁-C₂₀)-alkyl group, a (C₃-C₂₀)-cycloalkyl group, a (C₁-C₂₀)-alk-(C₆-C₁₄)-aryl group, a (C₆-C₁₄)-aryl group, or a —C(═O)(C₁-C₂₀)-alkyl group. In another embodiment, R_{3A} and R_{3B} are a hydrogen atom, a (C₁-C₁₀)-alkyl group, a (C₃-C₁₀)-cycloalkyl group, a (C₁-C₁₀)-alk-(C₆-C₁₀)-aryl group a (C₆-C₁₀)-aryl group, or a —C(═O)(C₁-C₁₀)-alkyl group. In another embodiment, R_{3A} and R_{3B} are a hydrogen atom, a (C₁-C₆)-alkyl group, a (C₃-C₇)-cycloalkyl group, a (C₁-C₆)-alk-phenyl group, a phenyl group, or a —C(═O)(C₁-C₆)-alkyl group. In another embodiment, R_{3A} and R_{3B} are a hydrogen atom, a methyl group, an ethyl group, a propyl group, a cyclopentyl group, a cyclohexyl group, a cycloheptyl group, —CH₂CH₂-phenyl, —CH₂CH(CH₃)-phenyl, —CH₂-phenyl, —CH(CH₃)-phenyl, a phenyl group, —C(═O)—CH₃, —C(═O)—CH₂CH₃, or —C(═O)—CH₂CH₂CH₃.

In one embodiment of the disclosure, a chemical compound or salt thereof having formula (I) is included:

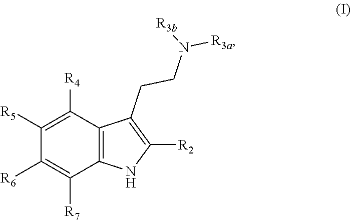

wherein
R₂, R₅, R₆, and R₇ are independently or simultaneously H, an alkyl group or a hydroxy group, R_{3A} and R_{3B} are a hydrogen atom, an alkyl group, a cycloalkyl group, an alkaryl group, an aryl group, or an acyl group; and R₄ is hydrogen atom, alkyl group, a hydroxy group or a phosphate group;
wherein at least one of R₂, R₄ R₅, R₆, and R₇ is a hydroxy group.

In one embodiment, R₂, R₅, R₆, and R₇ are independently or simultaneously H, (C₁-C₂₀)-alkyl group or a hydroxy group. In one embodiment, R₂, R₅, R₆, and R₇ are independently or simultaneously H, (C₁-C₁₀)-alkyl group or a hydroxy group. In one embodiment, R₂, R₅, R₆, and R₇ are independently or simultaneously H, (C₁-C₆)-alkyl group or a hydroxy group. In one embodiment, R₂, R₅, R₆, and R₇ are independently or simultaneously H, methyl, ethyl, propyl or a hydroxy group.

In one embodiment, R₄ is H, (C₁-C₂₀)-alkyl group, a hydroxy group or a phosphate group. In one embodiment, R₄ is H, (C₁-C₁₀)-alkyl group, a hydroxy group or a phosphate group. In one embodiment, R₄ is H, (C₁-C₆)-alkyl group, a hydroxy group or a phosphate group. In one embodiment, R₄ is H, methyl, ethyl, propyl, a hydroxy group or a phosphate group.

In another embodiment, R_{3A} and R_{3B} are a hydrogen atom, a (C₁-C₂₀)-alkyl group, a (C₃-C₂₀)-cycloalkyl group, a (C₁-C₂₀)-alk-(C₆-C₁₄)-aryl group, a (C₆-C₁₄)-aryl group, or a —C(═O)(C₁-C₂₀)-alkyl group. In another embodiment, R_{3A} and R_{3B} are a hydrogen atom, a (C₁-C₁₀)-alkyl group, a (C₃-C₁₀)-cycloalkyl group, a (C₁-C₁₀)-alk-(C₆-C₁₀)-aryl group a (C₆-C₁₀)-aryl group, or a —C(═O)(C₁-C₁₀)-alkyl group. In another embodiment, R_{3A} and R_{3B} are a hydrogen atom, a (C₁-C₆)-alkyl group, a (C₃-C₇)-cycloalkyl group, a (C₁-C₆)-alk-phenyl group, a phenyl group, or a —C(═O)(C₁-C₆)-alkyl group. In another embodiment, R_{3A} and R_{3B} are a hydrogen atom, a methyl group, an ethyl group, a propyl group, a cyclopentyl group, a cyclohexyl group, a cycloheptyl group, —CH₂CH₂-phenyl, —CH₂CH(CH₃)-phenyl, —CH₂-phenyl, —CH(CH₃)-phenyl, a phenyl group, —C(═O)—CH₃, —C(═O)—CH₂CH₃, or —C(═O)—CH₂CH₂CH₃.

Furthermore, in one embodiment, a hydroxylated psilocybin derivative according to the present disclosure can be a chemical compound having the formula (III):

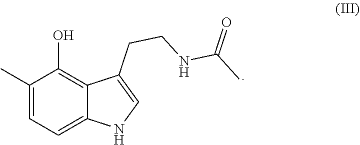 [SMILES: CC(=O)NCCc1c[nH]c2ccc(C)c(O)c12]

Furthermore, in one embodiment, a hydroxylated psilocybin derivative according to the present disclosure can be a chemical compound having the formula (IV):

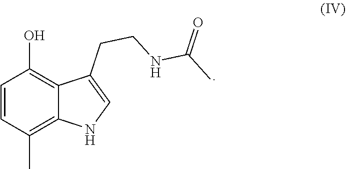 [SMILES: CC(=O)NCCc1c[nH]c2c(C)ccc(O)c12]

Furthermore, in one embodiment, a hydroxylated psilocybin derivative according to the present disclosure can be a chemical compound having the formula (V):

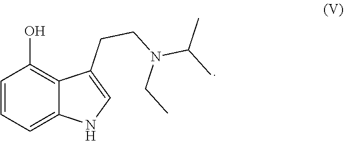 [SMILES: CCN(CCc1c[nH]c2cccc(O)c12)C(C)C]

Furthermore, in one embodiment, a hydroxylated psilocybin derivative according to the present disclosure can be a chemical compound having the formula (VI):

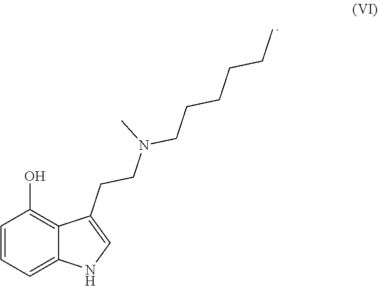 [SMILES: CCCCCCN(C)CCc1c[nH]c2cccc(O)c12]

Furthermore, in one embodiment, a hydroxylated psilocybin derivative according to the present disclosure can be a chemical compound having the formula (VII):

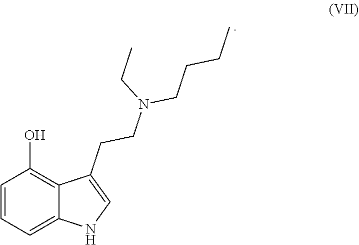 [SMILES: CCCCN(CC)CCc1c[nH]c2cccc(O)c12]

Furthermore, in one embodiment, a hydroxylated psilocybin derivative according to the present disclosure can be a chemical compound having the formula (VIII):

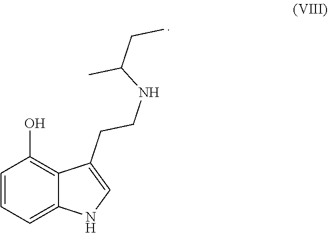 [SMILES: CCC(C)NCCc1c[nH]c2cccc(O)c12]

Furthermore, in one embodiment, a hydroxylated psilocybin derivative according to the present disclosure can be a chemical compound having the formula (IX):

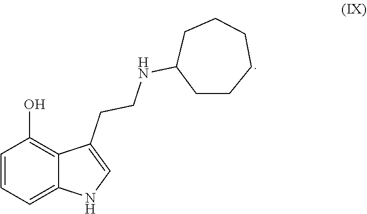 [SMILES: Oc1cccc2[nH]cc(CCNC3CCCCCC3)c12]

Furthermore, in one embodiment, a hydroxylated psilocybin derivative according to the present disclosure can be a chemical compound having the formula (X):

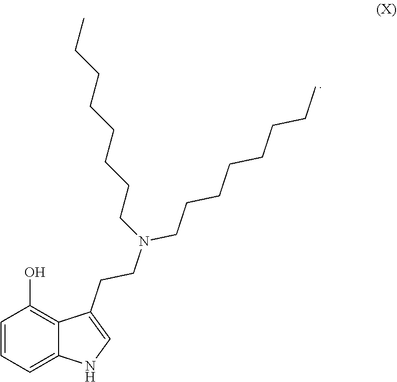 [SMILES: CCCCCCCCN(CCCCCCCC)CCc1c[nH]c2cccc(O)c12]

Furthermore, in one embodiment, a hydroxylated psilocybin derivative according to the present disclosure can be a chemical compound having the formula (XI):

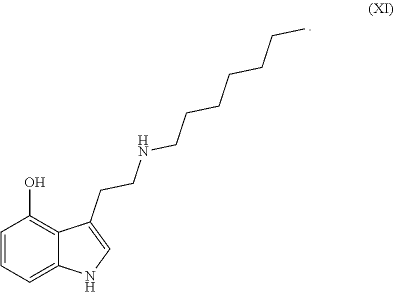 [SMILES: CCCCCCCNCCc1c[nH]c2cccc(O)c12]

Furthermore, in one embodiment, a hydroxylated psilocybin derivative according to the present disclosure can be a chemical compound having the formula (XII):

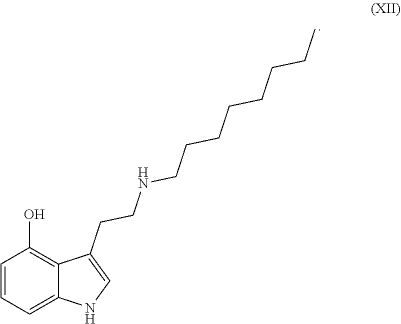 [SMILES: CCCCCCCCNCCc1c[nH]c2cccc(O)c12]

Furthermore, in one embodiment, a hydroxylated psilocybin derivative according to the present disclosure can be a chemical compound having the formula (XIII):

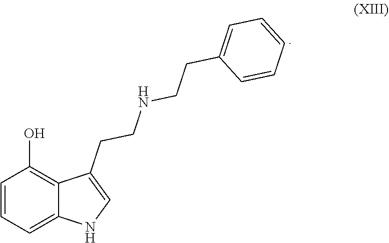 [SMILES: Oc1cccc2[nH]cc(CCNCCc3ccccc3)c12]

Furthermore, in one embodiment, a hydroxylated psilocybin derivative according to the present disclosure can be a chemical compound having the formula (XIV):

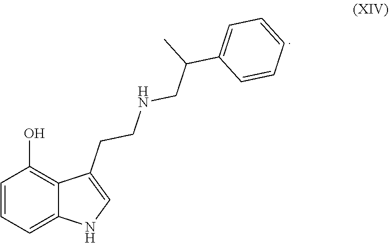 [SMILES: CC(CNCCc1c[nH]c2cccc(O)c12)c1ccccc1]

Furthermore, in one embodiment, a hydroxylated psilocybin derivative according to the present disclosure can be a chemical compound having the formula (XV):

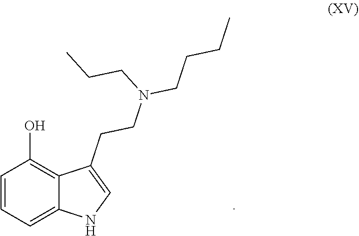 [SMILES: CCCCN(CCC)CCc1c[nH]c2cccc(O)c12]

Furthermore, in one embodiment, a hydroxylated psilocybin derivative according to the present disclosure can be a chemical compound having the formula (XVI):

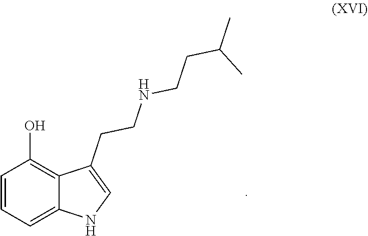 [SMILES: CC(C)CCNCCc1c[nH]c2cccc(O)c12]

Furthermore, in one embodiment, a hydroxylated psilocybin derivative according to the present disclosure can be a chemical compound having the formula (XVII):

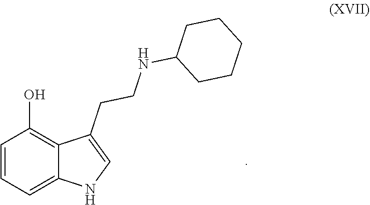 [SMILES: Oc1cccc2[nH]cc(CCNC3CCCCC3)c12]

Furthermore, in one embodiment, a hydroxylated psilocybin derivative according to the present disclosure can be a chemical compound having the formula (XVIII):

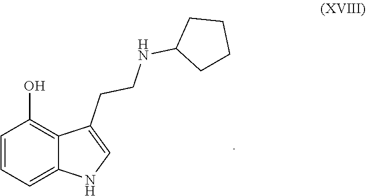 [SMILES: Oc1cccc2[nH]cc(CCNC3CCCC3)c12]

Furthermore, in one embodiment, a hydroxylated psilocybin derivative according to the present disclosure can be a chemical compound having the formula (XIX):

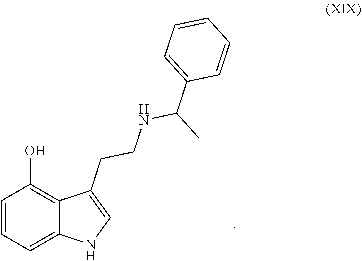 [SMILES: CC(NCCc1c[nH]c2cccc(O)c12)c1ccccc1]

Furthermore, in one embodiment, a hydroxylated psilocybin derivative according to the present disclosure can be a chemical compound having the formula (XX):

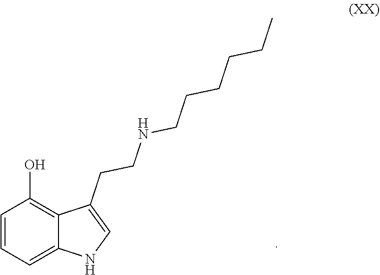 [SMILES: CCCCCCNCCc1c[nH]c2cccc(O)c12]

Furthermore, in one embodiment, a hydroxylated psilocybin derivative according to the present disclosure can be a chemical compound having the formula (XXI):

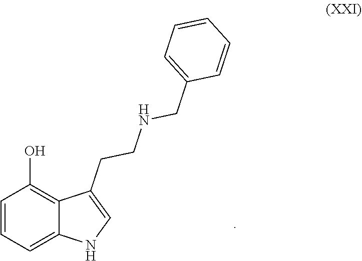 [SMILES: Oc1cccc2[nH]cc(CCNCc3ccccc3)c12]

Furthermore, in one embodiment, a hydroxylated psilocybin derivative according to the present disclosure can be a chemical compound having the formula (XXII):

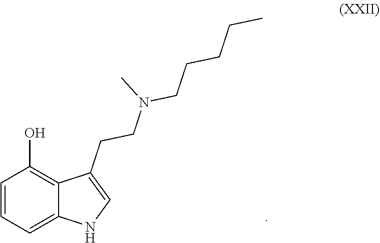 [SMILES: CCCCCN(C)CCc1c[nH]c2cccc(O)c12]

Furthermore, in one embodiment, a hydroxylated psilocybin derivative according to the present disclosure can be a chemical compound having the formula (XXIII):

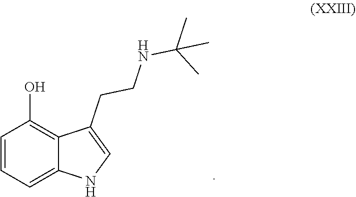 [SMILES: CC(C)(C)NCCc1c[nH]c2cccc(O)c12]

Furthermore, in one embodiment, a hydroxylated psilocybin derivative according to the present disclosure can be a chemical compound having the formula (XXIV):

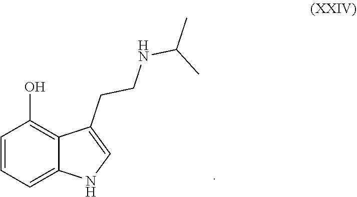 [SMILES: CC(C)NCCc1c[nH]c2cccc(O)c12]

Furthermore, in one embodiment, a hydroxylated psilocybin derivative according to the present disclosure can be a chemical compound having the formula (XXV):

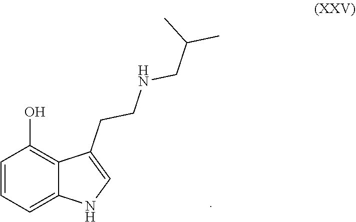 [SMILES: CC(C)CNCCc1c[nH]c2cccc(O)c12]

Furthermore, in one embodiment, a hydroxylated psilocybin derivative according to the present disclosure can be a chemical compound having the formula (XXVI):

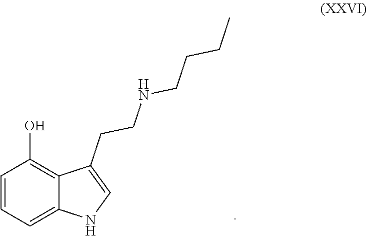 [SMILES: CCCCNCCc1c[nH]c2cccc(O)c12]

Furthermore, in one embodiment, a hydroxylated psilocybin derivative according to the present disclosure can be a chemical compound having the formula (XXVII):

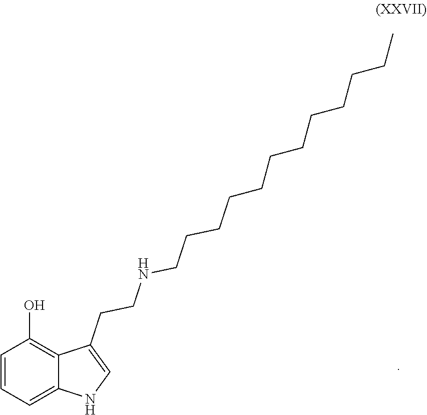 [SMILES: CCCCCCCCCCCCNCCc1c[nH]c2cccc(O)c12]

Furthermore, in one embodiment, a hydroxylated psilocybin derivative according to the present disclosure can be a chemical compound having the formula (XXVIII):

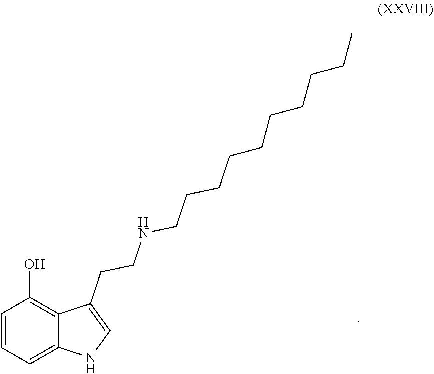 [SMILES: CCCCCCCCCCNCCc1c[nH]c2cccc(O)c12]

Furthermore, in one embodiment, a hydroxylated psilocybin derivative according to the present disclosure can be a chemical compound having the formula (XXIX):

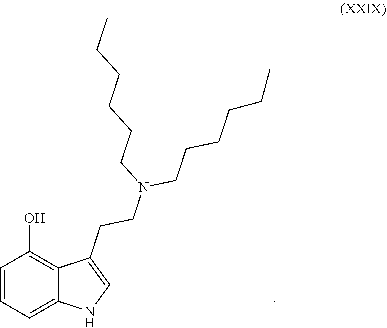 [SMILES: CCCCCCN(CCCCCC)CCc1c[nH]c2cccc(O)c12]

Furthermore, in one embodiment, a hydroxylated psilocybin derivative according to the present disclosure can be a chemical compound having the formula (XXX):

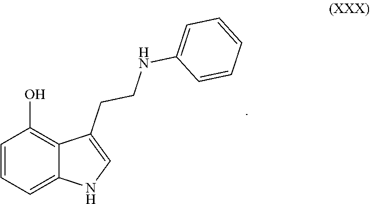 [SMILES: Oc1cccc2[nH]cc(CCNc3ccccc3)c12]

Furthermore, in one embodiment, a hydroxylated psilocybin derivative according to the present disclosure can be a chemical compound having the formula (XXXI):

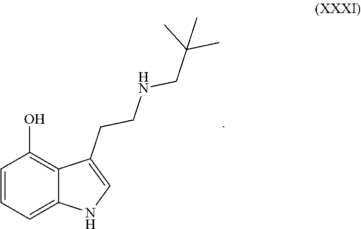 [SMILES: CC(C)(C)CNCCc1c[nH]c2cccc(O)c12]

Furthermore, in one embodiment, a hydroxylated psilocybin derivative according to the present disclosure can be a chemical compound having the formula (XXXII):

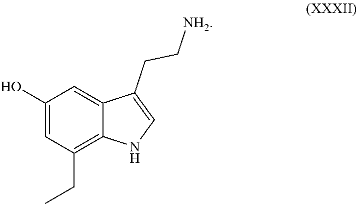

Furthermore, it is noted that the hydroxylated psilocybin derivatives of the present disclosure include salts thereof, including pharmaceutically acceptable salts. Thus, the nitrogen atom of the 2-aminoethyl group extending in turn from the C₃ atom may be protonated, and the positive charge may be balanced by, for example, chloride or sulfate ions, to thereby form a chloride salt or a sulfate salt. Furthermore, in compounds wherein R₄ is a phosphate group, the phosphate group may be de-protonated, and the negative charge may be balanced by, for example, sodium ions or potassium ions, to thereby form a sodium salt or a potassium salt.

Furthermore, it is noted that when R₄ is a phosphate group, the term hydroxylated psilocybin derivative also includes compounds having the formula (XXXIII):

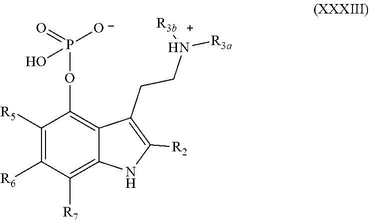

wherein at least one of R₂, R₅, R₆, or R₇ is a hydroxy group, and wherein any R₂, R₅, R₆, or R₇ which are not a hydroxy group are a hydrogen atom or an alkyl group or O-alkyl group, and wherein R_{3A} and R_{3B} are a hydrogen atom, an alkyl group, a cycloalkyl group, an alkaryl group, and aryl group or an acyl group. Further included are salts of hydroxylated psilocybins having the formula (VII), such as a sodium salt, a potassium salt etc.

Thus, to briefly recap, the present disclosure provides hydroxylated psilocybin derivatives. The disclosure provides, in particular, a chemical compound or salt thereof having formula (I):

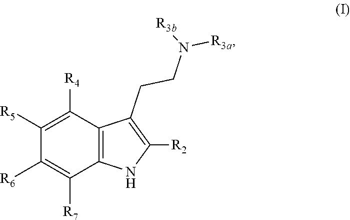

wherein in an aspect, at least one of R₂, R₄, R₅, R₆, or R₇ is a hydroxy group. In an aspect, in formula (I), each non-hydroxylated R₂, R₅, R₆, or R₇ is a hydrogen atom or an alkyl group or O-alkyl group. In a further aspect, in formula (I), R₄ when it is not hydroxylated is a hydrogen atom, an alkyl group or a phosphate group. Yet in a further aspect, R_{3A} and R_{3B} are a hydrogen atom, an alkyl group, a cycloalkyl group, an alkaryl group, an aryl group, or an acyl group.

In one embodiment of the disclosure, a chemical compound or salt thereof having formula (I) is included:

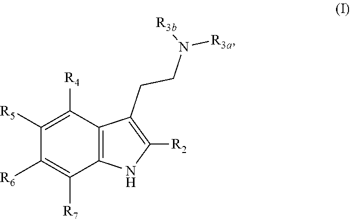

wherein at least one of R₂, R₄, R₅, R₆, or R₇ is a hydroxy group, and wherein each non-hydroxylated R₂, R₅, R₆, or R₇ is a hydrogen atom or an alkyl group or O-alkyl group, wherein R₄ when it is not hydroxylated is a hydrogen atom, an alkyl group or a phosphate group, and wherein R₅ A and R_{3B} are a hydrogen atom an alkyl group, a cycloalkyl group, an alkaryl group, an aryl group, or an acyl group.

In one embodiment, at least one of R₂, R₄, R₅, R₆, or R₇ is a hydroxy group, and wherein each non-hydroxylated R₂, R₅, R₆, or R₇ is a hydrogen atom or a (C₁-C₂₀)-alkyl group or (C₁-C₂₀)—O-alkyl group, wherein R₄ when it is not hydroxylated is a hydrogen atom, a (C₁-C₂₀)-alkyl group or (C₁-C₂₀)—O-alkyl group, or a phosphate group.

In another embodiment, each non-hydroxylated R₂, R₅, R₆, or R₇ is a hydrogen atom or a (C₁-C₁₀)-alkyl group or (C₁-C₁₀)—O-alkyl group. In another embodiment, each non-hydroxylated R₂, R₅, R₆, or R₇ is a hydrogen atom or a (C₁-C₆)-alkyl group or (C₁-C₆)—O-alkyl group. In another embodiment, each non-hydroxylated R₂, R₅, R₆, or R₇ is a hydrogen atom, a methyl group, ethyl group, a propyl group, an O-methyl group, an O-ethyl group, or an O-propyl group.

In another embodiment, when R₄ is not hydroxylated, R₄ is a hydrogen atom, a (C₁-C₂₀)-alkyl group or (C₁-C₂₀)—O-alkyl group, or a phosphate group. In another embodiment, when R₄ is not hydroxylated, R₄ is a hydrogen atom, a (C₁-C₁₀)-alkyl group or (C₁-C₁₀)—O-alkyl group, or a phosphate group. In another embodiment, when R₄ is not hydroxylated, R₄ is a hydrogen atom, a (C₁-C₆)-alkyl group or (C₁-C₆)—O-alkyl group, or a phosphate group. In another embodiment, when R₄ is not hydroxylated, R₄ is a hydrogen atom, a methyl group, an ethyl group, a propyl group, a phosphate group, an O-methyl group, an O-ethyl group, or an O-propyl group.

In another embodiment, R_{3A} and R_{3B} are a hydrogen atom, a (C₁-C₂₀)-alkyl group, a (C₃-C₂₀)-cycloalkyl group, a (C₁-C₂₀)-alk-(C₆-C₁₄)-aryl group, a (C₆-C₁₄)-aryl group, or a —C(═O)(C₁-C₂₀)-alkyl group. In another embodiment, R_{3A} and R_{3B} are a hydrogen atom, a (C₁-C₁₀)-alkyl group, a (C₃-C₁₀)-cycloalkyl group, a (C₁-C₁₀)-alk-(C₆-C₁₀)-aryl group a (C₆-C₁₀)-aryl group, or a —C(═O)(C₁-C₁₀)-alkyl group. In another embodiment, R_{3A} and R_{3B} are a hydrogen atom, a (C₁-C₆)-alkyl group, a (C₃-C₇)-cycloalkyl group, a (C₁-C₆)-alk-phenyl group, a phenyl group, or a —C(═O)(C₁-C₆)-alkyl group. In another embodiment, R₃ and R_{3B} are a hydrogen atom, a methyl group, an ethyl group, a propyl group, a cyclopentyl group, a cyclohexyl group, a cycloheptyl group, —CH₂CH₂-phenyl, —CH₂CH(CH₃)-phenyl, —CH₂-phenyl, —CH(CH₃)-phenyl, a phenyl group, —C(═O)—CH₃, —C(═O)—CH₂CH₃, or —C(═O)—CH₂CH₂CH₃.

In one embodiment of the disclosure, a chemical compound or salt thereof having formula (I) is included:

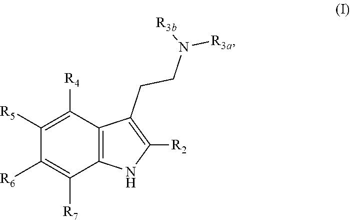

wherein
R₂, R₅, R₆, and Rare independently or simultaneously H, an alkyl group or O-alkyl group or a hydroxy group, R_{3A} and R_{3B} are a hydrogen atom, an alkyl group, a cycloalkyl group, an alkaryl group, an aryl group, or an acyl group; and R₄ is hydrogen atom, alkyl group or O-alkyl group, a hydroxy group or a phosphate group; wherein at least one of R₂, R₄ R₅, R₆, and R₇ is a hydroxy group.

In one embodiment, R₂, R₅, R₆, and R₇ are independently or simultaneously H, (C₁-C₂₀)-alkyl group or (C₁-C₂₀)—O-alkyl group or a hydroxy group. In one embodiment, R₂, R₅, R₆, and Rare independently or simultaneously H, (C₁-C₁₀)-alkyl group or (C₁-C₁₀)—O-alkyl group or a hydroxy group. In one embodiment, R₂, R₅, R₆, and R₇ are independently or simultaneously H, (C₁-C₆)-alkyl group or (C₁-C₆)—O-alkyl group or a hydroxy group. In one embodiment, R₂, R₅, R₆, and R₇ are independently or simultaneously H, methyl, ethyl, propyl, O-methyl, O-ethyl, O-propyl, or a hydroxy group.

In one embodiment, R₄ is H, (C₁-C₂₀)-alkyl group or (C₁-C₂₀)—O-alkyl group, a hydroxy group or a phosphate group. In one embodiment, R₄ is H, (C₁-C₁₀)-alkyl group or (C₁-C₁₀)—O-alkyl group, a hydroxy group or a phosphate group. In one embodiment, R₄ is H, (C₁-C₆)-alkyl group or (C₁-C₆)—O-alkyl group, a hydroxy group or a phosphate group. In one embodiment, R₄ is H, methyl, ethyl, propyl, O-methyl, O-ethyl, O-propyl, a hydroxy group, or a phosphate group.

In another embodiment, R_{3A} and R_{3B} are a hydrogen atom, a (C₁-C₂₀)-alkyl group, a (C₃-C₂₀)-cycloalkyl group, a (C₁-C₂₀)-alk-(C₆-C₁₄)-aryl group, a (C₆-C₁₄)-aryl group, or a —C(═O)(C₁-C₂₀)-alkyl group. In another embodiment, R_{3A} and R_{3B} are a hydrogen atom, a (C₁-C₁₀)-alkyl group, a (C₃-C₁₀)-cycloalkyl group, a (C₁-C₁₀)-alk-(C₆-C₁₀)-aryl group a (C₆-C₁₀)-aryl group, or a —C(═O)(C₁-C₁₀)-alkyl group. In another embodiment, R_{3A} and R_{3B} are a hydrogen atom, a (C₁-C₆)-alkyl group, a (C₃-C₇)-cycloalkyl group, a (C₁-C₆)-alk-phenyl group, a phenyl group, or a —C(═O)(C₁-C₆)-alkyl group. In another embodiment, R_{3A} and R_{3B} are a hydrogen atom, a methyl group, an ethyl group, a propyl group, a cyclopentyl group, a cyclohexyl group, a cycloheptyl group, —CH₂CH₂-phenyl, —CH₂CH(CH₃)-phenyl, —CH₂-phenyl, —CH(CH₃)-phenyl, a phenyl group, —C(═O)—CH₃, —C(═O)—CH₂CH₃, or —C(═O)—CH₂CH₂CH₃.

The hydroxylated psilocybin derivatives of the present disclosure may be used to prepare a pharmaceutical or recreational drug formulation. Thus in one embodiment, the present disclosure further provides in another aspect, pharmaceutical and recreational drug formulations comprising hydroxylated psilocybin derivatives. Accordingly, in one aspect, the present disclosure provides in a further embodiment a pharmaceutical or recreational drug formulation comprising a chemical compound having formula (I):

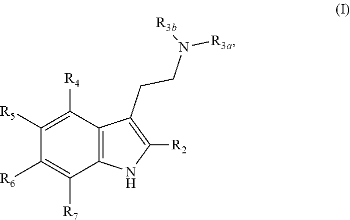

wherein at least one of R₂, R₄, R₅, R₆, or R₇ is a hydroxy group, and wherein each non-hydroxylated R₂, R₅, R₆, or R₇ is a hydrogen atom or an alkyl group or O-alkyl group, wherein R₄ when it is not hydroxylated is a hydrogen atom, an alkyl group or a phosphate group, and wherein R_{3A} and R_{3B} are a hydrogen atom an alkyl group, a cycloalkyl group, an alkaryl group, an aryl group, or an acyl group, or a salt of the chemical compound, together with a diluent, carrier or excipient.

The dose when using the compounds of the present disclosure can vary within wide limits, and as is customary and is known to those of skill in the art, the dose can be tailored to the individual conditions in each individual case. The dose depends, for example, on the nature and severity of the illness to be treated, on the condition of the patient, on the compound employed or on whether an acute or chronic disease state is treated or prophylaxis is conducted, on the mode of delivery of the compound, or on whether further active compounds are administered in addition to the compounds of the present disclosure. Representative doses of the present invention include, but are not limited to, about 0.001 mg to about 5000 mg, about 0.001 mg to about 2500 mg, about 0.001 mg to about 1000 mg, about 0.001 mg to about 500 mg, about 0.001 mg to about 250 mg, about 0.001 mg to about 100 mg, about 0.001 mg to about 50 mg, and about 0.001 mg to about 25 mg. Representative doses of the present disclosure include, but are not limited to, about 0.0001 to about 1,000 mg, about 10 to about 160 mg, about 10 mg, about 20 mg, about 40 mg, about 80 mg or about 160 mg. Multiple doses may be administered during the day, especially when relatively large amounts are deemed to be needed, for example 2, 3 or 4, doses. Depending on the subject and as deemed appropriate from the patient's physician or care giver it may be necessary to deviate upward or downward from the doses described herein.

The pharmaceutical or recreational drug formulations may be prepared as liquids, tablets, capsules, microcapsules, nanocapsules, trans-dermal patches, gels, foams, oils, aerosols, nanoparticulates, powders, creams, emulsions, micellar systems, films, sprays, ovules, infusions, teas, decoctions, suppositories, etc. and include a pharmaceutically acceptable salt or solvate of the hydroxylated psilocybin compound together with an excipient. The term “excipient” as used herein means any ingredient other than the chemical compound of the disclosure. As will readily be appreciated by those of skill in art, the selection of excipient may depend on factors such as the particular mode of administration, the effect of the excipient on solubility of the chemical compounds of the present disclosure and methods for their preparation will be readily apparent to those skilled in the art. Such compositions and methods for their preparation may be found, for example, in “Remington's Pharmaceutical Sciences”, 22nd Edition (Pharmaceutical Press and Philadelphia College of Pharmacy at the University of the Sciences, 2012).

The pharmaceutical and drug formulations comprising the hydroxylated psilocybin derivatives of the present disclosure may be administered orally. Oral administration may involve swallowing, so that the compound enters the gastrointestinal tract, or buccal or sublingual administration may be employed by which the compound enters the blood stream directly from the mouth. Formulations suitable for oral administration include both solid and liquid formulations.

Solid formulations include tablets, capsules (containing particulates, liquids, microcapsules, or powders), lozenges (including liquid-filled lozenges), chews, multi- and nano-particulates, gels, solid solutions, liposomal preparations, microencapsulated preparations, creams, films, ovules, suppositories and sprays.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinized starch, hydroxypropyl cellulose and hydroxypropyl methylcellulose.

Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80. When present, surface active agents may comprise from 0.2% (w/w) to 5% (w/w) of the tablet.

Tablets may further contain lubricants such as magnesium stearate, calcium stearate, zinc stearate, sodium stearyl fumarate, and mixtures of magnesium stearate with sodium lauryl sulphate. Lubricants generally comprise from 0.25% (w/w) to 10% (w/w), from 0.5% (w/w) to 3% (w/w) of the tablet.

In addition to the hydroxylated psilocybin derivative, tablets may contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinized starch and sodium alginate. Generally, the disintegrant will comprise from 1% (w/w) to 25% (w/w) or from 5% (w/w) to 20% (w/w) of the dosage form.

Other possible auxiliary ingredients i include anti-oxidants, colourants, flavouring agents, preservatives and taste-masking agents.

For tablet dosage forms, depending on the desired effective amount of the chemical compound, the chemical compound of the present disclosure may make up from 1% (w/w) to 80% (w/w) of the dosage form, more typically from 5% (w/w) to 60% (w/w) of the dosage form.

Exemplary tablets contain up to about 80% (w/w) of the chemical compound, from about 10% (w/w) to about 90% (w/w) binder, from about 0% (w/w) to about 85% (w/w) diluent, from about 2% (w/w) to about 10% (w/w) disintegrant, and from about 0.25% (w/w) to about 10% (w/w) lubricant.

The formulation of tablets is discussed in “Pharmaceutical Dosage Forms: Tablets”, Vol. 1-Vol. 3, by CRC Press (2008).

The pharmaceutical and recreational drug formulations comprising the hydroxylated psilocybin derivatives of the present disclosure may also be administered directly into the blood stream, into muscle, or into an internal organ. Thus, the pharmaceutical and recreational drug formulations can be administered parenterally (for example, by subcutaneous, intravenous, intraarterial, intrathecal, intraventricular, intracranial, intramuscular, or intraperitoneal injection). Parenteral formulations are typically aqueous solutions which may contain excipients such as salts, carbohydrates and buffering agents (in one embodiment, to a pH of from 3 to 9), but, for some applications, they may be more suitably formulated as a sterile non-aqueous solution or as a dried form to be used in conjunction with a suitable vehicle such as sterile water.

Formulations comprising the hydroxylated psilocybin derivatives of the present disclosure for parenteral administration may be formulated to be immediate and/or modified release. Modified release formulations include delayed-, sustained-, pulsed-, controlled-, targeted and programmed release. Thus the chemical compounds of the disclosure may be formulated as a solid, semi-solid, or thixotropic liquid for administration as an implanted depot providing modified release of the active compound. Examples of such formulations include drug-coated stents and poly(dl-lactic-coglycolic)acid (PGLA) microspheres.

The pharmaceutical or recreational drug formulations of the present disclosure also may be administered topically to the skin or mucosa, i.e. dermally or transdermally. Example pharmaceutical and recreational drug formulations for this purpose include gels, hydrogels, lotions, solutions, creams, ointments, dusting powders, cosmetics, oils, eye drops, dressings, foams, films, skin patches, wafers, implants, sponges, fibres, bandages and microemulsions. Liposomes may also be used. Example carriers include alcohol, water, mineral oil, liquid petrolatum, white petrolatum, glycerin, polyethylene glycol and propylene glycol. Penetration enhancers may be incorporate (see: for example, Finnin, B. and Morgan, T. M., 1999 J. Pharm. Sci, 88 (10), 955-958).

Other means of topical administration include delivery by electroporation, iontophoresis, phonophoresis, sonophoresis and microneedle or needle-free (e.g., Powderject™, Bioject™, etc.) injection.

Pharmaceutical and recreational drug formulations for inhalation or insufflation include solutions and suspensions in pharmaceutically acceptable, aqueous or organic solvents, or mixtures thereof, and powders. The liquid or solid pharmaceutical compositions can contain suitable pharmaceutically acceptable excipients. In some embodiments, the pharmaceutical compositions are administered by the oral or nasal respiratory route for local or systemic effect. Pharmaceutical compositions in pharmaceutically acceptable solvents can be nebulized by use of inert gases. Nebulized solutions can be inhaled directly from the nebulizing device or the nebulizing device can be attached to a face mask tent, or intermittent positive pressure breathing machine. Solution, suspension, or powder pharmaceutical compositions can be administered, e.g., orally or nasally, from devices that deliver the formulation in an appropriate manner.

In further embodiments, in which the hydroxylated psilocybin compounds of present disclosure are used as a recreational drug, the compounds may be included in compositions such as a food or food product, a beverage, a food seasoning, a personal care product, such as a cosmetic, perfume or bath oil, or oils (both for topical administration as massage oil, or to be burned or aerosolized). The chemical compounds of the present disclosure may also be included in a “vape” product, which may also include other drugs, such as nicotine, and flavorings.

The pharmaceutical formulations comprising the chemical compounds of the present disclosure may be used to treat a subject, and in particular to treat a psychiatric disorder in a subject. Accordingly, the present disclosure includes in a further embodiment, a method for treating a psychiatric disorder, the method comprising administering to a subject in need thereof a pharmaceutical formulation comprising a chemical compound or salt thereof having formula (I):

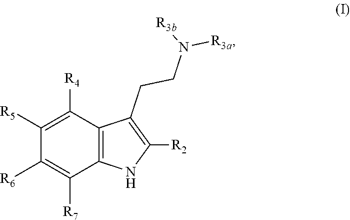

wherein at least one of R₂, R₄, R₅, R₆, or R₇ is a hydroxy group, and wherein each non-hydroxylated R₂, R₅, R₆, or R₇ is a hydrogen atom or an alkyl group or O-alkyl group, wherein R₄ when it is not hydroxylated is a hydrogen atom, an alkyl group or a phosphate group, and wherein R_{3A} and R_{3B} are a hydrogen atom, an alkyl group, a cycloalkyl group, an alkaryl group, an aryl group, or an acyl group, together with a diluent, carrier or excipient.

Psychiatric disorders that may be treated include, for example, neurodevelopmental disorders such as intellectual disability, global development delay, communication disorders, autism spectrum disorder, and attention-deficit hyperactivity disorder (ADHD); bipolar and related disorders, such as mania, and depressive episodes; anxiety disorder, such as generalized anxiety disorder (GAD), agoraphobia, social anxiety disorder, specific phobias (natural events, medical, animal, situational, for example), panic disorder, and separation anxiety disorder; stress disorders, such as acute stress disorder, adjustment disorders, post-traumatic stress disorder (PTSD), and reactive attachment disorder; dissociative disorders, such as dissociative amnesia, dissociative identity disorder, and depersonalization/derealization disorder; somatoform disorders, such as somatic symptom disorders, illness anxiety disorder, conversion disorder, and factitious disorder; eating disorders, such as anorexia nervosa, bulimia nervosa, rumination disorder, pica, and binge-eating disorder; sleep disorders, such as narcolepsy, insomnia disorder, hypersomnolence, breathing-related sleep disorders, parasomnias, and restless legs syndrome; disruptive disorders, such as kleptomania, pyromania, intermittent explosive disorder, conduct disorder, and oppositional defiant disorder; depressive disorders, such as disruptive mood dysregulation disorder, major depressive disorder, persistent depressive disorder (dysthymia), premenstrual dysphoric disorder, substance/medication-induced depressive disorder, postpartum depression, and depressive disorder caused by another medical condition, for example, psychiatric and existential distress within life-threatening cancer situations (ACS Pharmacol. Transl. Sci. 4:553-562; J Psychiatr Res 137:273-282); substance-related disorders, such as alcohol-related disorders, Cannabis related disorders, inhalant-use related disorders, stimulant use disorders, and tobacco use disorders; neurocognitive disorders, such as delirium; schizophrenia; compulsive disorders, such as obsessive compulsive disorders (OCD), body dysmorphic disorder, hoarding disorder, trichotillomania disorder, excoriation disorder, substance/medication induced obsessive-compulsive disorder, and obsessive-compulsive disorder related to another medical condition; and personality disorders, such as antisocial personality disorder, avoidant personality disorder, borderline personality disorder, dependent personality disorder, histrionic personality disorder, narcissistic personality disorder, obsessive-compulsive personality disorder, paranoid personality disorder, schizoid personality disorder, and schizotypal personality disorder.

In an aspect, the compounds of the present disclosure may be used to be contacted with a 5-HT_{2A} receptor to thereby modulate the 5-HT_{2A} receptor. Such contacting includes bringing a compound of the present disclosure and 5-HT_{2A} receptor together under in vitro conditions, for example, by introducing the compounds in a sample containing a 5-HT_{2A} receptor, for example, a sample containing purified 5-HT_{2A} receptors, or a sample containing cells comprising 5-HT_{2A} receptors. In vitro conditions further include the conditions described in Example 3 hereof. Contacting further includes bringing a compound of the present disclosure and 5-HT_{2A} receptor together under in vivo conditions. Such in vivo conditions include the administration to an animal or human subject, for example, of a pharmaceutically effective amount of the compound of the present disclosure, when the compound is formulated together with a pharmaceutically active carrier, diluent or excipient, as hereinbefore described, to thereby treat the subject. Upon having contacted the 5-HT_{2A} receptor, the compound may activate the 5-HT_{2A} receptor or inhibit the 5-HT_{2A} receptor.

Thus, in a further aspect, the condition that may be treated in accordance herewith can be any 5-HT_{2A} receptor mediated disorder. Such disorders include, but are not limited to schizophrenia, psychotic disorder, attention deficit hyperactivity disorder, autism, and bipolar disorder.

The chemical compounds of the present disclosure may also be used as a feedstock material for other psilocybin derivatives. Thus in one embodiment, the chemical compounds of the present disclosure may be in used manufacture of a pharmaceutical or recreational drug formulation, wherein the manufacture may comprise derivatizing a chemical compound having the formula (I):

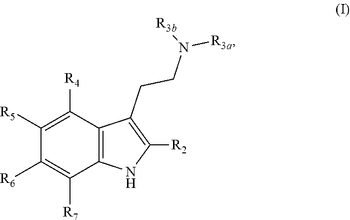

wherein, at least one of R₂, R₄, R₅, R₆ or R₇ is a hydroxy group, wherein each non-hydroxylated R₂, R₅, R₆, or R₇ is a hydrogen atom, wherein R₄ when it is not hydroxylated is a phosphate group, a hydrogen atom or an alkyl group or O-alkyl group, and wherein R_{3A} and R_{3B} are a hydrogen atom, an alkyl group, a cycloalkyl group, an alkaryl group, an aryl group, or an acyl group, or a salt of the chemical compound.

In order to use the compound having formula (I) as a feedstock, one or more hydroxy groups may be replaced by any atoms or groups, for example hydrocarbon groups. Those of skill in the art will be generally familiar with methods that may be used to substitute hydroxy groups. In this respect, guidance may be found in Schnepel C. et al. (2017) Chem. Eur. J. 23:12064-12086; Durak L. J. et al. (2016) ACS Catal. 6:1451; Runguphan W. et al. (2013) Org Lett 15:2850; Corr M. J. et al. (2017) Chem. Sci. 8:2039; and Roy A. D. et al. Chem. Comm. 4831.

Turning now to methods of making the hydroxylated psilocybin derivatives, it is noted that the psilocybin compounds of the present disclosure may be prepared in any suitable manner, including any organic chemical synthesis methods, biosynthetic methods, or a combination thereof. Synthesis generally may involve selecting a psilocybin precursor compound, and modifying the psilocybin precursor compound to form a hydroxylated psilocybin derivative. In this respect, it is noted that a non-hydroxylated psilocybin derivative may be selected and modified to form psilocybin, which subsequently may be hydroxylated, or, alternatively, a hydroxylated psilocybin derivative may be selected to subsequently form a hydroxylated psilocybin derivative. Suitable psilocybin precursor compounds include compounds comprising an indole prototype structure (see: FIG. 2), including, for example tryptophan, tryptamine, 4-hydroxyindole, 4-hydroxytryptophan, 4-hydroxytryptamine, norbaeocystin and baeocystin, and hydroxylated forms thereof, notably, with respect to the indole prototype structure, 2-, 4-, 5-, 6-, 7-hydroxylated forms thereof. The hydroxy-derivative psilocybin precursor compounds may be provided in a more or less chemically pure form, for example, in the form of a psilocybin precursor preparation having a purity of at least about 95%, at least about 96%, at least about 97%, at least about 98%, at least about 99%, or at least 99.9%. The psilocybin precursor compounds may be chemically synthesized, or obtained from a fine chemical manufacturer.

In one embodiment of the present disclosure the hydroxylated psilocybin derivatives may be formed biosynthetically. Accordingly, the present disclosure further includes in one embodiment, a method of making a hydroxylated psilocybin derivative the method comprising:
(a) contacting a psilocybin precursor compound with a host cell comprising a psilocybin biosynthetic enzyme complement; and
(b) growing the host cell to produce a hydroxylated psilocybin derivative compound having the formula (I):

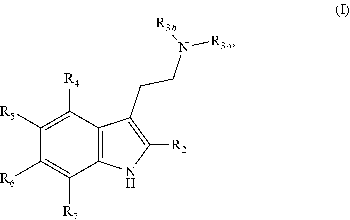

wherein, at least one of R₂, R₄, R₅, R₆, or R₇ is a hydroxy group, and wherein each non-hydroxylated R₂, R₅, R₆, or R₇ is a hydrogen atom or an alkyl group or O-alkyl group, wherein R₄ when it is not hydroxylated is a phosphate group, a hydrogen atom or an alkyl group or O-alkyl group, and wherein R_{3A} and R_{3B} are a hydrogen atom, an alkyl group, a cycloalkyl group, an alkaryl group, an aryl group, or an acyl group.

Implementation of the foregoing example embodiment initially involves providing psilocybin precursor compounds and host cells having a psilocybin biosynthetic enzyme complement. Accordingly, next, exemplary psilocybin precursor compounds and example host cells that may be selected and used in accordance with the present disclosure will be described. Thereafter, example methodologies and techniques will be described to contact and use the psilocybin precursor compounds and cells to produce example hydroxylated psilocybin compounds.

In some embodiments, hydroxy-derivative psilocybin precursor compounds may be selected, prepared and used, i.e. psilocybin precursor compounds possessing or derivatized to possess one or more hydroxy groups, including a hydroxylated tryptophan, e.g. C₂, C₄, C₅, C₆, and/or C₇ hydroxylated tryptophan, a hydroxylated indole, e.g. C₂, C₄, C₅, C₆, and/or C₇ hydroxylated indole, or a hydroxylated tryptamine e.g. C₂, C₄, C₅, C₆, and/or C₇ hydroxylated tryptamine, and further includes alkyl or O-alkyl derivatives thereof e.g. C₂, C₄, C₅, C₆, and/or C₇ methyl, ethyl, propyl, O-methyl, O-ethyl, O-propyl hydroxylated tryptophan derivatives, hydroxylated indole derivatives and hydroxylated tryptamine derivatives. Hydroxy-derivative psilocybin precursor compounds that may be used in accordance herewith further include 2-hydroxy-indole, 2-hydroxy-tryptophan, 2-hydroxy-tryptamine, 4-hydroxy-indole, 4-hydroxy-tryptophan, 4-hydroxy-tryptamine, 5-hydroxy-indole, 5-hydroxy-tryptophan, 5-hydroxy-tryptamine, 6-hydroxy-indole, 6-hydroxy-tryptophan, 6-hydroxy-tryptamine, 7-hydroxy-indole, 7-hydroxy-tryptophan, 7-hydroxy-tryptamine, 2-hydroxy-5-methyl-indole, 4-hydroxy-5-methyl-indole, 6-hydroxy-5-methyl-indole and 4-hydroxy-7-methyl-indole, 2-hydroxy-5-O-methyl-indole, 4-hydroxy-5-O-methyl-indole, 6-hydroxy-5-O-methyl-indole and 4-hydroxy-7-O-methyl-indole, 2-hydroxy-5-ethyl-indole, 4-hydroxy-5-ethyl-indole, 6-hydroxy-5-ethyl-indole and 4-hydroxy-7-ethyl-indole, 2-hydroxy-5-O-ethyl-indole, 4-hydroxy-5-O-ethyl-indole, 6-hydroxy-5-O-ethyl-indole and 4-hydroxy-7-O-ethyl-indole.

In some embodiments, non-hydroxy-derivative psilocybin precursor compounds may be selected, prepared and used, i.e. any one of the psilocybin precursor compounds selected from tryptophan, tryptamine, and indole, and alkyl or O-alkyl derivatives thereof (e.g. C₂, C₄, C₅, C₆, or C₇ methyl, ethyl, propyl, O-methyl, O-ethyl, O-propyl derivatives). Further non-hydroxy-derivative psilocybin precursor compounds include 2-methyl-indole, 4-methyl-indole, 5-methyl-indole, 6-methyl indole, 7-methyl indole, 2-ethyl-indole, 4-ethyl-indole, 5-ethyl-indole, 6-ethyl indole, 7-ethyl indole, 2-methyl-tryptophan, 4-methyl-tryptophan, 5-methyl-tryptophan, 6-methyl tryptophan, 7-methyl tryptophan, 2-ethyl-tryptophan, 4-ethyl-tryptophan, 5-ethyl-tryptophan, 6-ethyl-tryptophan, 7-ethyl-tryptophan, 2-methyl-tryptamine, 4-methyl-tryptamine, 5-methyl-tryptamine, 6-methyl tryptamine, 7-methyl tryptamine, 2-ethyl-tryptamine 4-ethyl-tryptamine, 5-ethyl-tryptamine, 6-ethyl-tryptamine, 7-ethyl-tryptamine

Turning now to the host cells that can be used in accordance with the present disclosure, it is initially noted that a variety of host cells may be selected in accordance with the present disclosure, including microorganism host cells, plant host cells, and animal host cells.

In accordance herewith the host cell includes a psilocybin biosynthetic enzyme complement. Such cells can be obtained in at least two ways. First, in some embodiments, host cells may be selected in which a psilocybin biosynthetic enzyme complement is naturally present. Generally cells naturally producing psilocybin for example, cells of fungal species belonging to the genus Psilocybe, are suitable in this respect. Second, in some embodiments, a host cell that not naturally produces psilocybin may be modulated to produce a psilocybin biosynthetic enzyme complement. Thus, for example, a nucleic acid sequence encoding a psilocybin biosynthetic enzyme complement may be introduced into a host cell, and upon cell growth the host cells can make the psilocybin biosynthetic enzyme complement.

Typically a nucleic acid sequence encoding one or more enzymes constituting a psilocybin biosynthetic enzyme complement further includes one or more additional nucleic acid sequences, for example, a nucleic acid sequences controlling expression of the one or more enzymes, and these one or more additional nucleic acid sequences together with the nucleic acid sequence encoding the one or more enzymes can be said to form a chimeric nucleic acid sequence.

A host cell which upon cultivation expresses the chimeric nucleic acid can be selected and used in accordance with the present disclosure. Suitable host cells in this respect include, for example, microbial cells, such as bacterial cells, yeast cells, for example, and algal cells or plant cells. A variety of techniques and methodologies to manipulate host cells to introduce nucleic acid sequences in cells and attain expression exists and are well known to the skilled artisan. These methods include, for example, cation based methods, for example, lithium ion or calcium ion based methods, electroporation, biolistics, and glass beads based methods. As will be known to those of skill in the art, depending on the host cell selected, the methodology to introduce nucleic acid material in the host cell may vary, and, furthermore, methodologies may be optimized for uptake of nucleic acid material by the host cell, for example, by comparing uptake of nucleic acid material using different conditions. Detailed guidance can be found, for example, in Sambrook et al., Molecular Cloning, a Laboratory Manual, Cold Spring Harbor Laboratory Press, 2012, Fourth Ed. It is noted that the chimeric nucleic acid is a non-naturally occurring chimeric nucleic acid sequence and can be said to be heterologous to the host cell.

In some embodiments, the one or more enzymes constituting a psilocybin enzyme complement can be selected from by a nucleic acid sequence selected from the nucleic acid sequences consisting of:
(a) SEQ.ID NO: 1, SEQ.ID NO: 3, SEQ. ID NO: 5, SEQ. ID NO: 7, SEQ.ID NO: 9, SEQ.ID NO 11, SEQ.ID NO: 13, SEQ.ID NO: 15, SEQ. ID NO: 17, SEQ.ID NO: 19, SEQ. ID NO: 24, SEQ. ID NO: 26, SEQ. ID NO: 31, SEQ. ID NO: 38 and SEQ. ID NO: 42;
(b) a nucleic acid sequence that is substantially identical to any one of the nucleic acid sequences of (a);
(c) a nucleic acid sequence that is substantially identical to any one of the nucleic acid sequences of (a) but for the degeneration of the genetic code;
(d) a nucleic acid sequence that is complementary to any one of the nucleic acid sequences of (a);
(e) a nucleic acid sequence encoding a polypeptide having any one of the amino acid sequences set forth in SEQ.ID NO: 2, SEQ.ID NO: 4, SEQ.ID NO: 6, SEQ.ID NO: 8, SEQ. ID NO: 10, SEQ.ID NO 12, SEQ. ID NO: 14, SEQ.ID NO: 16, SEQ.ID NO: 18, SEQ.ID NO: 20, SEQ.ID NO: 25, SEQ.ID NO: 27, SEQ. ID NO: 32, SEQ. ID NO: 39 and SEQ.ID NO: 43;
(f) a nucleic acid sequence that encodes a functional variant of any one of the amino acid sequences set forth in SEQ.ID NO: 2, SEQ.ID NO: 4, SEQ.ID NO: 6, SEQ.ID NO: 8, SEQ. ID NO: 10, SEQ.ID NO 12, SEQ. ID NO: 14, SEQ.ID NO: 16, SEQ.ID NO: 18, SEQ.ID NO: 20, SEQ.ID NO: 25, SEQ.ID NO: 27, SEQ. ID NO: 32, SEQ. ID NO: 39 and SEQ.ID NO: 43; and
(g) a nucleic acid sequence that hybridizes under stringent conditions to any one of the nucleic acid sequences set forth in (a), (b), (c), (d), (e) or (f).

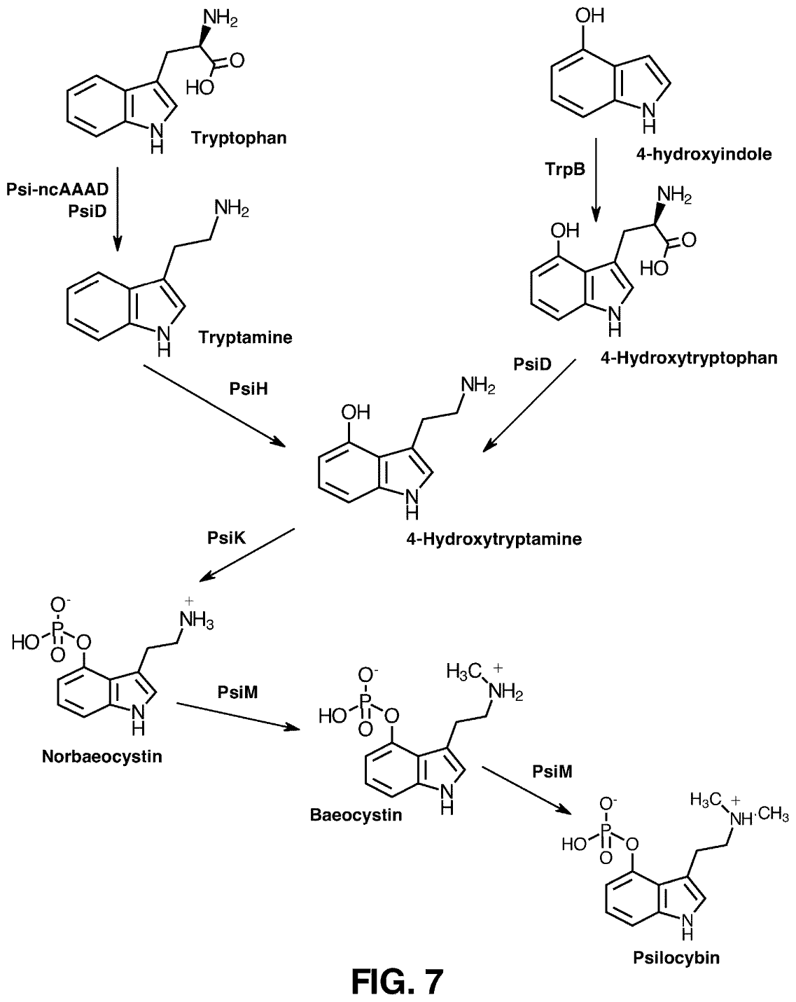

Thus any of the nucleic acid sequences set forth in (a), (b), (c), (d), (e), (f) or (g) may be selected and introduced into a host cell. In general, however the nucleic acid sequence is selected in conjunction with the selected psilocybin precursor compound, as hereinafter further discussed in reference with FIG. 7.

One example host cell that conveniently may be used is Escherichia coli. The preparation of the E. coli vectors may be accomplished using commonly known techniques such as restriction digestion, ligation, gel electrophoresis, DNA sequencing, the polymerase chain reaction (PCR) and other methodologies. A wide variety of cloning vectors is available to perform the necessary steps required to prepare a recombinant expression vector. Among the vectors with a replication system functional in E. coli, are vectors such as pBR322, the pUC series of vectors, the M13 mp series of vectors, pBluescript etc. Suitable promoter sequences for use in E. coli include, for example, the T7 promoter, the T5 promoter, tryptophan (trp) promoter, lactose (lac) promoter, tryptophan/lactose (tac) promoter, lipoprotein (Ipp) promoter, and λ phage PL promoter. Typically, cloning vectors contain a marker, for example, an antibiotic resistance marker, such as ampicillin or kanamycin resistance marker, allowing selection of transformed cells. Nucleic acid sequences may be introduced in these vectors, and the vectors may be introduced in E. coli by preparing competent cells, electroporation or using other well known methodologies to a person of skill in the art. E. coli may be grown in an appropriate medium, such as Luria-Broth medium and harvested. Recombinant expression vectors may readily be recovered from cells upon harvesting and lysing of the cells.

Another example host cell that may be conveniently used is a yeast cell. Example yeast host cells that can be used are yeast cells belonging to the genus Candida, Kluyveromyces, Saccharomyces, Schizosaccharomyces, Pichia, Hansenula, and Yarrowia. In specific example embodiments, the yeast cell can be a Saccharomyces cerevisiae cell, a Yarrowia lipolytica cell, or Pichia pastoris cell.

A number of vectors exist for the expression of recombinant proteins in yeast host cells. Examples of vectors that may be used in yeast host cells include, for example, Yip type vectors, YEp type vectors, YRp type vectors, YCp type vectors, pGPD-2, pAO815, pGAPZ, pGAPZα, pHIL-D2, PHIL-S1, pPIC3.5K, pPIC9K, pPICZ, pPICZα, pPIC3K, pHWO10, pPUZZLE and 2 μm plasmids. Such vectors are known to the art and are, for example, described in Cregg et al., Mol Biotechnol. (2000) 16 (1): 23-52. Suitable promoter sequences for use in yeast host cells are also known and described, for example, in Mattanovich et al., Methods Mol. Biol., 2012, 824:329-58, and in Romanos et al., 1992, Yeast 8:423-488. Examples of suitable promoters for use in yeast host cells include promoters of glycolytic enzymes, like triosephosphate isomerase (TPI), phosphoglycerate kinase (PGK), glyceraldehyde-3-phosphate dehydrogenase (GAPDH or GAP) and variants thereof, lactase (LAC) and galactosidase (GAL), P. pastoris glucose-6-phosphate isomerase promoter (PPGI), the 3-phosphoglycerate kinase promoter (PPGK), the glycerol aldehyde phosphate dehydrogenase promoter (PGAP), translation elongation factor promoter (PTEF), S. cerevisiae enolase (ENO-1), S. cerevisiae galactokinase (GALI), S. cerevisiae alcohol dehydrogenase/glyceraldehyde-3-phosphate dehydrogenase (ADH1 ADH2/GAP), S. cerevisiae triose phosphate isomerase (TPI), S. cerevisiae metallothionein (CUP1), and S. cerevisiae 3-phosphoglycerate kinase (PGK), and the maltase gene promoter (MAL). Marker genes suitable for use in yeast host cells are also known to the art. Thus, antibiotic resistance markers, such as ampicillin resistance markers, can be used in yeast, as well as marker genes providing genetic functions for essential nutrients, for example, leucine (LEU2), tryptophan (TRP1 and TRP2), uracil (URA3, URA5, URA6), histidine (HIS3), and the like. Methods for introducing vectors into yeast host cells can, for example, be found in S. Kawai et al., 2010, Bioeng. Bugs 1(6): 395-403.

Further, guidance with respect to the preparation of expression vectors and introduction thereof into host cells, including in E. coli cells, yeast cells, and other host cells, may be found in, for example: Sambrook et al., Molecular Cloning, a Laboratory Manual, Cold Spring Harbor Laboratory Press, 2012, Fourth Ed.

Thus, to briefly recap, a host cell comprising a chimeric nucleic acid comprising (i) a nucleic acid sequence controlling expression in a host cell and (ii) a nucleic acid sequence encoding a psilocybin biosynthetic enzyme complement, can be prepared in accordance with the present disclosure.

In accordance herewith, host cells are grown to multiply and to express a chimeric nucleic acid. Expression of the chimeric nucleic acid results in the biosynthetic production in the host cell of a psilocybin biosynthetic enzyme complement. Growth media and growth conditions can vary depending on the host cell that is selected, as will be readily appreciated to those of ordinary skill in the art. Growth media typically contain a carbon source, one or several nitrogen sources, essential salts including salts of potassium, sodium, magnesium, phosphate and sulphate, trace metals, water soluble vitamins, and process aids including but not limited to antifoam agents, protease inhibitors, stabilizers, ligands and inducers. Example carbon sources are e.g. mono- or disaccharides. Example nitrogen sources are, e.g. ammonia, urea, amino acids, yeast extract, corn steep liquor and fully or partially hydrolyzed proteins. Example trace metals are e.g. Fe, Zn, Mn, Cu, Mo and H₃BO₃. Example water soluble vitamins are e.g. biotin, pantothenate, niacin, thiamine, p-aminobenzoic acid, choline, pyridoxine, folic acid, riboflavin and ascorbic acid. Further, specific example media include liquid culture media for the growth of yeast cells and bacterial cells including, Luria-Bertani (LB) broth for bacterial cell cultivation, and yeast extract peptone dextrose (YEPD or YPD), for yeast cell cultivation. Further media and growth conditions can be found in Sambrook et al., Molecular Cloning, a Laboratory Manual, Cold Spring Harbor Laboratory Press, 2012, Fourth Ed.

In order for the host cells to produce the hydroxylated psilocybin or psilocin compounds, the cells are provided with a psilocybin precursor compound. Thus, in accordance herewith, host cells may be contacted with a psilocybin precursor compound. In some embodiments, a psilocybin precursor compound can be exogenously supplied, for example, by including a psilocybin precursor compound in the growth medium of the host cells, and growing the host cells in a medium including the psilocybin precursor compound.

Referring next to FIG. 7, shown therein is an example natural biosynthetic pathway showing the conversion of example psilocybin precursor compounds to form psilocybin. Thus, as can be appreciated from FIG. 7, various psilocybin precursor compounds may be selected and prepared in hydroxylated and non-hydroxylated form, in conjunction with a psilocybin biosynthetic enzyme complement.

Thus, referring further to FIG. 7, by way of example, 4-hydroxy-5-methyl indole may be selected as hydroxy-derivative psilocybin precursor compound and contacted with a host cell comprising a psilocybin biosynthetic enzyme complement comprising (i) a nucleic acid sequence encoding TrpB selected from SEQ.ID NO: 11 and SEQ.ID NO: 26 or a sequence substantially identical thereto; (ii) a nucleic acid sequence encoding PsiD selected from SEQ. ID NO: 1, SEQ.ID NO: 9 and SEQ.ID NO: 31 or a sequence substantially identical thereto; and (iii) a nucleic acid sequence encoding an acetyl transferase having SEQ.ID NO: 24 or a sequence substantially identical thereto, and upon growth of the cells 4-hydroxy-5-methyl-tryptamine can be formed, which can further be acetylated by the acetyl transferase (not shown in FIG. 7) to form the hydroxylated psilocybin derivative compound having chemical formula (III):

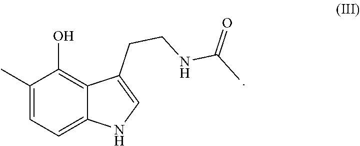 [SMILES: CC(=O)NCCc1c[nH]c2ccc(C)c(O)c12]

Thus, referring further to FIG. 7, by way of a further example, 4-hydroxy-7-methyl indole may be selected as hydroxy-derivative psilocybin precursor compound and contacted with a host cell comprising a psilocybin biosynthetic enzyme complement comprising (i) a nucleic acid sequence encoding TrpB selected from SEQ.ID NO: 11 and SEQ.ID NO: 26 or a sequence substantially identical thereto; (ii) a nucleic acid sequence encoding PsiD selected from SEQ. ID NO: 1, SEQ. ID NO: 9 and SEQ. ID NO: 31 or a sequence substantially identical thereto; and (iii) a nucleic acid sequence encoding an acetyl transferase having SEQ.ID NO: 24 or a sequence substantially identical thereto and upon growth of the cells 4-hydroxy-7-methyl-tryptamine can be formed, which can further be acetylated by the acetyl transferase (not shown in FIG. 7) to form the hydroxylated psilocybin derivative compound having chemical formula (IV):

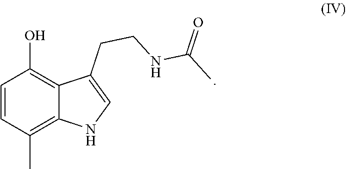 [SMILES: CC(=O)NCCc1c[nH]c2c(C)ccc(O)c12]

It is noted that in some embodiments, the host cells may include a hydroxylase. In such embodiments, the cell may be contacted with a non-hydroxy-derivative psilocybin precursor compound, and hydroxylation may occur in vivo in the host cells. Thus, referring again to FIG. 7, by way of example, tryptophan, indole or tryptamine may be selected and contacted with a host cell including a hydroxylase, and upon growth of the host cells a hydroxylated psilocybin derivative compound can be formed.

Accordingly, in one embodiment the hydroxylase can be encoded by a nucleic acid selected from:
(a) SEQ.ID NO: 13, SEQ.ID NO: 15, SEQ. ID NO: 17, SEQ.ID NO: 19, SEQ.ID NO: 38 or SEQ. ID NO: 42;
(b) a nucleic acid sequence that is substantially identical to any one of the nucleic acid sequences of (a);
(c) a nucleic acid sequence that is substantially identical to any one of the nucleic acid sequences of (a) but for the degeneration of the genetic code;
(d) a nucleic acid sequence that is complementary to any one of the nucleic acid sequences of (a);
(e) a nucleic acid sequence encoding a polypeptide having any one of the amino acid sequences set forth in SEQ.ID NO: 14, SEQ.ID NO: 16, SEQ.ID NO: 18, SEQ. ID NO: 20, SEQ. ID NO: 39 and SEQ.ID NO: 43;
(f) a nucleic acid sequence that encodes a functional variant of any one of the amino acid sequences set forth in SEQ.ID NO: 14, SEQ.ID NO: 16, SEQ.ID NO: 18, SEQ.ID NO: 20, SEQ. ID NO: 39 and SEQ. ID NO: 43; and
(g) a nucleic acid sequence that hybridizes under stringent conditions to any one of the nucleic acid sequences set forth in (a), (b), (c), (d), (e) or (f).

Thus, referring further to FIG. 7, by way of example, 7-ethyl-indole may be selected as a non-hydroxy-derivative psilocybin precursor compound and contacted with a host cell comprising a psilocybin biosynthetic enzyme complement comprising (i) a nucleic acid sequence encoding TrpB selected from SEQ.ID NO: 11 and SEQ. ID NO: 26 or a sequence substantially identical thereto; (ii) a nucleic acid sequence encoding PsiD selected from SEQ.ID NO: 1, SEQ.ID NO: 9 and SEQ. ID NO: 31 or a sequence substantially identical thereto; and (iii) a nucleic acid sequence encoding a hydroxylase selected from SEQ.ID NO: 14, SEQ.ID NO: 16, SEQ.ID NO: 18, SEQ.ID NO: 20, SEQ. ID NO: 39 and SEQ.ID NO: 43 or a sequence substantially identical thereto, and upon growth of the cells the hydroxylated psilocybin derivative compound having chemical formula (XXXII):

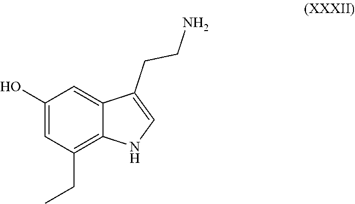

can be formed.

It will be clear to those of skill in the art that a significant variety of different hydroxy- and non-hydroxy-derivative psilocybin precursor compounds may be selected. FIG. 7 in this respect provides guidance and allows a person of skill in the art to select appropriate psilocybin precursor compounds and a matching a psilocybin biosynthetic enzyme complement.

Upon production by the host cells of the hydroxylated psilocybin compounds in accordance with the methods of the present disclosure, the hydroxylated psilocybin compounds may be extracted from the host cell suspension, and separated from other constituents within the host cell suspension, such as media constituents and cellular debris. Separation techniques will be known to those of skill in the art and include, for example, solvent extraction (e.g. butane, chloroform, ethanol), column chromatography based techniques, high-performance liquid chromatography (HPLC), for example, and/or countercurrent separation (CCS) based systems. The recovered hydroxylated psilocybin compounds may be obtained in a more or less pure form, for example, a preparation of hydroxylated psilocybin compounds of at least about 60% (w/v), about 70% (w/v), about 80% (w/v), about 90% (w/v), about 95% (w/v) or about 99% (w/v) purity may be obtained. Thus, in this manner, hydroxylated psilocybin derivatives in more or less pure form may be prepared.

In further embodiments, in accordance herewith hydroxylated psilocybin compounds may be chemically synthesized, using, for example, 4-benzyloxyindole as a starting compound. Example techniques for chemically synthesizing the hydroxylated psilocybin compounds of the present disclosure are further hereinafter described in Examples 4 to 14.

It will now be clear form the foregoing that novel hydroxylated psilocybin derivatives are disclosed herein. The hydroxylated psilocybin compounds may be formulated for use as a pharmaceutical drug or recreational drug. The hydroxylated psilocybin compounds may also be used as a feedstock to produce other psilocybin derivatives.

### SUMMARY OF SEQUENCES

SEQ.ID NO: 1 sets forth a Psilocybe cubensis nucleic acid sequence encoding a PsiD polypeptide.

SEQ.ID NO: 2 sets forth a deduced amino acid sequence of a Psilocybe cubensis PsiD polypeptide.

SEQ.ID NO: 3 sets forth a Psilocybe cubensis nucleic acid sequence encoding a PsiH polypeptide.

SEQ.ID NO: 4 sets forth a deduced amino acid sequence of a Psilocybe cubensis PsiH polypeptide.

SEQ.ID NO: 5 sets forth a Psilocybe cubensis nucleic acid sequence encoding a PsiK polypeptide.

SEQ.ID NO: 6 sets forth a deduced amino acid sequence of a Psilocybe cubensis PsiK polypeptide.

SEQ.ID NO: 7 sets forth a Psilocybe cubensis nucleic acid sequence encoding a PsiM polypeptide.

SEQ.ID NO: 8 sets forth a deduced amino acid sequence of a Psilocybe cubensis PsiM polypeptide.

SEQ.ID NO: 9 sets forth a Psilocybe cubensis nucleic acid sequence encoding a Psi-ncAAAD polypeptide.

SEQ.ID NO: 10 sets forth a deduced amino acid sequence of a Psilocybe cubensis Psi-ncAAAD polypeptide.

SEQ.ID NO: 11 sets forth a Psilocybe cubensis nucleic acid sequence encoding a TrpB polypeptide.

SEQ.ID NO: 12 sets forth a deduced amino acid sequence of a Psilocybe cubensis TrpB polypeptide.

SEQ.ID NO: 13 sets forth an Oryza sativa nucleic acid sequence encoding tryptamine 5-hydroxylase polypeptide.

SEQ.ID NO: 14 sets forth a deduced amino acid sequence of an Oryza sativa tryptamine 5-hydroxylase polypeptide.

SEQ.ID NO: 15 sets forth a Rattus norvegicus nucleic acid sequence encoding tryptophan 5-hydroxylase polypeptide.

SEQ.ID NO: 16 sets forth a deduced amino acid sequence of a Rattus norvegicus tryptophan 5-hydroxylase polypeptide.

SEQ.ID NO: 17 sets forth an Oryza sativa nucleic acid sequence encoding melatonin 2-hydroxylase polypeptide.

SEQ.ID NO: 18 sets forth a deduced amino acid sequence of an Oryza sativa melatonin 2-hydroxylase polypeptide.

SEQ.ID NO: 19 sets forth an Aspergillus nidulans nucleic acid sequence encoding tryptamine N-acetyltryptophan 6-hydroxylase polypeptide.

SEQ.ID NO: 20 sets forth a deduced amino acid sequence of an Aspergillus nidulans N-acetyltryptophan 6-hydroxylase polypeptide.

SEQ.ID NO: 21 sets forth a nucleic acid sequence of pCDM4 vector.

SEQ.ID NO: 22 sets forth a nucleic acid sequence encoding a synthetic FLAG epitope tag polypeptide

SEQ.ID NO: 23 sets forth a deduced amino acid sequence of a synthetic FLAG epitope tag polypeptide

SEQ.ID NO: 24 sets forth a nucleic acid sequence encoding a Streptomyces griseofuscus PsmF N-acetyltransferase polypeptide.

SEQ.ID NO: 25 sets forth a deduced amino acid sequence of a Streptomyces griseofuscus PsmF N-acetyltransferase polypeptide.

SEQ.ID NO: 26 sets forth a nucleic acid sequence encoding a mutated Thermotoga maritima TmTrpB-2F3 tryptophan synthase subunit B polypeptide.

SEQ.ID NO: 27 sets forth a deduced amino acid sequence of a mutated Thermotoga maritima TmTrpB-2F3 tryptophan synthase subunit B polypeptide.

SEQ.ID NO: 28 sets forth a nucleic acid sequence encoding a synthetic V5 epitope tag polypeptide

SEQ.ID NO: 29 sets forth a deduced amino acid sequence of a synthetic V5 epitope tag polypeptide

SEQ.ID NO: 30 sets forth a nucleic acid sequence of pETM6-H10 vector

SEQ.ID NO: 31 sets forth a nucleic acid sequence encoding a Bacillus atrophaeus BaTDC tryptophan decarboxylase polypeptide.

SEQ.ID NO: 32 sets forth a deduced amino acid sequence of a Bacillus atrophaeus BaTDC tryptophan decarboxylase polypeptide.

SEQ.ID NO: 33 sets forth a nucleic acid sequence encoding a synthetic HIS epitope tag polypeptide

SEQ.ID NO: 34 sets forth a deduced amino acid sequence of a synthetic HIS epitope tag polypeptide

SEQ.ID NO: 35 sets forth a nucleic acid sequence of pET28a(+) vector

SEQ.ID NO: 36 sets forth a nucleic acid sequence encoding a synthetic c-MYC epitope tag polypeptide

SEQ.ID NO: 37 sets forth a deduced amino acid sequence of a synthetic c-MYC epitope tag polypeptide

SEQ.ID NO: 38 sets forth a nucleic acid sequence encoding an Oryza sativa OsCPR2 cytochrome P450 reductase-2 polypeptide,

SEQ.ID NO: 39 sets forth a deduced amino acid sequence of an Oryza sativa OsCPR2 cytochrome P450 reductase-2 polypeptide.

SEQ.ID NO: 40 sets forth a nucleic acid sequence encoding a synthetic HA epitope tag polypeptide

SEQ.ID NO: 41 sets forth a deduced amino acid sequence of a synthetic HA epitope tag polypeptide

SEQ.ID NO: 42 sets forth a nucleic acid sequence encoding an N-terminal GST tagged Oryza sativa OsCYP71p cytochrome P450 polypeptide.

SEQ.ID NO: 43 sets forth deduced amino acid sequence of an N-terminal GST tagged Oryza sativa OsCYP71p cytochrome P450 polypeptide.

SEQ.ID NO: 44 sets forth a nucleic acid sequence of pCDFDuet-1 vector

Hereinafter are provided examples of specific implementations for performing the methods of the present disclosure, as well as implementations representing the compositions of the present disclosure. The examples are provided for illustrative purposes only, and are not intended to limit the scope of the present disclosure in any way.

### EXAMPLES

### Example 1—Process for Biosynthetically Making a First Hydroxylated Psilocybin Derivative from Hydroxylated Indole Feedstock

E. coli strain Ec-1 was constructed as follows. For plasmid cloning, Top10 or XL1-blue strains were used depending on antibiotic markers. Standard LB media was used for culturing. For gene expression and feeding experiments, the parent host strain employed was BL21 (DE3). From plasmid pCDM4 (SEQ.ID NO: 21), the plasmid pCDM4-PsmF-FLAG was created by inserting an in-frame, C-terminally FLAG-tagged (SEQ.ID NO: 22; SEQ.ID NO: 23) PsmF gene (SEQ.ID NO: 24) into the NdeI/XhoI site of pCDM4. The plasmid pETM6-H10-TmTrpB-2F3-V5-BaTDC-FLAG was created by first cloning the in-frame, C-terminally V5-tagged (SEQ.ID NO: 28) TmTrpB-2F3 (SEQ.ID NO: 26) into the NdeI/XhoI site of pETM6-H10 (SEQ.ID NO: 30) to create pETM6-H10-TmTrpB-2F3-V5. This intermediate plasmid was digested with SpeI and SaII, and in-frame, C-terminally FLAG tagged (SEQ.ID NO: 22) BaTDC (SEQ.ID NO: 31) was cloned into the site with XbaI and SaII, nullifying the SpeI restriction site. In this setup, the T7 polymerase was able to drive the expression of the polycistronic DNA containing both TmTrpB-2F3 and BaTDC. The two target plasmids pCDM4-PsmF-FLAG and pETM6-H10-TmTrpB-2F3-V5-BaTDC-FLAG were transformed into BL21 (DE3) cells, and antibiotics ampicillin plus streptomycin were used to select for the correct clones containing both plasmids. Scaled-up culturing of engineered E. coli was conducted as follows: seed cultures were inoculated in AMM (Jones et al. 2015, Sci Rep. 5:11301) medium overnight. The overnight culture was then divided into two flasks containing 500 mL each of AMM medium additionally containing 0.5% (w/v) serine, 1M IPTG, 50 ug/L ampicillin and streptomyces, and 100 mg/L 4-hydroxy-7-methylindole (BLDPharm, www.bldpharm.com) for conversion by Ec-1. Cultures were grown for 24 h. Cultures were then centrifuged (10,000g×5 minutes) to remove cellular content, and culture broth containing secreted derivative was stored at-80C until further processing. The N-[2-(4-hydroxy-7-methyl-1H-indol-3-yl)ethyl]acetamide product having chemical formula (IV):

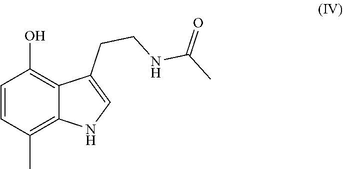 [SMILES: CC(=O)NCCc1c[nH]c2c(C)ccc(O)c12]

contained in 1 L of E. coli culture was extracted by ethyl acetate (3×600 ml). The organic layer was dried over Na₂SO₄, followed by concentration under reduced pressure. The residue was purified by flash chromatography on silica gel using ethyl acetate-hexane (50→70%) as eluent, followed by crystallization from acetone/hexane to give the compound as a pink solid (80 mg). Following purification, high-resolution MS (HRMS), ¹H NMR, and selective ¹³C NMR were performed to assess purity, estimate total quantity, and confirm molecular structure. ¹H NMR (400 MHZ, CD₃OD): δ=1.89 (s, 3H), 2.38 (s, 3H), 3.14 (t, J=6.7 Hz, 2H), 3.49 (t, J=6.9 Hz, 2H), 6.28 (d, J=7.6 Hz, 1H), 6.68 (dd, J=7.6, 0.6 Hz, 1H), 6.91 (s, 1H). ¹³C NMR (100 MHZ, CD₃OD): δ=14.8, 21.2, 25.9, 41.7, 102.7, 111.9, 112.7, 116.3, 120.9, 121.8, 138.1, 149.4, 171.8. HRMS (ESI) m/z: calcd. for C₁₃H₁₆N₂O₂ [M+H]⁺ 233.1284, found 233.1283. The purity of compound (IV) was determined to be 95% (w/w).

Cell lines for pharmacology assays. CHO-K1/Galpha15 (GenScript, M00257) (-5-HT_{2A}) and CHO-K1/5-HT_{2A} (GenScript, M00250) (+5-HT_{2A}) cells lines were used in both toxicology/growth inhibition (MTT) and calcium release assays. Briefly, CHO-K1/Galpha15 is a control cell line that constitutively expresses Galpha15 which is a promiscuous Gq protein. It is engineered as a host cell, allowing transfected receptor(s) to signal through the Gq signal transduction pathway and mobilize intracellular calcium from the endoplasmic reticulum (ER). These control cells lack any transgene encoding 5-HT_{2A} receptors, thus preventing calcium mobilization in response to 5-HT_{2A} activation. Conversely, CHO-K1/5-HT_{2A} cells stably express 5-HT_{2A} receptor in the CHO-K1 host background. This design enables Gq-11 expressed in CHO-K1 cells to mobilize intracellular calcium changes when 5-HT_{2A} receptors are activated by ligands.

Cell lines were maintained in Ham's F12 media plus 10% FBS in the presence of 100 ug/ml hygromycin for CHO-K1/Ga15 or 400 μg/ml G418 for CHO-K1/5-HT_{2A} unless indicated otherwise for specific assays. Cell maintenance was carried out as recommended by the cell supplier. Briefly, vials with cells were removed from the liquid nitrogen and thawed quickly in 37° C. water bath. Just before cells were completely thawed, vial exteriors were decontaminated with 70% ethanol spray. Cell suspension was then retrieved from the vial and added to warm (37° C.), ‘complete’ (non-dropout) growth media, and centrifuged at 1,000 rpm for 5 minutes. The supernatant was discarded and the cell pellet was then resuspended in another 10 ml of complete growth media, and added to a 10 cm cell culture dish (Greiner Bio-One #664160). The media was changed every third day until the cells reached ˜90% confluence. The ˜90% confluent cells were then split 10:1, and used either for maintenance or pharmacological study.

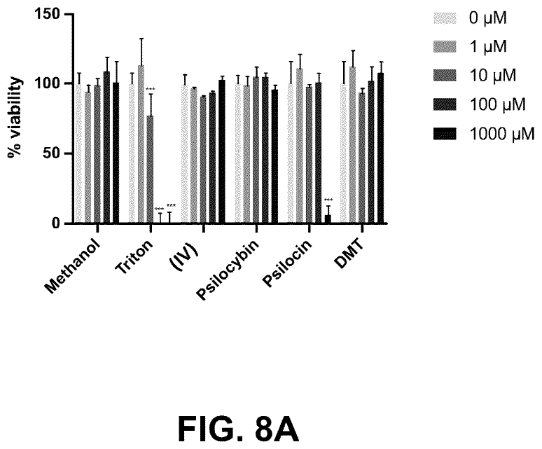

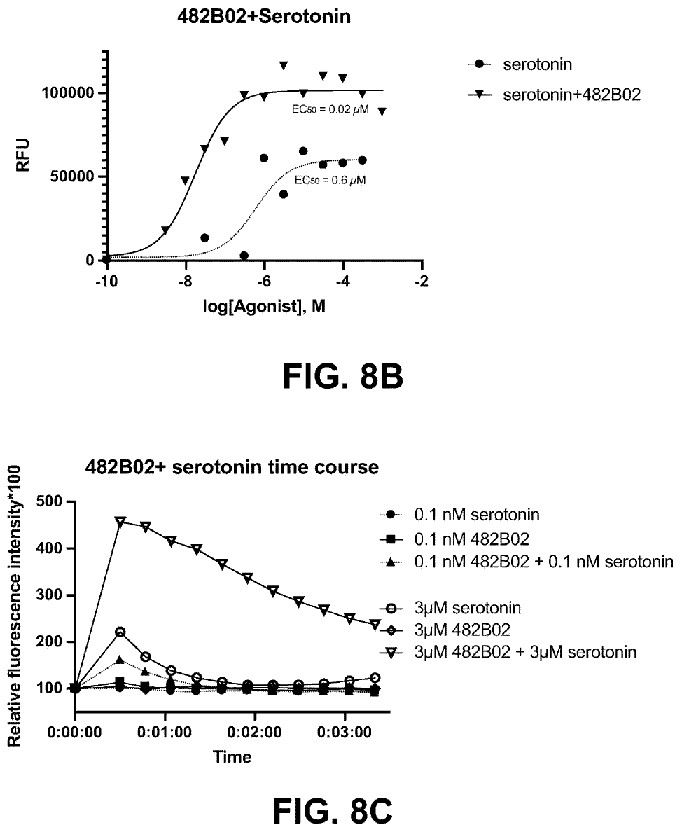

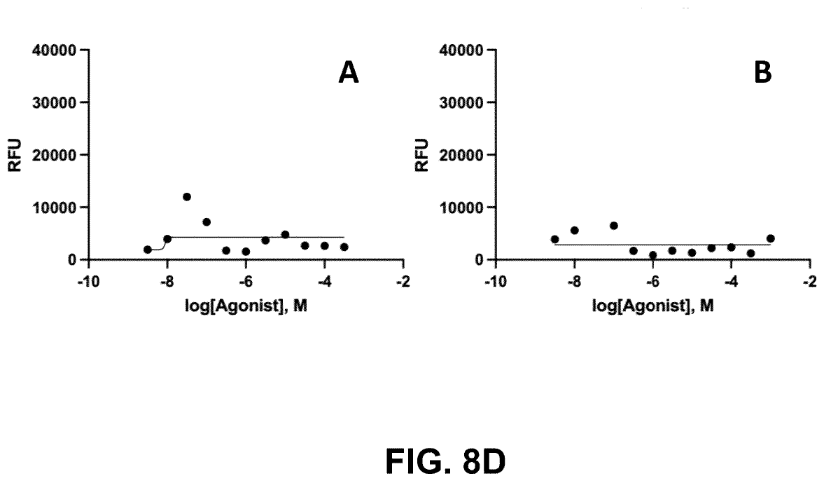

Assessment of cell viability upon treatment of hydroxylated psilocybin derivatives. To establish suitable ligand concentrations for the calcium release assays, MTT (3-(4,5-dimethylthiazol-2-yl)-2,5-diphenyltetrazolium) assays were first performed. Results of these assays were conducted using both control ligands (e.g. psilocybin, psilocin, DMT) and novel derivatives, in part as a pre-screen for any remarkable toxic effects on cell cultures up to concentrations of 1 mM. A known cellular toxin (Triton X-100, Pyrgiotakis G. et al., 2009, Ann. Biomed. Eng. 37:1464-1473) was included as a general marker of toxicity. Modified Chinese Hamster Ovary cells (CHO-K1/Ga15) were cultured using standard procedures using the manufacture's protocols (Genscript, M00257). Briefly, cells were cultured in Ham's F12 medium supplemented with 10% fetal bovine serum and 100 mg/ml hygromycin B, and grown at 37° C. in the presence of 5% CO₂. To test the various compounds with the cell line, cells were seeded in a clear 96-well culture plate at 10,000 cells per well. After allowing cells to attach and grow for 24 hours, assay compounds were added at 1 μM, 10 μM, 100 μM, and 1 mM final concentrations. Methanol concentrations used are 0.001, 0.01, 0.1, and 1%. Triton concentrations used are 0.0001, 0.001, 0.01 and 0.1%. Cells were incubated with compounds for 48 hours before accessing cell viability with the MTT assay following the manufacture's protocol (MTT Cell Growth Assay Kit; Millipore Sigma, CT02). MTT reagent was added to cells and allowed to incubate for 4 hours before solubilization with isopropanol plus 0.04 N HCl. Absorbance readings were performed at 570 nm with the reference at 630 nm on a SpectraMax iD3 plate reader. Non-treated cells were assigned 100% viability. Results of the cell viability assays are shown in FIG. 8A. Bar graphs show the mean+/−SD (n=3). Significance (P<0.0001), as indicated by (***) was determined using 2-way ANOVA with Dunnett's multiple comparisons test. The results using compound with formula (IV) are indicated as “(IV)” on the x-axis.

Increase in cytosolic calcium concentration by 5-HT_{2A} activation and assessment of modulation. Changes in intracellular calcium concentration due to the treatment with assay compounds was measured using Fluo-8 dye (Abcam, #ab112129) according to the manufacturer's instructions. Briefly, CHO-K1 cells stably expressing 5-HT_{2A} (Genscript #M00250) (+5-HT_{2A}) or lacking 5-HT_{2A} (Genscript, M00257) (-5-HT_{2A}) were seeded on black walled clear bottom 96-well plates (Thermo Scientific #NUNC165305), allowing 70,000 cells/well in 100 ul media (HAM's F12, GIBCO #11765-047) with 1% FBS (Thermo Scientific #12483020). Cultures were maintained in a humidified incubator at 37° C. and 5% CO₂. Fluo-8 dye was loaded into the cultures for 30 min at 37° C., followed by 30 min additional incubation at room temperature. Next, different dilutions of novel molecules and controls were prepared in serum-free culture media and added to the cells. Fluorescence (ex 490 nm/em 525 nm) obtained after the addition of molecules was expressed relative to values obtained before addition of the molecules (relative Fluo-8 fluorescence=Fmax/F0, where Fmax=maximum fluorescence and F0=baseline fluorescence). Fluorescence intensities were measured using a Spectramax ID3 plate reader (www.moleculardevices.com). Relative fluorescence (RFU) data was subjected to four parameter logistic curve fittings to determine EC₅₀ for serotonin with the aid of GraphPad Prism (Version 9.2.0). Serotonin (5-hydroxytryptamine, 5-HT) is a known agonist with binding activity to 5HT_{2A} (Gothert, 2013, M. Pharmacol. Rep 65:771-786; Kim K. et al., 2020, Cell 182:1574-1588). Serotonin was assayed with and without compound (IV) in +5-HT_{2A} cells to evaluate potential for positive allosteric modulation (PAM) Fasciani, I. et al., 2020, Pharmaceuticals 13:388; Gao Z. and Jacobsen K. A., 2013, Drug Discov. Today Technol. 10: e237-e243). FIG. 8B shows a shift in serotonin EC₅₀ in the presence of compound (IV), which is referred to in FIG. 8B as “482B02”.

Relative fluorescence (RFU) was then evaluated with respect to time (seconds) illustrating time-dependent calcium flux in response to serotonin with, or without, compound (IV). Results shown in FIG. 8C illustrate elevated flux in response to serotonin (3 uM) with compound (IV) (3 uM) compared to serotonin (3 uM) alone. Compound (IV) is referred to as “482B02” in FIG. 8C.

Conversely, -5-HT_{2A} cells (CHO-K1/Galpha15, GenScript, M00257) did not exhibit a response to serotonin (FIG. 8D (panel A), serotonin is “Agonist”) and application of methanol vehicle did not elicit a response in +5-HT_{2A} cells (FIG. 8D (Panel B), vehicle is “Agonist”).

### Example 2—Process for Biosynthetically Making a Second Hydroxylated Psilocybin Derivative from Hydroxylated Indole Feedstock

Escherichia coli strain Ec-1 was used to biosynthesize hydroxylated tryptamine derivative with formula (III) from hydroxylated indole feedstock. The construction of Ec-1 is described in Example 1. Scaled-up culturing and material storage of engineered E. coli was conducted as described in Example 1, except that 4-hydroxy-5-methylindole (Combi-Blocks, www.combi-blocks.com) was used in place of 4-hydroxy-7-methylindole. N-[2-(4-Hydroxy-5-methyl-1H-indol-3-yl)ethyl]acetamide product having chemical formula (III):

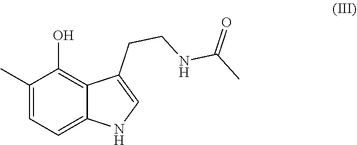 [SMILES: CC(=O)NCCc1c[nH]c2ccc(C)c(O)c12]

contained in 0.5 litre of broth was extracted by ethyl acetate (3×300 ml). The organic layer was dried over Na₂SO₄, followed by concentration under reduced pressure. The residue was purified by flash chromatography on silica gel using ethyl acetate-hexane (50→70%) as eluent, followed by crystallization from acetone/hexane to give the compound as a light yellow solid (8 mg). Following purification, high-resolution MS (HRMS), ¹H NMR, and selective ¹³C NMR were performed to assess purity, estimate total quantity, and confirm molecular structure. ¹H NMR (400 MHZ, CD₃OD): δ=1.90 (s, 3H), 2.28 (s, 3H), 3.08 (t, J=6.5 Hz, 2H), 3.49 (t, J=7.1 Hz, 2H), 6.79 (d, J=0.9 Hz, 2H), 6.86 (s, 1H). ¹³C NMR (100 MHZ, CD₃OD): δ=14.4, 21.1, 26.1, 41.7, 103.3, 111.4, 111.7, 117.7, 121.2, 124.5, 137.9, 147.8, 171.9. HRMS (ESI) m/z: calcd. for C₁₃H₁₆N₂O₂ [M+H]⁺ 233.1284, found 233.1283. The purity of compound (III) was determined to be 95% (w/w).

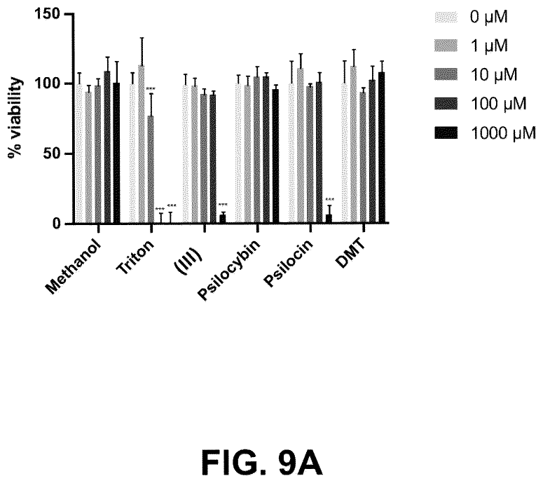

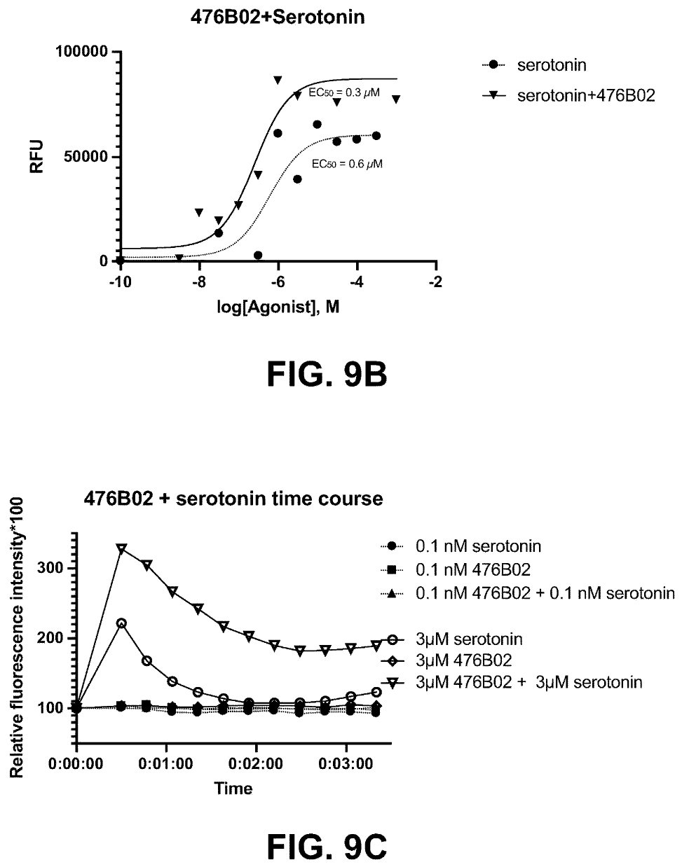

Efficacy testing was carried out as described in Example 1. MTT assay results are shown in FIG. 9A, and calcium mobility assay results are shown in FIGS. 9B and 9C. In the MTT assay, the example compound of the chemical formula (III) is shown as “III”. In the calcium mobility assays, the Example compound of the chemical formula (III) is referred to as “476B02”.

### Example 3—Process for Biosynthetically Making a Third Hydroxylated Psilocybin Derivative from Hydroxylated Indole Feedstock

E. coli strain Ec-2 was constructed as follows. For plasmid cloning, Top10 or XL1-blue strains were used depending on antibiotic markers. Standard LB media was used for culturing. For gene expression and feeding experiments, the parent host strain employed was BL21 (DE3). For this Example, heterologous expression of a non-native or engineered TrpB gene was not necessary, as endogenous tryptophan synthase activity proved sufficient. The plasmid pET28a(+)-BaTDC-HIS was created by inserting the HIS tagged (SEQ.ID NO: 33) BaTDC gene (SEQ.ID NO: 31) into the NdeI/XhoI site of pET28a(+) (SEQ.ID NO: 35). The plasmid pCDFDuet-OsCPR2-Myc-GST-d37OsCYP71p-HA was created by first cloning the c-MYC tagged (SEQ.ID NO: 36) OsCPR2 gene (SEQ.ID NO: 38) into the MCS1 of pCDFDuet (SEQ.ID NO: 44) and then cloning HA tagged (SEQ.ID NO: 40) GST-d37OsCYP71p (SEQ.ID NO: 42) into the MCS2 of the same plasmid. In this setup, two different genes are driven in tandem by two different T7 promoters on the same plasmid. Scaled-up culturing and material storage of engineered E. coli was conducted as described in Example 1, except that 7-ethylindole (Combi-Blocks, www.combi-blocks.com) was used in place of 4-hydroxy-7-methylindole. Analysis was carried out using high-resolution LC-HESI-LTQ-Orbitrap-XL MS (Thermo Fisher Scientific), employing a modified version of a method described previously (Chang et al., 2015, Plant Physiol. 169:1127-1140), with the exception that liquid chromatography was carried out using an UltiMate 3000 HPLC (Thermo Fisher Scientific) equipped with a Poroshell 120 SB-C18 column (Agilent Technologies) instead of an Accela HPLC system (Thermo Fisher Scientific) equipped with a Zorbax C18 column (Agilent Technologies). Briefly, 10 microliters of culture media was injected at a flow rate of 0.5 mL/min and a gradient of solvent A (water with 0.1% of formic Acid) and solvent B (ACN with 0.1% formic Acid) as follows: 100% to 0% (v/v) solvent A over 5 min; isocratic at 0% (v/v) for 1 min; 0% to 100% (v/v) over 0.1 min; and isocratic at 100% (v/v) for 1.9 min. Total run time was 8 minutes. Heated ESI source and interface conditions were operated in positive ion mode as follows: vaporizer temperature, 400° C.; source voltage, 3 kV; sheath gas, 60 au, auxiliary gas, 20 au; capillary temperature, 380° C.; capillary voltage, 6 V; tube lens, 45 V. Instrumentation was performed as a single, HR scan event using Orbitrap detection of m/z in the range of 100-700 m/z. Ion injection time was 300 ms with scan time of 1 s. External and internal calibration procedures ensured <2 ppm error to facilitate elemental formulae predictions. Singly protonated product with exact m/z and expected elemental formula matching protonated 3-(2-aminoethyl)-7-ethyl-1H-indol-5-ol having chemical formula (XXXII):

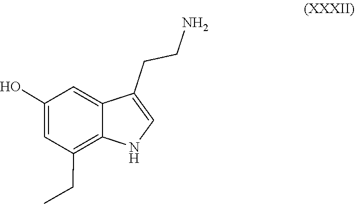

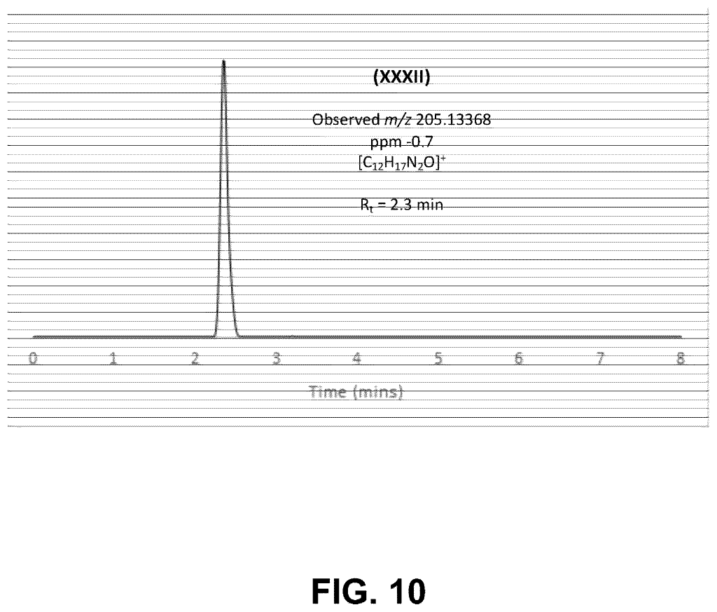

eluted at 2.3 minutes (EIC, see: FIG. 10).

### Example 4—Process for Making a Fourth Hydroxylated Psilocybin Derivative Using Synthetic Chemistry

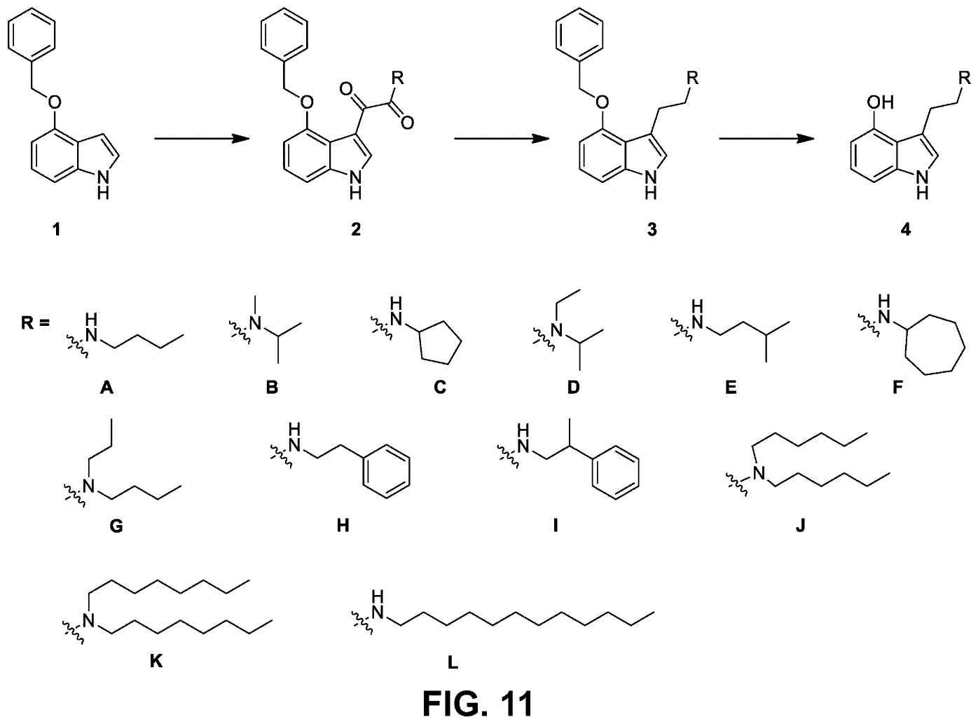

This example describes an example process for chemically synthesizing hydroxylated psilocybins. The general synthetic procedure shown in FIG. 11 was followed.

To a solution of 4-benzyloxyindole 1 (1.00 mmol, 1.00 eq) dissolved in anhydrous diethyl ether (10 mL) under argon sparging at 0° C., was added oxalyl chloride (2.05 mmol, 2.05 eq) dropwise over the course of 30 minutes, and the reaction was continued at 0-5° C. for 3 hours. A solution of N-ethyl(isopropyl)amine (9.00 total eq, required for R group “D” in FIG. 11) in anhydrous diethyl ether (5 mL) was added dropwise over the course of 1 hour. The diethyl ether was removed in vacuo, and the residue was redissolved in dichloromethane (30 mL). The organic solution was washed with water (4×10 mL) and brine (10 mL), then dried with MgSO₄ and concentrated under vacuo to yield 2, which was used without purification in the following step. A solution of lithium aluminum hydride in anhydrous THF (1 M, 5.20 eq) in dry 1,4-dioxane (2.0 mL) was brought to 60° C. under argon. A solution of 2 in a mixture of anhydrous THF (2.0 mL) and 1,4-dioxane (3.5 mL) was added dropwise over 30 minutes, and the reaction mixture was brought to 70° C. for 2 hours, then to reflux at 95° C. for 20 hours. After cooling to 0° C., excess lithium aluminum hydride was quenched through the dropwise addition of a mixture of water (0.4 mL)-THF (2.0 mL), yielding a flocculant precipitate. Diethyl ether (10 mL) was added, and the reaction mixture was allowed to stir at room temperature for 30 minutes. The precipitate was removed via vacuum filtration, the filtrate was dried over MgSO₄, and concentrated under vacuo to yield 3 which was used without purification. The above obtained 3 was dissolved in 95% EtOH (10 mL), and 10% palladium on activated charcoal (0.110 eq) was added. The reaction flask was evacuated then backfilled with hydrogen. After stirring at room temperature for 2 hours, the catalyst was removed through filtration and solvent removed in vacuo to yield 4, which was purified by liquid chromatography on C18 silica gel using a water-acetonitrile+0.1% formic acid eluent system.

Analysis was carried out using high-resolution LC-HESI-LTQ-Orbitrap-XL MS (Thermo Fisher Scientific), employing a modified version of a method described previously (Chang et al., 2015, Plant Physiol. 169:1127-1140), with the exception that liquid chromatography was carried out using an UltiMate 3000 HPLC (Thermo Fisher Scientific) equipped with a Poroshell 120 SB-C18 column (Agilent Technologies) instead of an Accela HPLC system (Thermo Fisher Scientific) equipped with a Zorbax C18 column (Agilent Technologies). Briefly, 10 microliters of final reaction product was injected at a flow rate of 0.5 mL/min and a gradient of solvent A (water with 0.1% of formic acid) and solvent B (ACN with 0.1% formic Acid) as follows: 100% to 0% (v/v) solvent A over 5 min; isocratic at 0% (v/v) for 1 min; 0% to 100% (v/v) over 0.1 min; and isocratic at 100% (v/v) for 1.9 min. Total run time was 8 minutes. Heated ESI source and interface conditions were operated in positive ion mode as follows: vaporizer temperature, 400° C.; source voltage, 3 kV; sheath gas, 60 au, auxiliary gas, 20 au; capillary temperature, 380° C.; capillary voltage, 6 V; tube lens, 45 V. Instrumentation was performed as a single, HR scan event using Orbitrap detection of m/z in the range of 100-700 m/z. Ion injection time was 300 ms with scan time of 1 s. External and internal calibration procedures ensured <2 ppm error to facilitate elemental formulae predictions. Singly protonated product with exact m/z and expected elemental formula matching protonated 3-(2-[N-ethyl(isopropyl)amino]ethyl)-1H-indol-4-ol (R-group “D” in FIG. 11) having chemical formula (V):

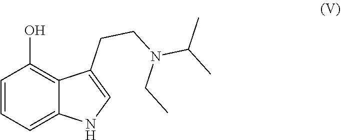 [SMILES: CCN(CCc1c[nH]c2cccc(O)c12)C(C)C]

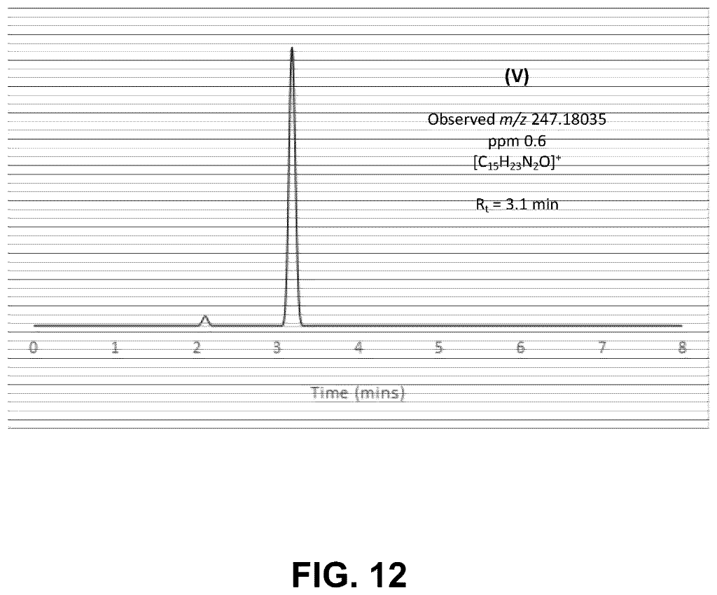

eluted at 3.1 minutes (EIC, see: FIG. 12).

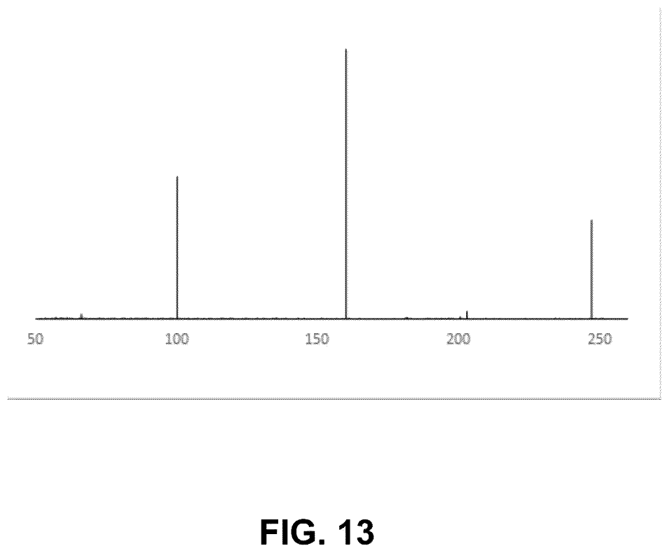

As per standard procedures (Menéndez-Perdomo et al. 2021, Mass Spectrom 56: e4683) high energy collisions (HCD) were achieved in a dedicated, post-LTQ, nitrogen collision cell. Orbitrap-based, HR fragment detection was employed (normalized collision energy, NCE 35), enabling opportunity to assign elemental formulae to subsequent diagnostic ion species characteristic of a compound of formula (V) (FIG. 13, Table I) ((Servillo L. et al., J. Agric. Food Chem 61:5156-5162).

TABLE I
relative abundance of molecular species
in a sample containing compound (V)

| | % Relative | | Empirical |
|---|---|---|---|
| m/z | abundance | Fragment | formula |
| 160.0755 | 100. | [M + H —NH₂C₅H₁₀]⁺ | C₁₀H₁₁NO |
| 100.1117 | 53 | | C₆H₁₄N |
| 247.1805 | 36 | [M + H]⁺ | C₁₅H₂₂N₂O |
| 202.9303 | 2 | | |
| 66.1250 | 1 | | |
| 200.5065 | 0.9 | | |
| 56.9876 | 0.7 | | |

### Example 5—Process for Making a Fifth Hydroxylated Psilocybin Derivative Using Synthetic Chemistry

Chemical synthesis was conducted as described in Example 4, except that cycloheptylamine (9.00 total eq, required for R group “F” in FIG. 11) was used in place of N-ethyl(isopropyl)amine. Analysis was carried out using high-resolution LC-HESI-LTQ-Orbitrap-XL MS (Thermo Fisher Scientific) as described in Example 4. Singly protonated product with exact m/z and expected elemental formula matching protonated 3-[2-(cycloheptylamino)ethyl]-1H-indol-4-ol (R-group “F” in FIG. 11) and having chemical formula (IX):

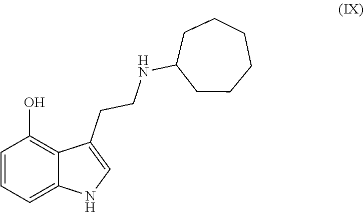 [SMILES: Oc1cccc2[nH]cc(CCNC3CCCCCC3)c12]

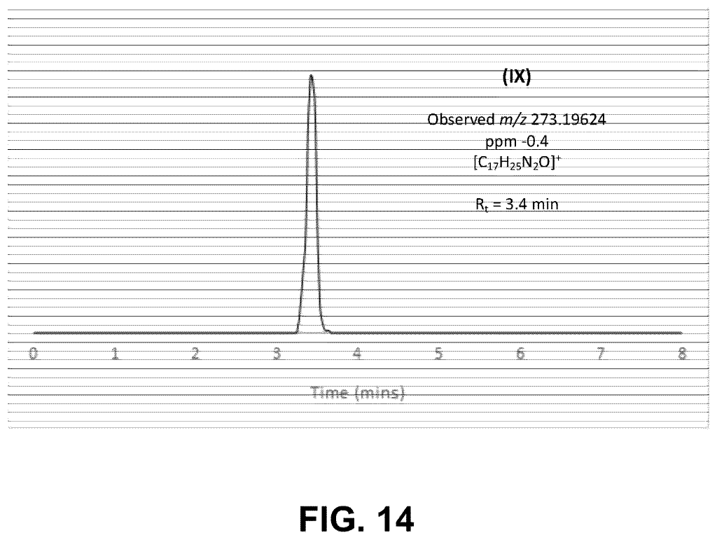

eluted at 3.4 minutes (EIC, see: FIG. 14).

### Example 6—Process for Making a Sixth Hydroxylated Psilocybin Derivative Using Synthetic Chemistry

Chemical synthesis was conducted as described in Example 4, except that dioctylamine (9.00 total eq, required for R group “K” in FIG. 11) was used in place of N-ethyl(isopropyl)amine. Analysis was carried out using high-resolution LC-HESI-LTQ-Orbitrap-XL MS (Thermo Fisher Scientific) as described in Example 4. Singly protonated product with exact m/z and expected elemental formula matching protonated 3-[2-(dioctylamino)ethyl]-1H-indol-4-ol (R-group “K” in FIG. 11) and having chemical formula (X):

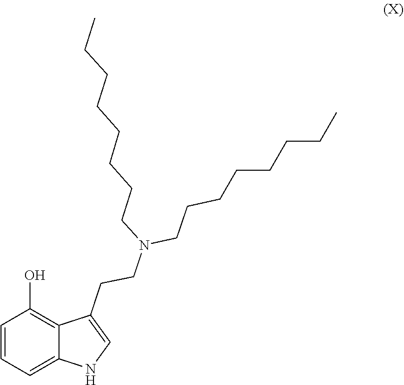 [SMILES: CCCCCCCCN(CCCCCCCC)CCc1c[nH]c2cccc(O)c12]

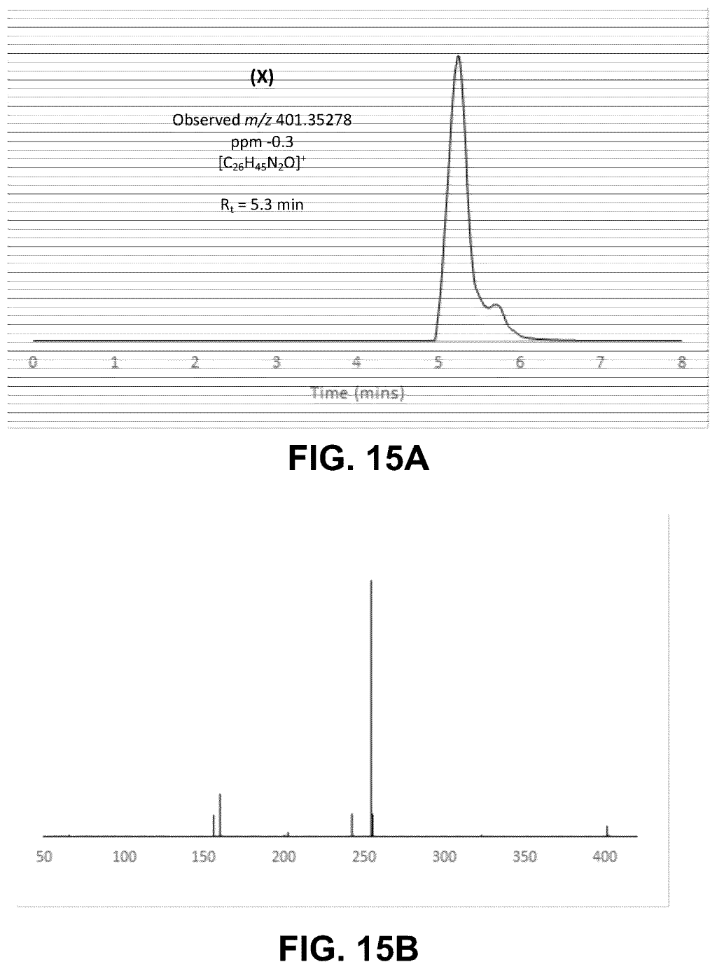

eluted at 5.3 minutes (EIC, see: FIG. 15A).

High energy collisions (HCD) were achieved as described in Example 4. Elemental formulae were assigned to subsequent diagnostic ion species characteristic of a compound of formula (X) (FIG. 15B, Table II).

TABLE II
relative abundance of molecular species
in a sample containing compound (X)

| | % Relative | | Empirical |
|---|---|---|---|
| m/z | abundance | Fragment | formula |
| 254.2842 | 100 | | |
| 160.0755 | 16 | [M + H —NH₂C₁₆H₃₃]⁺ | C₁₀H₁₁NO |
| 242.2842 | 9 | | |
| 255.2875 | 9 | | |
| 156.1745 | 8 | [M + H —C₈H₁₈]⁺ | C₁₆H₂₇N₂O |
| 401.3524 | 4 | [M + H]⁺ | C₂₆H₄₅N₂O |
| 202.4264 | 1.7 | | |
| 66.1240 | 0.6 | | |
| 200.0434 | 0.6 | | |

### Example 7—Process for Making a Seventh Hydroxylated Psilocybin Derivative Using Synthetic Chemistry

Chemical synthesis was conducted as described in Example 4, except that phenethylamine (9.00 total eq, required for R group “H” in FIG. 11) was used in place of N-ethyl(isopropyl)amine. Analysis was carried out using high-resolution LC-HESI-LTQ-Orbitrap-XL MS (Thermo Fisher Scientific) as described in Example 4. Singly protonated product with exact m/z and expected elemental formula matching protonated 3-[2-(phenethylamino)ethyl]-1H-indol-4-ol (R-group “H” in FIG. 11) and having chemical formula (XIII):

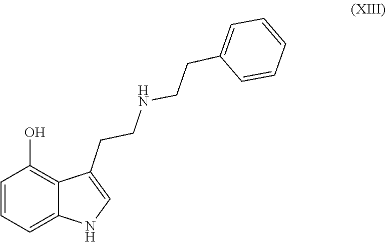 [SMILES: Oc1cccc2[nH]cc(CCNCCc3ccccc3)c12]

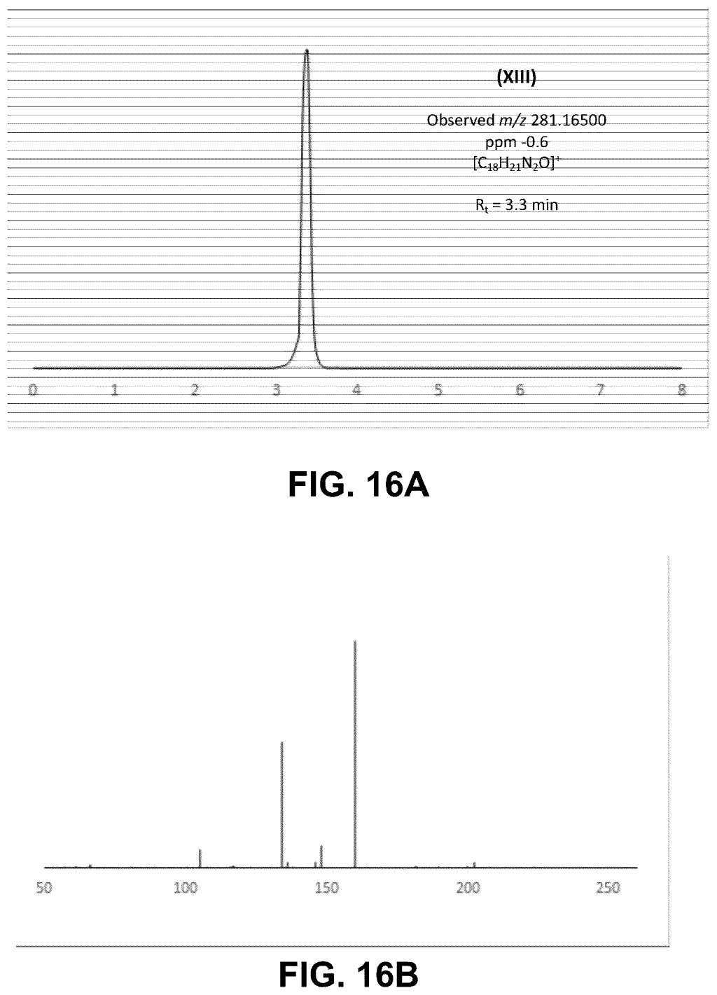

eluted at 3.3 minutes (EIC, see: FIG. 16A).

High energy collisions (HCD) were achieved as described in Example 4. Elemental formulae were assigned to subsequent diagnostic ion species characteristic of a compound of formula (XIII) (FIG. 16B, Table III).

TABLE III
relative abundance of molecular species
in a sample containing compound (XIII)

| | % Relative | | Empirical |
|---|---|---|---|
| m/z | abundance | Fragment | formula |
| 160.0755 | 100 | [M + H —NH₂C₂H₄Ben]⁺ | C₁₀H₁₁NO |
| 134.0962 | 6 | | C₉H₁₂N |
| 148.0755 | 10 | [M + H —CH₂NH₂C₃H₆Ben]⁺ | C₉H₉NO |
| 281.1649 | 9 | [M + H]⁺ | C₁₈H₂₀N₂O |
| 105.0696 | 8 | | C₈H₉ |
| 146.0598 | 3 | | |
| 136.1118 | 2 | | |
| 202.4219 | 2 | | |

### Example 8—Process for Making an Eighth Hydroxylated Psilocybin Derivative Using Synthetic Chemistry

Chemical synthesis was conducted as described in Example 4, except that 2-phenylpropylamine (9.00 total eq, required for R group “I” in FIG. 11) was used in place of N-ethyl(isopropyl)amine. Analysis was carried out using high-resolution LC-HESI-LTQ-Orbitrap-XL MS (Thermo Fisher Scientific) as described in Example 4. Singly protonated product with exact m/z and expected elemental formula matching protonated 3-[2-(2-phenylpropylamino)ethyl]-1H-indol-4-ol (R-group “I” in FIG. 11) and having chemical formula (XIV):

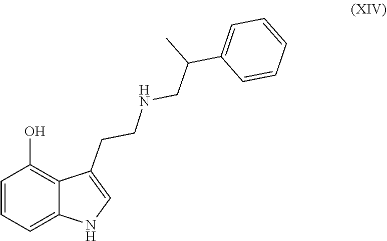 [SMILES: CC(CNCCc1c[nH]c2cccc(O)c12)c1ccccc1]

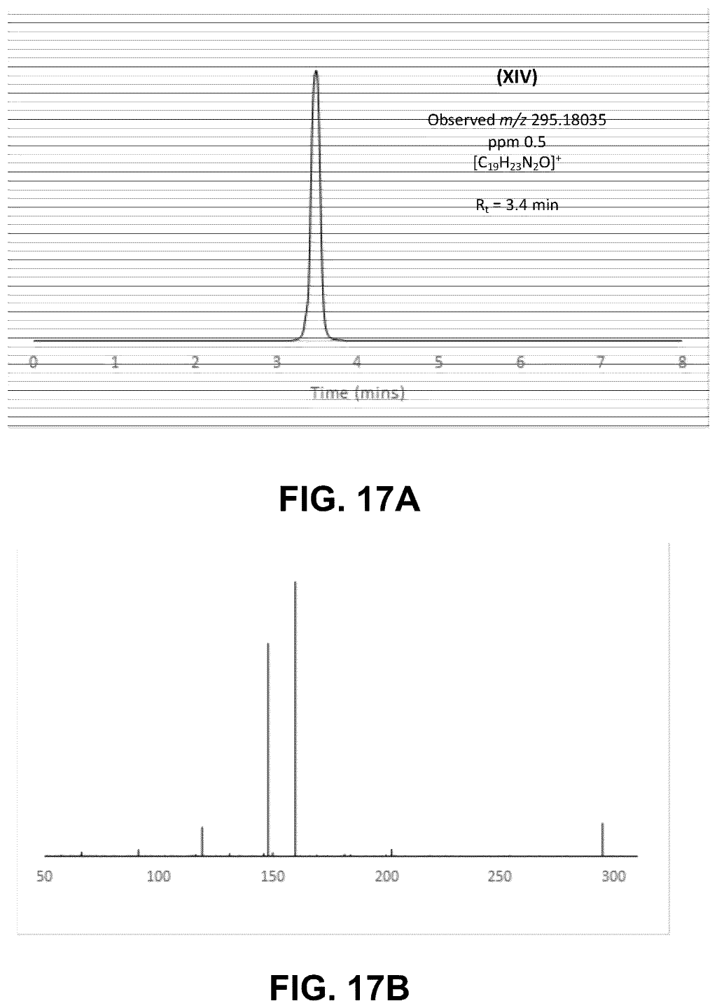

eluted at 3.4 minutes (EIC, see: FIG. 17A).

High energy collisions (HCD) were achieved as described in Example 4. Elemental formulae were assigned to subsequent diagnostic ion species characteristic of a compound of formula (XIV) (FIG. 17B; Table IV).

TABLE IV
relative abundance of molecular species
in a sample containing compound (XIV)

| | % Relative | | Empirical |
|---|---|---|---|
| m/z | abundance | Fragment | formula |
| 160.0755 | 100 | [M + H —OH —C₃H₆Ben]⁺ | C₁₀H₁₁N₂ |
| 148.1119 | 77 | [M + H —CH₂NH₂C₃H₆Ben]⁺ | C₉H₉NO |
| 295.1806 | 12 | [M + H]⁺ | C₁₉H₂₃N₂O |
| 119.0852 | 11 | | C₉H₁₁ |
| 148.0755 | 4 | | |
| 202.4136 | 3 | | |
| 91.0539 | 2 | | C₇H₇ |
| 160.0736 | 2 | | |
| 148.1102 | 2 | | |

### Example 9—Process for Making a Ninth Hydroxylated Psilocybin Derivative Using Synthetic Chemistry

Chemical synthesis was conducted as described in Example 4, except that N-propylbutylamine (9.00 total eq, required for R group “G” in FIG. 11) was used in place of N-ethyl(isopropyl)amine. Analysis was carried out using high-resolution LC-HESI-LTQ-Orbitrap-XL MS (Thermo Fisher Scientific) as described in Example 4. Singly protonated product with exact m/z and expected elemental formula matching protonated 3-[2-(N-butyl-N-propylamino)ethyl]-1H-indol-4-ol (R-group “G” in FIG. 11) and having chemical formula (XV):

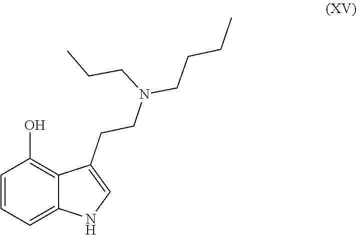 [SMILES: CCCCN(CCC)CCc1c[nH]c2cccc(O)c12]

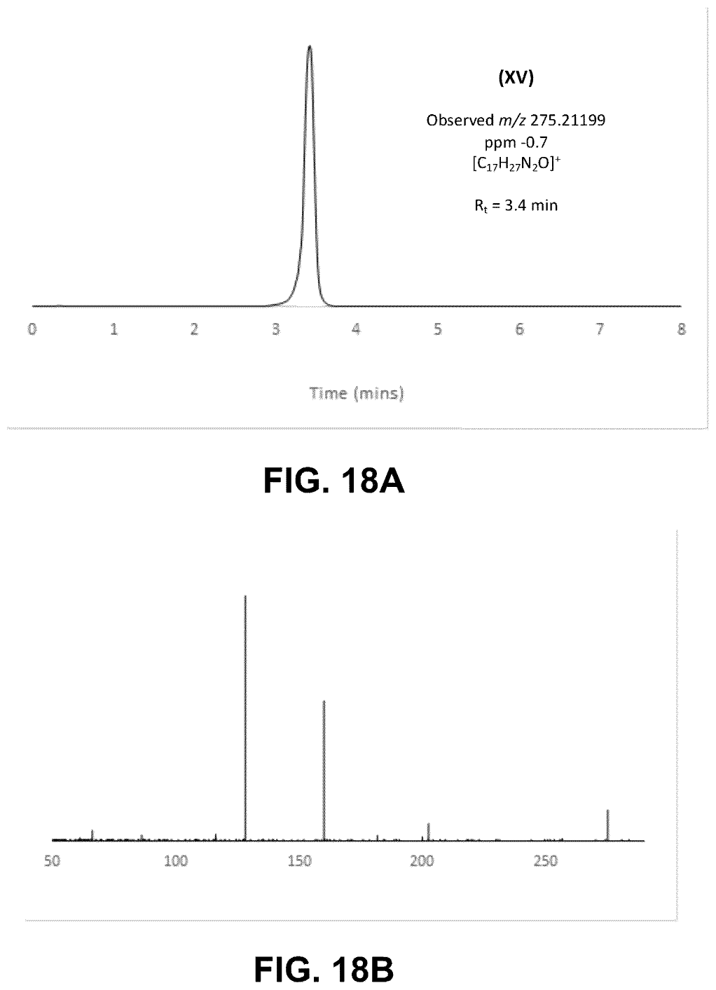

eluted at 3.4 minutes (EIC, see: FIG. 18A).

High energy collisions (HCD) were achieved as described in Example 4. Elemental formulae were assigned to subsequent diagnostic ion species characteristic of a compound of formula (XV) (FIG. 18B, Table V).

TABLE V
relative abundance of molecular species
in a sample containing compound (XV)

| | % | | |
|---|---|---|---|
| | Relative | | Empirical |
| m/z | abundance | Fragment | formula |
| 128.1431 | 100 | [M + H —OH —CH₂NH₂C₇H₁₆]⁺ | C₈H₁₈N |
| 160.0755 | 58 | [M + H —NH₂C₇H₁₆]⁺ | C₁₀H₁₁NO |
| 275.2119 | 13 | [M + H]⁺ | C₁₇H₂₇N₂O |
| 202.4525 | 7 | | |
| 66.1247 | 4 | | |
| 116.1430 | 3 | | C₇H₁₈N |
| 86.0960 | 3 | | C₅H₁₂N |
| 181.7339 | 2 | | |
| 200.0026 | 2 | | |

### Example 10—Process for Making a Tenth Hydroxylated Psilocybin Derivative Using Synthetic Chemistry

Chemical synthesis was conducted as described in Example 4, except that isopentylamine (9.00 total eq, required for R group “E” in FIG. 11) was used in place of N-ethyl(isopropyl)amine. Analysis was carried out using high-resolution LC-HESI-LTQ-Orbitrap-XL MS (Thermo Fisher Scientific) as described in Example 4. Singly protonated product with exact m/z and expected elemental formula matching protonated 3-[2-(isopentylamino)ethyl]-1H-indol-4-ol (R-group “E” in FIG. 11) and having chemical formula (XVI):

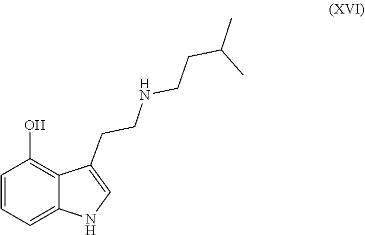 [SMILES: CC(C)CCNCCc1c[nH]c2cccc(O)c12]

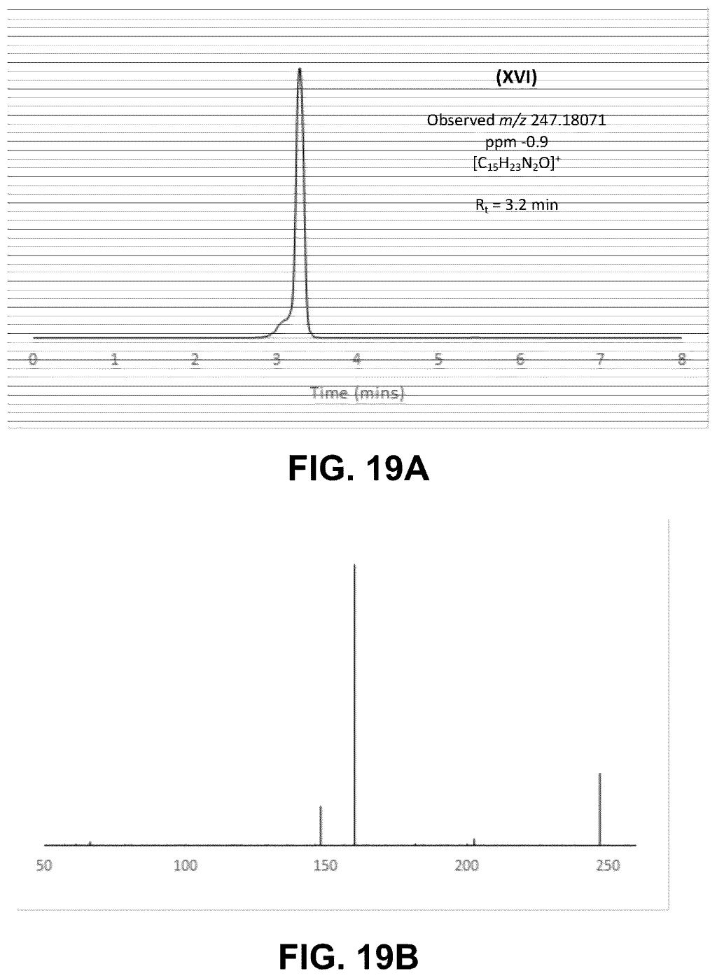

eluted at 3.2 minutes (EIC, see: FIG. 19A).

High energy collisions (HCD) were achieved as described in Example 4. Elemental formulae were assigned to subsequent diagnostic ion species characteristic of a compound of formula (XVI), (FIG. 19B, Table VI).

TABLE VI
relative abundance of molecular species
in a sample containing compound (XVI)

| | % Relative | | Empirical |
|---|---|---|---|
| m/z | abundance | Fragment | formula |
| 160.0755 | 100 | [M + H —NH₂C₅H₁₁]⁺ | C₁₀H₁₁NO |
| 247.1805 | 26 | [M + H]⁺ | C₁₅H₂₂N₂O |
| 148.0755 | 14 | [M + H —CH₂NH₂C₅H₁₁]⁺ | C₉H₉NO |
| 202.5658 | 2 | | |
| 160.0736 | 2 | | |
| 66.1248 | 1 | | |
| 160.0727 | 0.7 | | |
| 200.1163 | 0.6 | | |
| 181.7336 | 0.6 | | |

### Example 11—Process for Making an Eleventh Hydroxylated Psilocybin Derivative Using Synthetic Chemistry

Chemical synthesis was conducted as described in Example 4, except that cyclopentylamine (9.00 total eq, required for R group “C” in FIG. 11) was used in place of N-ethyl(isopropyl)amine. Analysis was carried out using high-resolution LC-HESI-LTQ-Orbitrap-XL MS (Thermo Fisher Scientific) as described in Example 4. Singly protonated product with exact m/z and expected elemental formula matching protonated 3-[2-(cyclopentylamino)ethyl]-1H-indol-4-ol (R-group “C” in FIG. 11) and having chemical formula (XVIII):

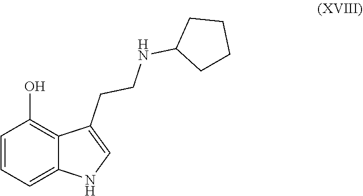 [SMILES: Oc1cccc2[nH]cc(CCNC3CCCC3)c12]

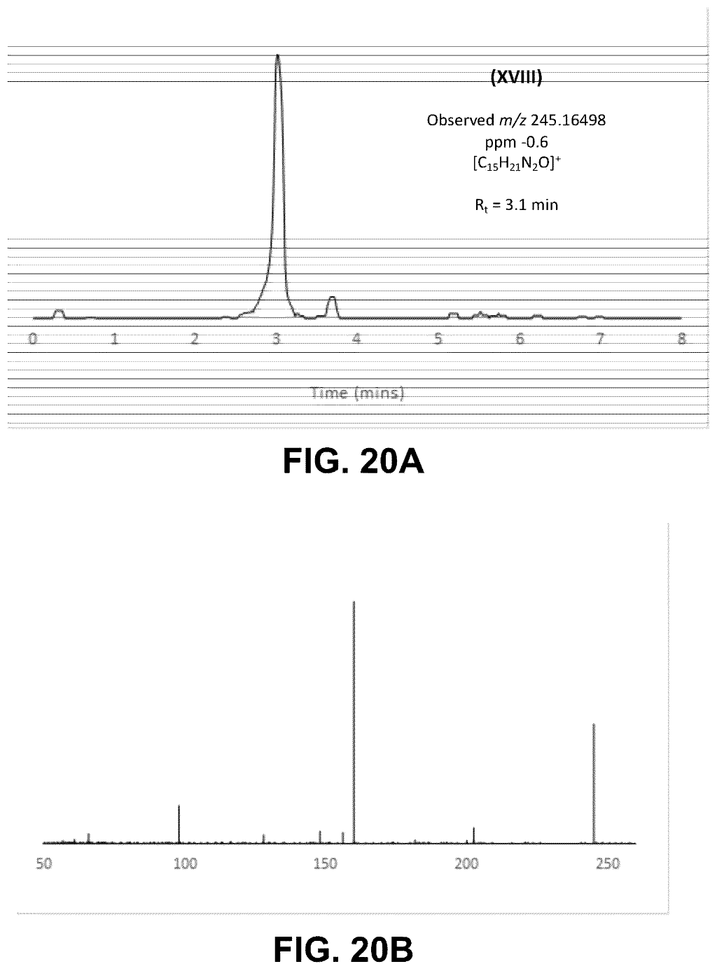

eluted at 3.1 minutes (EIC, see: FIG. 20A).

High energy collisions (HCD) were achieved as described in Example 4. Elemental formulae were assigned to subsequent diagnostic ion species characteristic of a compound of formula (XVIII) (FIG. 20B, Table VII).

TABLE VII
relative abundance of molecular species
in a sample containing compound (XVIII)

| | % Relative | | Empirical |
|---|---|---|---|
| m/z | abundance | Fragment | formula |
| 160.0755 | 100 | [M + H —NH₂C₅H₉]⁺ | C₁₀H₁₁NO |
| 245.1658 | 49 | [M + H]⁺ | C₁₅H₂₁N₂O |
| 98.0960 | 16 | | C₆H₁₂N |
| 202.5918 | 7 | | |
| 148.0754 | 5 | | C₉H₁₀ON |
| 156.1745 | 5 | | |
| 66.1249 | 4 | | |
| 128.1432 | 4 | | C₈H₁₈N |

### Example 12—Process for Making a Twelfth Hydroxylated Psilocybin Derivative Using Synthetic Chemistry

Chemical synthesis was conducted as described in Example 4, except that butylamine (9.00 total eq, required for R group “A” in FIG. 11) was used in place of N-ethyl(isopropyl)amine. Analysis was carried out using high-resolution LC-HESI-LTQ-Orbitrap-XL MS (Thermo Fisher Scientific) as described in Example 4. Singly protonated product with exact m/z and expected elemental formula matching protonated 3-[2-(butylamino)ethyl]-1H-indol-4-ol (R-group “A” in FIG. 11) and having chemical formula (XXVI):

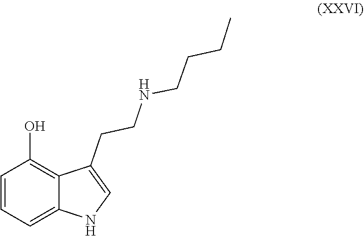 [SMILES: CCCCNCCc1c[nH]c2cccc(O)c12]

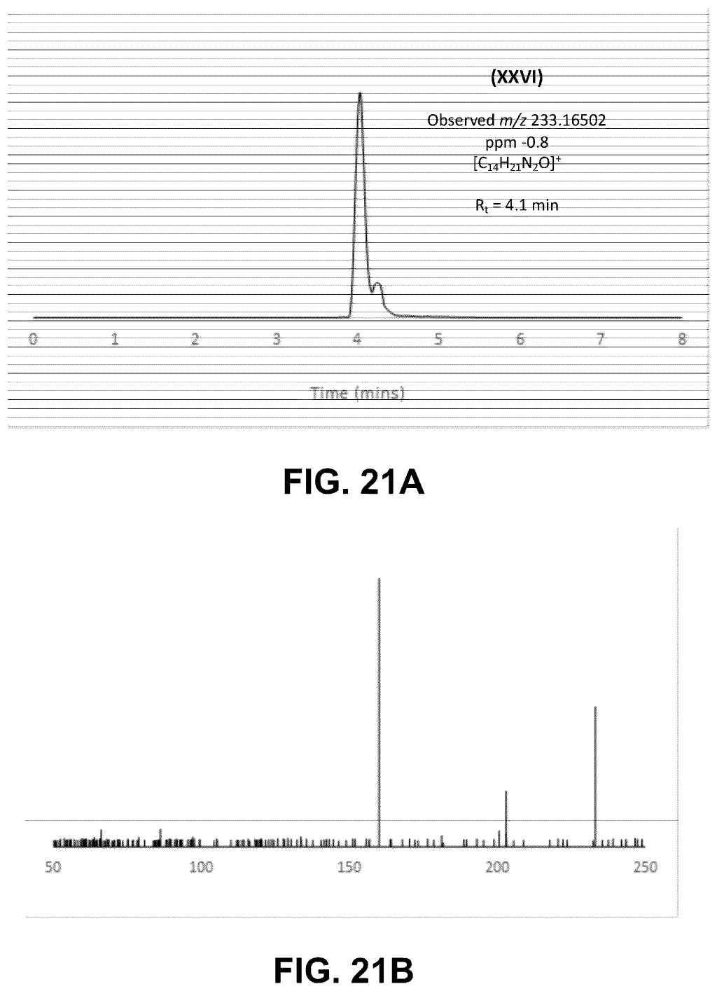

eluted at 4.1 minutes (EIC, see: FIG. 21A).

High energy collisions (HCD) were achieved as described in Example 4. Elemental formulae were assigned to subsequent diagnostic ion species characteristic of a compound of formula (XXVI) (FIG. 21B, Table VIII).

TABLE VIII
relative abundance of molecular species
in a sample containing compound (XXVI)

| | % Relative | | Empirical |
|---|---|---|---|
| m/z | abundance | Fragment | formula |
| 160.0755 | 100 | [M + H —NH₂C₄H₉]⁺ | C₁₀H₁₁NO |
| 233.1649 | 51 | [M + H]⁺ | C₁₄H₂₁N₂O |
| 203.0414 | 21 | [M + H —C₂H₆]⁺ | C₁₂H₁₅N₂O |
| 86.0962 | 6 | | C₅H₁₂N |
| 66.1252 | 6 | | |
| 200.6570 | 6 | | |
| 202.9407 | 5 | | |

### Example 13—Process for Making a Thirteenth Hydroxylated Psilocybin Derivative Using Synthetic Chemistry

Chemical synthesis was conducted as described in Example 4, except that dodecylamine (9.00 total eq, required for R group “L” in FIG. 11) was used in place of N-ethyl(isopropyl)amine. Analysis was carried out using high-resolution LC-HESI-LTQ-Orbitrap-XL MS (Thermo Fisher Scientific) as described in Example 4. Singly protonated product with exact m/z and expected elemental formula matching protonated 3-[2-(dodecylamino)ethyl]-1H-indol-4-ol (R-group “L” in FIG. 11) and having chemical formula (XXVII):

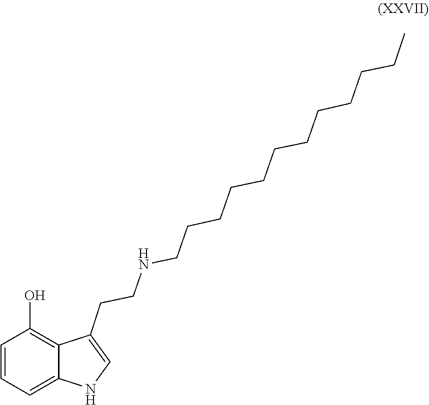 [SMILES: CCCCCCCCCCCCNCCc1c[nH]c2cccc(O)c12]

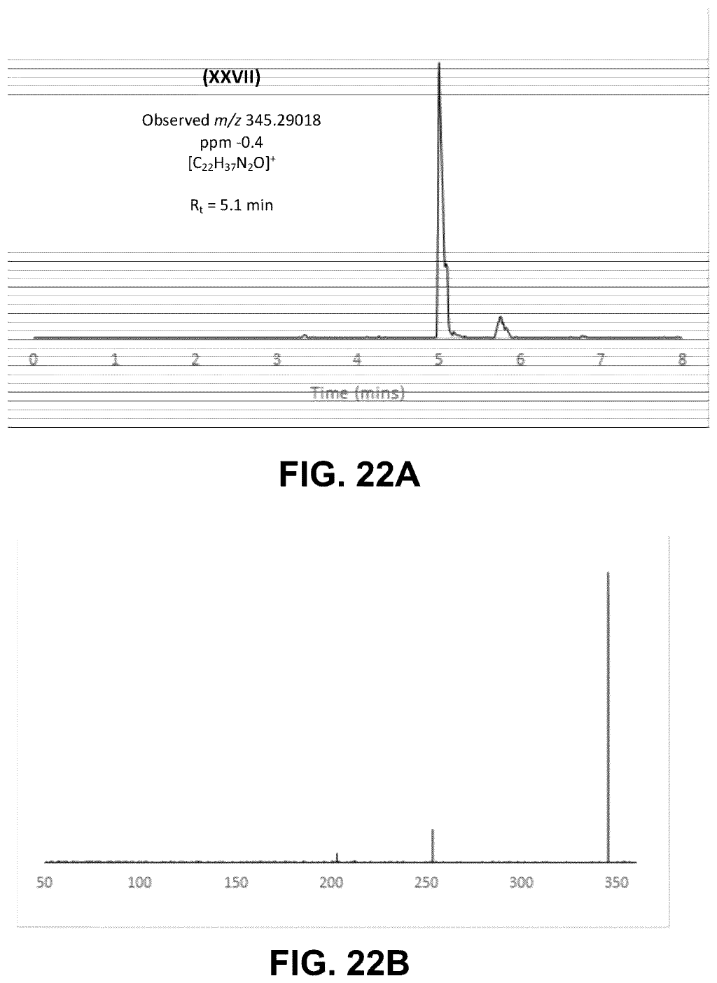

eluted at 5.1 minutes (EIC, see: FIG. 22A).

High energy collisions (HCD) were achieved as described in Example 4. Elemental formulae were assigned to subsequent diagnostic ion species characteristic of a compound of formula (XXVII), (FIG. 22B, Table IX).

TABLE IX
relative abundance of molecular species
in a sample containing compound (XXVII)

| | % Relative | | Empirical |
|---|---|---|---|
| m/z | abundance | Fragment | formula |
| 345.3265 | 100 | [M + H]⁺ | C₂₂H₃₆N₂O |
| 253.2640 | 12 | | C₁₆H₃₃N₂ |
| 203.0388 | 3 | | |
| 345.2809 | 1 | | |
| 200.6552 | 0.8 | | |
| 345.3366 | 0.7 | | |
| 56.9881 | 0.6 | | |

### Example 14-Process for Making a Fourteenth Hydroxylated Psilocybin Derivative Using Synthetic Chemistry

Chemical synthesis was conducted as described in Example 4, except that dihexylamine (9.00 total eq, required for R group “J” in FIG. 11) was used in place of N-ethyl(isopropyl)amine. Analysis was carried out using high-resolution LC-HESI-LTQ-Orbitrap-XL MS (Thermo Fisher Scientific) as described in Example 4. Singly protonated product with exact m/z and expected elemental formula matching protonated 3-[2-(dihexylamino)ethyl]-1H-indol-4-ol (R-group “J” in FIG. 11) and having chemical formula (XXIX):

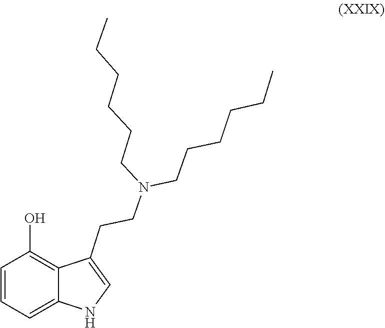 [SMILES: CCCCCCN(CCCCCC)CCc1c[nH]c2cccc(O)c12]

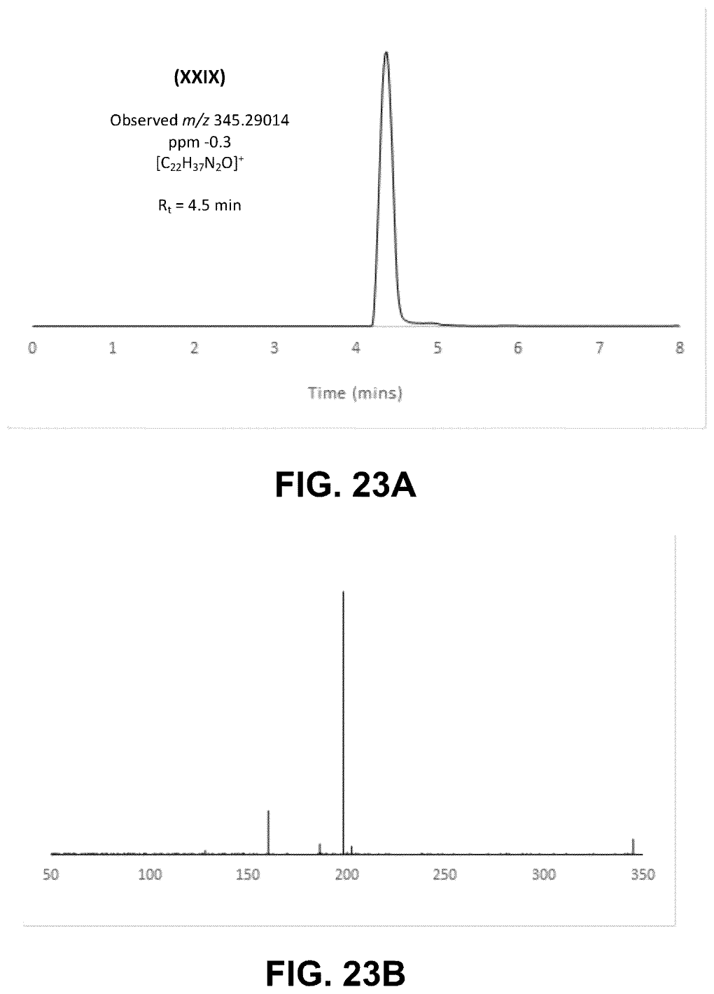

eluted at 4.5 minutes (EIC, see: FIG. 23A).

High energy collisions (HCD) were achieved as described in Example 4. Elemental formulae were assigned to subsequent diagnostic ion species characteristic of a compound of formula (XXIX), (FIG. 23B, Table X).

TABLE X
relative abundance of molecular species
in a sample containing compound (XXIX)

| | % Relative | | Empirical |
|---|---|---|---|
| m/z | abundance | Fragment | formula |
| 198.2215 | 100 | | |
| 160.0755 | 17 | [M + H —NH₂C₁₂H₂₆]⁺ | C₁₀H₁₁NO |
| 345.2904 | 6 | [M + H]⁺ | C₂₂H₃₇N₂O |
| 186.2215 | 4 | | |
| 202.4168 | 3 | | |
| 128.1431 | 2 | | |
| 200.0211 | 1 | | |
| 202.5173 | 0.8 | | |
| 198.2173 | 0.8 | | |

1. A chemical compound or salt thereof having formula (I): 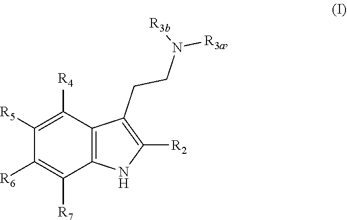
wherein one of R₄ and R₅ is a hydroxy group, wherein R₄ or R₅ when they are not a hydroxy group are a hydrogen atom or an alkyl group, R₂, R₆, and R₇ are a hydrogen atom or an alkyl group, and wherein R₃ₐ and R_{3b} are each independently an alkyl group, a cycloalkyl group, an alkaryl group, an aryl group, an acyl group, or a hydrogen atom.

2. A chemical compound according to claim 1, wherein R₄ is a hydroxy group.

3. A chemical compound according to claim 2, wherein R₃ₐ is an acyl group having the formula —C(═O)CₙC₂ₙ₊₁, wherein n is an integer from 1 to 6.

4. A chemical compound according to claim 2, wherein R₅ a is an acyl group having the formula —C(═O)CₙC₂ₙ₊₁, wherein n is an integer from 1 to 3.

5. A chemical compound according to claim 2, wherein R₅ a is an acetyl group (—C(═O)CH₃).

6. A chemical compound according to claim 2, wherein R₅ a is an acyl group having the formula —C(═O)CₙC₂ₙ₊₁, wherein n is an integer from 1 to 6, and wherein R_{3b} is a hydrogen atom.

7. A chemical compound according to claim 2, wherein R₅ a is an acyl group having the formula —C(═O)CₙC₂ₙ₊₁, wherein n is an integer from 1 to 3, and wherein R_{3b} is a hydrogen atom.

8. A chemical compound according to claim 2, wherein R₅ a is an acetyl group (—C(═O)CH₃), and wherein R_{3b} is a hydrogen atom.

9. A chemical compound according to claim 2, wherein one of R₂, R₅, R₆, or R₇ is an alkyl group, and the remaining R₂, R₅, R₆, and R₇ are a hydrogen atom.

10. A chemical compound according to claim 9, wherein the alkyl group is a (C₁-C₆)-alkyl group.

11. A chemical compound according to claim 9, wherein the alkyl group is a (C₁-C₃)-alkyl group.

12. A chemical compound according to claim 9, wherein the alkyl group is a methyl group (CH₃).

13. A chemical compound according to claim 2, wherein R₇ is an alkyl group, and R₂, R₅, and R₆, are a hydrogen atom.

14. A chemical compound according to claim 2, wherein R₇ is a (C₁-C₆)-alkyl group, and R₂, R₅, R₆, are a hydrogen atom.

15. A chemical compound according to claim 2, wherein R₇ is a (C₁-C₃)-alkyl group, and R₂, R₅, and R₆, are a hydrogen atom.

16. A chemical compound according to claim 2, wherein R₇ is a methyl group (CH₃), and R₂, R₅, and R₆, are a hydrogen atom.

17. A chemical compound according to claim 2, wherein R₂, R₅, R₆, and R₇ each are a hydrogen atom, and wherein, optionally, R₃ₐ is an acyl group having the formula —C(═O)CₙC₂ₙ₊₁, wherein n is an integer from 1 to 6, and wherein R_{3b} is a hydrogen atom.

18. A chemical compound according to claim 1, wherein the chemical compound is selected from the group consisting of compounds having formulas (IV) and (XXXIV):

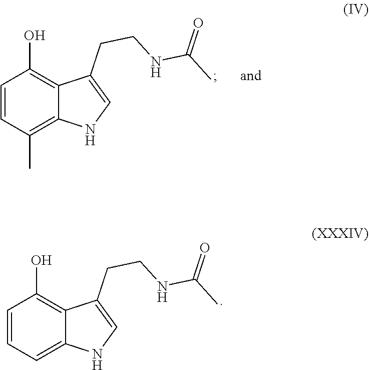 [SMILES: CC(=O)NCCc1c[nH]c2c(C)ccc(O)c12 ; CC(=O)NCCc1c[nH]c2cccc(O)c12]

19. A pharmaceutical drug formulation comprising an effective amount of a chemical compound according to claim 1, together with a pharmaceutically acceptable diluent, carrier, or excipient.

20. A method for treating a psychiatric disorder, the method comprising administering to a subject in need thereof a pharmaceutical formulation comprising a chemical compound according to claim 1, wherein the pharmaceutical formulation is administered in an effective amount to treat the psychiatric disorder in the subject.
